SEQUENCE LISTING (287 sequences)

SEQ ID NO: 1 - protein, 8 aa, Artificial Sequence
  Description of Artificial Sequence: Synthetic peptide
  MOD_RES (3)..(3): Asp or Glu
  MOD_RES (6)..(6): Asp or Glu
NGXPRXPR

SEQ ID NO: 2 - protein, 8 aa, Artificial Sequence
  Description of Artificial Sequence: Synthetic peptide
NGDPRDPR

SEQ ID NO: 3 - protein, 8 aa, Artificial Sequence
  Description of Artificial Sequence: Synthetic peptide
NGDPREPR

SEQ ID NO: 4 - protein, 8 aa, Artificial Sequence
  Description of Artificial Sequence: Synthetic peptide
NGEPRDPR

SEQ ID NO: 5 - protein, 8 aa, Artificial Sequence
  Description of Artificial Sequence: Synthetic peptide
NGEPREPR

SEQ ID NO: 6 - protein, 23 aa, Artificial Sequence
  Description of Artificial Sequence: Synthetic peptide
PINGDPRDPRRGQNRSARIAKQL

SEQ ID NO: 7 - protein, 23 aa, Artificial Sequence
  Description of Artificial Sequence: Synthetic peptide
PINGEPRDPRRGQNRSARIAKQL

SEQ ID NO: 8 - protein, 23 aa, Artificial Sequence
  Description of Artificial Sequence: Synthetic peptide
PINGDPREPRRGQNRSARIAKQL

SEQ ID NO: 9 - protein, 23 aa, Artificial Sequence
  Description of Artificial Sequence: Synthetic peptide
PINGEPREPRRGQNRSARIAKQL

SEQ ID NO: 10 - protein, 23 aa, Artificial Sequence
  Description of Artificial Sequence: Synthetic peptide
PFNGDPRDPRRGQNRSARIAKQL

SEQ ID NO: 11 - protein, 23 aa, Artificial Sequence
  Description of Artificial Sequence: Synthetic peptide
PFNGEPRDPRRGQNRSARIAKQL

SEQ ID NO: 12 - protein, 23 aa, Artificial Sequence
  Description of Artificial Sequence: Synthetic peptide
PFNGDPREPRRGQNRSARIAKQL

SEQ ID NO: 13 - protein, 23 aa, Artificial Sequence
  Description of Artificial Sequence: Synthetic peptide
PFNGEPREPRRGQNRSARIAKQL

SEQ ID NO: 14 - protein, 8 aa, Artificial Sequence
  Description of Artificial Sequence: Synthetic peptide
NGSPRDPR

SEQ ID NO: 15 - protein, 8 aa, Artificial Sequence
  Description of Artificial Sequence: Synthetic peptide
NGSPREPR

SEQ ID NO: 16 - protein, 8 aa, Artificial Sequence
  Description of Artificial Sequence: Synthetic peptide
NGDPRTPR

SEQ ID NO: 17 - protein, 8 aa, Artificial Sequence
  Description of Artificial Sequence: Synthetic peptide
NGEPRTPR

SEQ ID NO: 18 - protein, 23 aa, Artificial Sequence
  Description of Artificial Sequence: Synthetic peptide
PINGSPRDPRRGQNRSARIAKQL

SEQ ID NO: 19 - protein, 23 aa, Artificial Sequence
  Description of Artificial Sequence: Synthetic peptide
PINGSPREPRRGQNRSARIAKQL

SEQ ID NO: 20 - protein, 23 aa, Artificial Sequence
  Description of Artificial Sequence: Synthetic peptide
PINGDPRTPRRGQNRSARIAKQL

SEQ ID NO: 21 - protein, 23 aa, Artificial Sequence
  Description of Artificial Sequence: Synthetic peptide
PINGEPRTPRRGQNRSARIAKQL

SEQ ID NO: 22 - protein, 23 aa, Artificial Sequence
  Description of Artificial Sequence: Synthetic peptide
PFNGSPRDPRRGQNRSARIAKQL

SEQ ID NO: 23 - protein, 23 aa, Artificial Sequence
  Description of Artificial Sequence: Synthetic peptide
PFNGSPREPRRGQNRSARIAKQL

SEQ ID NO: 24 - protein, 23 aa, Artificial Sequence
  Description of Artificial Sequence: Synthetic peptide
PFNGDPRTPRRGQNRSARIAKQL

SEQ ID NO: 25 - protein, 23 aa, Artificial Sequence
  Description of Artificial Sequence: Synthetic peptide
PFNGEPRTPRRGQNRSARIAKQL

SEQ ID NO: 26 - protein, 22 aa, Artificial Sequence
  Description of Artificial Sequence: Synthetic peptide
INGDPRDPRRGQNRSARIAKQL

SEQ ID NO: 27 - protein, 22 aa, Artificial Sequence
  Description of Artificial Sequence: Synthetic peptide
INGEPRDPRRGQNRSARIAKQL

SEQ ID NO: 28 - protein, 22 aa, Artificial Sequence
  Description of Artificial Sequence: Synthetic peptide
INGDPREPRRGQNRSARIAKQL

SEQ ID NO: 29 - protein, 22 aa, Artificial Sequence
  Description of Artificial Sequence: Synthetic peptide
INGEPREPRRGQNRSARIAKQL

SEQ ID NO: 30 - protein, 22 aa, Artificial Sequence
  Description of Artificial Sequence: Synthetic peptide
FNGDPRDPRRGQNRSARIAKQL

SEQ ID NO: 31 - protein, 22 aa, Artificial Sequence
  Description of Artificial Sequence: Synthetic peptide
FNGEPRDPRRGQNRSARIAKQL

SEQ ID NO: 32 - protein, 22 aa, Artificial Sequence
  Description of Artificial Sequence: Synthetic peptide
FNGDPREPRRGQNRSARIAKQL

SEQ ID NO: 33 - protein, 22 aa, Artificial Sequence
  Description of Artificial Sequence: Synthetic peptide
FNGEPREPRRGQNRSARIAKQL

SEQ ID NO: 34 - protein, 22 aa, Artificial Sequence
  Description of Artificial Sequence: Synthetic peptide
INGSPRDPRRGQNRSARIAKQL

SEQ ID NO: 35 - protein, 22 aa, Artificial Sequence
  Description of Artificial Sequence: Synthetic peptide
INGSPREPRRGQNRSARIAKQL

SEQ ID NO: 36 - protein, 22 aa, Artificial Sequence
  Description of Artificial Sequence: Synthetic peptide
INGDPRTPRRGQNRSARIAKQL

SEQ ID NO: 37 - protein, 22 aa, Artificial Sequence
  Description of Artificial Sequence: Synthetic peptide
INGEPRTPRRGQNRSARIAKQL

SEQ ID NO: 38 - protein, 22 aa, Artificial Sequence
  Description of Artificial Sequence: Synthetic peptide
FNGSPRDPRRGQNRSARIAKQL

SEQ ID NO: 39 - protein, 22 aa, Artificial Sequence
  Description of Artificial Sequence: Synthetic peptide
FNGSPREPRRGQNRSARIAKQL

SEQ ID NO: 40 - protein, 22 aa, Artificial Sequence
  Description of Artificial Sequence: Synthetic peptide
FNGDPRTPRRGQNRSARIAKQL

SEQ ID NO: 41 - protein, 22 aa, Artificial Sequence
  Description of Artificial Sequence: Synthetic peptide
FNGEPRTPRRGQNRSARIAKQL

SEQ ID NO: 42 - protein, 21 aa, Artificial Sequence
  Description of Artificial Sequence: Synthetic peptide
NGDPRDPRRGQNRSARIAKQL

SEQ ID NO: 43 - protein, 21 aa, Artificial Sequence
  Description of Artificial Sequence: Synthetic peptide
NGEPRDPRRGQNRSARIAKQL

SEQ ID NO: 44 - protein, 21 aa, Artificial Sequence
  Description of Artificial Sequence: Synthetic peptide
NGDPREPRRGQNRSARIAKQL

SEQ ID NO: 45 - protein, 21 aa, Artificial Sequence
  Description of Artificial Sequence: Synthetic peptide
NGEPREPRRGQNRSARIAKQL

SEQ ID NO: 46 - protein, 21 aa, Artificial Sequence
  Description of Artificial Sequence: Synthetic peptide
NGSPRDPRRGQNRSARIAKQL

SEQ ID NO: 47 - protein, 21 aa, Artificial Sequence
  Description of Artificial Sequence: Synthetic peptide
NGSPREPRRGQNRSARIAKQL

SEQ ID NO: 48 - protein, 21 aa, Artificial Sequence
  Description of Artificial Sequence: Synthetic peptide
NGDPRTPRRGQNRSARIAKQL

SEQ ID NO: 49 - protein, 21 aa, Artificial Sequence
  Description of Artificial Sequence: Synthetic peptide
NGEPRTPRRGQNRSARIAKQL

SEQ ID NO: 50 - protein, 21 aa, Artificial Sequence
  Description of Artificial Sequence: Synthetic peptide
NGDPRDPRRGQNRSARIAKQL

SEQ ID NO: 51 - protein, 21 aa, Artificial Sequence
  Description of Artificial Sequence: Synthetic peptide
NGEPRDPRRGQNRSARIAKQL

SEQ ID NO: 52 - protein, 21 aa, Artificial Sequence
  Description of Artificial Sequence: Synthetic peptide
NGDPREPRRGQNRSARIAKQL

SEQ ID NO: 53 - protein, 21 aa, Artificial Sequence
  Description of Artificial Sequence: Synthetic peptide
NGEPREPRRGQNRSARIAKQL

SEQ ID NO: 54 - protein, 21 aa, Artificial Sequence
  Description of Artificial Sequence: Synthetic peptide
NGSPRDPRRGQNRSARIAKQL

SEQ ID NO: 55 - protein, 21 aa, Artificial Sequence
  Description of Artificial Sequence: Synthetic peptide
NGSPREPRRGQNRSARIAKQL

SEQ ID NO: 56 - protein, 21 aa, Artificial Sequence
  Description of Artificial Sequence: Synthetic peptide
NGDPRTPRRGQNRSARIAKQL

SEQ ID NO: 57 - protein, 21 aa, Artificial Sequence
  Description of Artificial Sequence: Synthetic peptide
NGEPRTPRRGQNRSARIAKQL

SEQ ID NO: 58 - protein, 10 aa, Artificial Sequence
  Description of Artificial Sequence: Synthetic peptide
NGDPRDPRRG

SEQ ID NO: 59 - protein, 10 aa, Artificial Sequence
  Description of Artificial Sequence: Synthetic peptide
NGEPRDPRRG

SEQ ID NO: 60 - protein, 10 aa, Artificial Sequence
  Description of Artificial Sequence: Synthetic peptide
NGDPREPRRG

SEQ ID NO: 61 - protein, 10 aa, Artificial Sequence
  Description of Artificial Sequence: Synthetic peptide
NGEPREPRRG

SEQ ID NO: 62 - protein, 10 aa, Artificial Sequence
  Description of Artificial Sequence: Synthetic peptide
NGSPRDPRRG

SEQ ID NO: 63 - protein, 10 aa, Artificial Sequence
  Description of Artificial Sequence: Synthetic peptide
NGSPREPRRG

SEQ ID NO: 64 - protein, 10 aa, Artificial Sequence
  Description of Artificial Sequence: Synthetic peptide
NGDPRTPRRG

SEQ ID NO: 65 - protein, 10 aa, Artificial Sequence
  Description of Artificial Sequence: Synthetic peptide
NGEPRTPRRG

SEQ ID NO: 66 - protein, 14 aa, Artificial Sequence
  Description of Artificial Sequence: Synthetic peptide
NGDPRDPRRGQNRS

SEQ ID NO: 67 - protein, 14 aa, Artificial Sequence
  Description of Artificial Sequence: Synthetic peptide
NGEPRDPRRGQNRS

SEQ ID NO: 68 - protein, 14 aa, Artificial Sequence
  Description of Artificial Sequence: Synthetic peptide
NGDPREPRRGQNRS

SEQ ID NO: 69 - protein, 14 aa, Artificial Sequence
  Description of Artificial Sequence: Synthetic peptide
NGEPREPRRGQNRS

SEQ ID NO: 70 - protein, 14 aa, Artificial Sequence
  Description of Artificial Sequence: Synthetic peptide
NGSPRDPRRGQNRS

SEQ ID NO: 71 - protein, 14 aa, Artificial Sequence
  Description of Artificial Sequence: Synthetic peptide
NGSPREPRRGQNRS

SEQ ID NO: 72 - protein, 14 aa, Artificial Sequence
  Description of Artificial Sequence: Synthetic peptide
NGDPRTPRRGQNRS

SEQ ID NO: 73 - protein, 14 aa, Artificial Sequence
  Description of Artificial Sequence: Synthetic peptide
NGEPRTPRRGQNRS

SEQ ID NO: 74 - protein, 17 aa, Artificial Sequence
  Description of Artificial Sequence: Synthetic peptide
NGDPRDPRRGQNRSARI

SEQ ID NO: 75 - protein, 17 aa, Artificial Sequence
  Description of Artificial Sequence: Synthetic peptide
NGEPRDPRRGQNRSARI

SEQ ID NO: 76 - protein, 17 aa, Artificial Sequence
  Description of Artificial Sequence: Synthetic peptide
NGDPREPRRGQNRSARI

SEQ ID NO: 77 - protein, 17 aa, Artificial Sequence
  Description of Artificial Sequence: Synthetic peptide
NGEPREPRRGQNRSARI

SEQ ID NO: 78 - protein, 17 aa, Artificial Sequence
  Description of Artificial Sequence: Synthetic peptide
NGSPRDPRRGQNRSARI

SEQ ID NO: 79 - protein, 17 aa, Artificial Sequence
  Description of Artificial Sequence: Synthetic peptide
NGSPREPRRGQNRSARI

SEQ ID NO: 80 - protein, 17 aa, Artificial Sequence
  Description of Artificial Sequence: Synthetic peptide
NGDPRTPRRGQNRSARI

SEQ ID NO: 81 - protein, 17 aa, Artificial Sequence
  Description of Artificial Sequence: Synthetic peptide
NGEPRTPRRGQNRSARI

SEQ ID NO: 82 - protein, 19 aa, Artificial Sequence
  Description of Artificial Sequence: Synthetic peptide
NGDPRDPRRGQNRSARIAK

SEQ ID NO: 83 - protein, 19 aa, Artificial Sequence
  Description of Artificial Sequence: Synthetic peptide
NGEPRDPRRGQNRSARIAK

SEQ ID NO: 84 - protein, 19 aa, Artificial Sequence
  Description of Artificial Sequence: Synthetic peptide
NGDPREPRRGQNRSARIAK

SEQ ID NO: 85 - skipped

SEQ ID NO: 86 - protein, 19 aa, Artificial Sequence
  Description of Artificial Sequence: Synthetic peptide
NGEPREPRRGQNRSARIAK

SEQ ID NO: 87 - protein, 19 aa, Artificial Sequence
  Description of Artificial Sequence: Synthetic peptide
NGSPRDPRRGQNRSARIAK

SEQ ID NO: 88 - protein, 19 aa, Artificial Sequence
  Description of Artificial Sequence: Synthetic peptide
NGSPREPRRGQNRSARIAK

SEQ ID NO: 89 - protein, 19 aa, Artificial Sequence
  Description of Artificial Sequence: Synthetic peptide
NGDPRTPRRGQNRSARIAK

SEQ ID NO: 90 - protein, 19 aa, Artificial Sequence
  Description of Artificial Sequence: Synthetic peptide
NGEPRTPRRGQNRSARIAK

SEQ ID NO: 91 - protein, 8 aa, Artificial Sequence
  Description of Artificial Sequence: Synthetic peptide
  MOD_RES (1)..(8): D-amino acid
RRRRRRRR

SEQ ID NO: 92 - protein, 16 aa, Artificial Sequence
  Description of Artificial Sequence: Synthetic peptide
  MOD_RES (1)..(8): D-amino acid
RRRRRRRRNGDPRDPR

SEQ ID NO: 93 - protein, 16 aa, Artificial Sequence
  Description of Artificial Sequence: Synthetic peptide
  MOD_RES (1)..(8): D-amino acid
RRRRRRRRNGDPREPR

SEQ ID NO: 94 - protein, 16 aa, Artificial Sequence
  Description of Artificial Sequence: Synthetic peptide
  MOD_RES (1)..(8): D-amino acid
RRRRRRRRNGEPRDPR

SEQ ID NO: 95 - protein, 16 aa, Artificial Sequence
  Description of Artificial Sequence: Synthetic peptide
  MOD_RES (1)..(8): D-amino acid
RRRRRRRRNGEPREPR

SEQ ID NO: 96 - protein, 31 aa, Artificial Sequence
  Description of Artificial Sequence: Synthetic polypeptide
  MOD_RES (1)..(8): D-amino acid
RRRRRRRRPINGDPRDPRRGQNRSARIAKQL

SEQ ID NO: 97 - protein, 31 aa, Artificial Sequence
  Description of Artificial Sequence: Synthetic polypeptide
  MOD_RES (1)..(8): D-amino acid
RRRRRRRRPINGEPRDPRRGQNRSARIAKQL

SEQ ID NO: 98 - protein, 31 aa, Artificial Sequence
  Description of Artificial Sequence: Synthetic polypeptide
  MOD_RES (1)..(8): D-amino acid
RRRRRRRRPINGDPREPRRGQNRSARIAKQL

SEQ ID NO: 99 - protein, 31 aa, Artificial Sequence
  Description of Artificial Sequence: Synthetic polypeptide
  MOD_RES (1)..(8): D-amino acid
RRRRRRRRPINGEPREPRRGQNRSARIAKQL

SEQ ID NO: 100 - protein, 31 aa, Artificial Sequence
  Description of Artificial Sequence: Synthetic polypeptide
  MOD_RES (1)..(8): D-amino acid
RRRRRRRRPFNGDPRDPRRGQNRSARIAKQL

SEQ ID NO: 101 - protein, 31 aa, Artificial Sequence
  Description of Artificial Sequence: Synthetic polypeptide
  MOD_RES (1)..(8): D-amino acid
RRRRRRRRPFNGEPRDPRRGQNRSARIAKQL

SEQ ID NO: 102 - protein, 31 aa, Artificial Sequence
  Description of Artificial Sequence: Synthetic polypeptide
  MOD_RES (1)..(8): D-amino acid
RRRRRRRRPFNGDPREPRRGQNRSARIAKQL

SEQ ID NO: 103 - protein, 31 aa, Artificial Sequence
  Description of Artificial Sequence: Synthetic polypeptide
  MOD_RES (1)..(8): D-amino acid
RRRRRRRRPFNGEPREPRRGQNRSARIAKQL

SEQ ID NO: 104 - protein, 16 aa, Artificial Sequence
  Description of Artificial Sequence: Synthetic peptide
  MOD_RES (1)..(8): D-amino acid
RRRRRRRRNGSPRDPR

SEQ ID NO: 105 - protein, 16 aa, Artificial Sequence
  Description of Artificial Sequence: Synthetic peptide
  MOD_RES (1)..(8): D-amino acid
RRRRRRRRNGSPREPR

SEQ ID NO: 106 - protein, 16 aa, Artificial Sequence
  Description of Artificial Sequence: Synthetic peptide
  MOD_RES (1)..(8): D-amino acid
RRRRRRRRNGDPRTPR

SEQ ID NO: 107 - protein, 16 aa, Artificial Sequence
  Description of Artificial Sequence: Synthetic peptide
  MOD_RES (1)..(8): D-amino acid
RRRRRRRRNGEPRTPR

SEQ ID NO: 108 - protein, 31 aa, Artificial Sequence
  Description of Artificial Sequence: Synthetic polypeptide
  MOD_RES (1)..(8): D-amino acid
RRRRRRRRPINGSPRDPRRGQNRSARIAKQL

SEQ ID NO: 109 - protein, 31 aa, Artificial Sequence
  Description of Artificial Sequence: Synthetic polypeptide
  MOD_RES (1)..(8): D-amino acid
RRRRRRRRPINGSPREPRRGQNRSARIAKQL

SEQ ID NO: 110 - protein, 31 aa, Artificial Sequence
  Description of Artificial Sequence: Synthetic polypeptide
  MOD_RES (1)..(8): D-amino acid
RRRRRRRRPINGDPRTPRRGQNRSARIAKQL

SEQ ID NO: 111 - protein, 31 aa, Artificial Sequence
  Description of Artificial Sequence: Synthetic polypeptide
  MOD_RES (1)..(8): D-amino acid
RRRRRRRRPINGEPRTPRRGQNRSARIAKQL

SEQ ID NO: 112 - protein, 31 aa, Artificial Sequence
  Description of Artificial Sequence: Synthetic polypeptide
  MOD_RES (1)..(8): D-amino acid
RRRRRRRRPFNGSPRDPRRGQNRSARIAKQL

SEQ ID NO: 113 - protein, 31 aa, Artificial Sequence
  Description of Artificial Sequence: Synthetic polypeptide
  MOD_RES (1)..(8): D-amino acid
RRRRRRRRPFNGSPREPRRGQNRSARIAKQL

SEQ ID NO: 114 - protein, 31 aa, Artificial Sequence
  Description of Artificial Sequence: Synthetic polypeptide
  MOD_RES (1)..(8): D-amino acid
RRRRRRRRPFNGDPRTPRRGQNRSARIAKQL

SEQ ID NO: 115 - protein, 31 aa, Artificial Sequence
  Description of Artificial Sequence: Synthetic polypeptide
  MOD_RES (1)..(8): D-amino acid
RRRRRRRRPFNGEPRTPRRGQNRSARIAKQL

SEQ ID NO: 116 - protein, 30 aa, Artificial Sequence
  Description of Artificial Sequence: Synthetic polypeptide
  MOD_RES (1)..(8): D-amino acid
RRRRRRRRINGDPRDPRRGQNRSARIAKQL

SEQ ID NO: 117 - protein, 30 aa, Artificial Sequence
  Description of Artificial Sequence: Synthetic polypeptide
  MOD_RES (1)..(8): D-amino acid
RRRRRRRRINGEPRDPRRGQNRSARIAKQL

SEQ ID NO: 118 - protein, 30 aa, Artificial Sequence
  Description of Artificial Sequence: Synthetic polypeptide
  MOD_RES (1)..(8): D-amino acid
RRRRRRRRINGDPREPRRGQNRSARIAKQL

SEQ ID NO: 119 - protein, 30 aa, Artificial Sequence
  Description of Artificial Sequence: Synthetic polypeptide
  MOD_RES (1)..(8): D-amino acid
RRRRRRRRINGEPREPRRGQNRSARIAKQL

SEQ ID NO: 120 - protein, 30 aa, Artificial Sequence
  Description of Artificial Sequence: Synthetic polypeptide
  MOD_RES (1)..(8): D-amino acid
RRRRRRRRFNGDPRDPRRGQNRSARIAKQL

SEQ ID NO: 121 - protein, 30 aa, Artificial Sequence
  Description of Artificial Sequence: Synthetic polypeptide
  MOD_RES (1)..(8): D-amino acid
RRRRRRRRFNGEPRDPRRGQNRSARIAKQL

SEQ ID NO: 122 - protein, 30 aa, Artificial Sequence
  Description of Artificial Sequence: Synthetic polypeptide
  MOD_RES (1)..(8): D-amino acid
RRRRRRRRFNGDPREPRRGQNRSARIAKQL

SEQ ID NO: 123 - protein, 30 aa, Artificial Sequence
  Description of Artificial Sequence: Synthetic polypeptide
  MOD_RES (1)..(8): D-amino acid
RRRRRRRRFNGEPREPRRGQNRSARIAKQL

SEQ ID NO: 124 - protein, 30 aa, Artificial Sequence
  Description of Artificial Sequence: Synthetic polypeptide
  MOD_RES (1)..(8): D-amino acid
RRRRRRRRINGSPRDPRRGQNRSARIAKQL

SEQ ID NO: 125 - protein, 30 aa, Artificial Sequence
  Description of Artificial Sequence: Synthetic polypeptide
  MOD_RES (1)..(8): D-amino acid
RRRRRRRRINGSPREPRRGQNRSARIAKQL

SEQ ID NO: 126 - protein, 30 aa, Artificial Sequence
  Description of Artificial Sequence: Synthetic polypeptide
  MOD_RES (1)..(8): D-amino acid
RRRRRRRRINGDPRTPRRGQNRSARIAKQL

SEQ ID NO: 127 - protein, 30 aa, Artificial Sequence
  Description of Artificial Sequence: Synthetic polypeptide
  MOD_RES (1)..(8): D-amino acid
RRRRRRRRINGEPRTPRRGQNRSARIAKQL

SEQ ID NO: 128 - protein, 30 aa, Artificial Sequence
  Description of Artificial Sequence: Synthetic polypeptide
  MOD_RES (1)..(8): D-amino acid
RRRRRRRRFNGSPRDPRRGQNRSARIAKQL

SEQ ID NO: 129 - protein, 30 aa, Artificial Sequence
  Description of Artificial Sequence: Synthetic polypeptide
  MOD_RES (1)..(8): D-amino acid
RRRRRRRRFNGSPREPRRGQNRSARIAKQL

SEQ ID NO: 130 - protein, 30 aa, Artificial Sequence
  Description of Artificial Sequence: Synthetic polypeptide
  MOD_RES (1)..(8): D-amino acid
RRRRRRRRFNGDPRTPRRGQNRSARIAKQL

SEQ ID NO: 131 - protein, 30 aa, Artificial Sequence
  Description of Artificial Sequence: Synthetic polypeptide
  MOD_RES (1)..(8): D-amino acid
RRRRRRRRFNGEPRTPRRGQNRSARIAKQL

SEQ ID NO: 132 - protein, 29 aa, Artificial Sequence
  Description of Artificial Sequence: Synthetic peptide
  MOD_RES (1)..(8): D-amino acid
RRRRRRRRNGDPRDPRRGQNRSARIAKQL

SEQ ID NO: 133 - protein, 29 aa, Artificial Sequence
  Description of Artificial Sequence: Synthetic peptide
  MOD_RES (1)..(8): D-amino acid
RRRRRRRRNGEPRDPRRGQNRSARIAKQL

SEQ ID NO: 134 - protein, 29 aa, Artificial Sequence
  Description of Artificial Sequence: Synthetic peptide
  MOD_RES (1)..(8): D-amino acid
RRRRRRRRNGDPREPRRGQNRSARIAKQL

SEQ ID NO: 135 - protein, 29 aa, Artificial Sequence
  Description of Artificial Sequence: Synthetic peptide
  MOD_RES (1)..(8): D-amino acid
RRRRRRRRNGEPREPRRGQNRSARIAKQL

SEQ ID NO: 136 - protein, 29 aa, Artificial Sequence
  Description of Artificial Sequence: Synthetic peptide
  MOD_RES (1)..(8): D-amino acid
RRRRRRRRNGSPRDPRRGQNRSARIAKQL

SEQ ID NO: 137 - protein, 29 aa, Artificial Sequence
  Description of Artificial Sequence: Synthetic peptide
  MOD_RES (1)..(8): D-amino acid
RRRRRRRRNGSPREPRRGQNRSARIAKQL

SEQ ID NO: 138 - protein, 29 aa, Artificial Sequence
  Description of Artificial Sequence: Synthetic peptide
  MOD_RES (1)..(8): D-amino acid
RRRRRRRRNGDPRTPRRGQNRSARIAKQL

SEQ ID NO: 139 - protein, 29 aa, Artificial Sequence
  Description of Artificial Sequence: Synthetic peptide
  MOD_RES (1)..(8): D-amino acid
RRRRRRRRNGEPRTPRRGQNRSARIAKQL

SEQ ID NO: 140 - protein, 29 aa, Artificial Sequence
  Description of Artificial Sequence: Synthetic peptide
  MOD_RES (1)..(8): D-amino acid
RRRRRRRRNGDPRDPRRGQNRSARIAKQL

SEQ ID NO: 141 - protein, 29 aa, Artificial Sequence
  Description of Artificial Sequence: Synthetic peptide
  MOD_RES (1)..(8): D-amino acid
RRRRRRRRNGEPRDPRRGQNRSARIAKQL

SEQ ID NO: 142 - protein, 29 aa, Artificial Sequence
  Description of Artificial Sequence: Synthetic peptide
  MOD_RES (1)..(8): D-amino acid
RRRRRRRRNGDPREPRRGQNRSARIAKQL

SEQ ID NO: 143 - protein, 29 aa, Artificial Sequence
  Description of Artificial Sequence: Synthetic peptide
  MOD_RES (1)..(8): D-amino acid
RRRRRRRRNGEPREPRRGQNRSARIAKQL

SEQ ID NO: 144 - protein, 29 aa, Artificial Sequence
  Description of Artificial Sequence: Synthetic peptide
  MOD_RES (1)..(8): D-amino acid
RRRRRRRRNGSPRDPRRGQNRSARIAKQL

SEQ ID NO: 145 - protein, 29 aa, Artificial Sequence
  Description of Artificial Sequence: Synthetic peptide
  MOD_RES (1)..(8): D-amino acid
RRRRRRRRNGSPREPRRGQNRSARIAKQL

SEQ ID NO: 146 - protein, 29 aa, Artificial Sequence
  Description of Artificial Sequence: Synthetic peptide
  MOD_RES (1)..(8): D-amino acid
RRRRRRRRNGDPRTPRRGQNRSARIAKQL

SEQ ID NO: 147 - protein, 29 aa, Artificial Sequence
  Description of Artificial Sequence: Synthetic peptide
  MOD_RES (1)..(8): D-amino acid
RRRRRRRRNGEPRTPRRGQNRSARIAKQL

SEQ ID NO: 148 - protein, 18 aa, Artificial Sequence
  Description of Artificial Sequence: Synthetic peptide
  MOD_RES (1)..(8): D-amino acid
RRRRRRRRNGDPRDPRRG

SEQ ID NO: 149 - protein, 18 aa, Artificial Sequence
  Description of Artificial Sequence: Synthetic peptide
  MOD_RES (1)..(8): D-amino acid
RRRRRRRRNGEPRDPRRG

SEQ ID NO: 150 - protein, 18 aa, Artificial Sequence
  Description of Artificial Sequence: Synthetic peptide
  MOD_RES (1)..(8): D-amino acid
RRRRRRRRNGDPREPRRG

SEQ ID NO: 151 - protein, 18 aa, Artificial Sequence
  Description of Artificial Sequence: Synthetic peptide
  MOD_RES (1)..(8): D-amino acid
RRRRRRRRNGEPREPRRG

SEQ ID NO: 152 - protein, 18 aa, Artificial Sequence
  Description of Artificial Sequence: Synthetic peptide
  MOD_RES (1)..(8): D-amino acid
RRRRRRRRNGSPRDPRRG

SEQ ID NO: 153 - protein, 18 aa, Artificial Sequence
  Description of Artificial Sequence: Synthetic peptide
  MOD_RES (1)..(8): D-amino acid
RRRRRRRRNGSPREPRRG

SEQ ID NO: 154 - protein, 18 aa, Artificial Sequence
  Description of Artificial Sequence: Synthetic peptide
  MOD_RES (1)..(8): D-amino acid
RRRRRRRRNGDPRTPRRG

SEQ ID NO: 155 - protein, 18 aa, Artificial Sequence
  Description of Artificial Sequence: Synthetic peptide
  MOD_RES (1)..(8): D-amino acid
RRRRRRRRNGEPRTPRRG

SEQ ID NO: 156 - protein, 22 aa, Artificial Sequence
  Description of Artificial Sequence: Synthetic peptide
  MOD_RES (1)..(8): D-amino acid
RRRRRRRRNGDPRDPRRGQNRS

SEQ ID NO: 157 - protein, 22 aa, Artificial Sequence
  Description of Artificial Sequence: Synthetic peptide
  MOD_RES (1)..(8): D-amino acid
RRRRRRRRNGEPRDPRRGQNRS

SEQ ID NO: 158 - protein, 22 aa, Artificial Sequence
  Description of Artificial Sequence: Synthetic peptide
  MOD_RES (1)..(8): D-amino acid
RRRRRRRRNGDPREPRRGQNRS

SEQ ID NO: 159 - protein, 22 aa, Artificial Sequence
  Description of Artificial Sequence: Synthetic peptide
  MOD_RES (1)..(8): D-amino acid
RRRRRRRRNGEPREPRRGQNRS

SEQ ID NO: 160 - protein, 22 aa, Artificial Sequence
  Description of Artificial Sequence: Synthetic peptide
  MOD_RES (1)..(8): D-amino acid
RRRRRRRRNGSPRDPRRGQNRS

SEQ ID NO: 161 - protein, 22 aa, Artificial Sequence
  Description of Artificial Sequence: Synthetic peptide
  MOD_RES (1)..(8): D-amino acid
RRRRRRRRNGSPREPRRGQNRS

SEQ ID NO: 162 - protein, 22 aa, Artificial Sequence
  Description of Artificial Sequence: Synthetic peptide
  MOD_RES (1)..(8): D-amino acid
RRRRRRRRNGDPRTPRRGQNRS

SEQ ID NO: 163 - protein, 22 aa, Artificial Sequence
  Description of Artificial Sequence: Synthetic peptide
  MOD_RES (1)..(8): D-amino acid
RRRRRRRRNGEPRTPRRGQNRS

SEQ ID NO: 164 - protein, 25 aa, Artificial Sequence
  Description of Artificial Sequence: Synthetic peptide
  MOD_RES (1)..(8): D-amino acid
RRRRRRRRNGDPRDPRRGQNRSARI

SEQ ID NO: 165 - protein, 25 aa, Artificial Sequence
  Description of Artificial Sequence: Synthetic peptide
  MOD_RES (1)..(8): D-amino acid
RRRRRRRRNGEPRDPRRGQNRSARI

SEQ ID NO: 166 - protein, 25 aa, Artificial Sequence
  Description of Artificial Sequence: Synthetic peptide
  MOD_RES (1)..(8): D-amino acid
RRRRRRRRNGDPREPRRGQNRSARI

SEQ ID NO: 167 - protein, 25 aa, Artificial Sequence
  Description of Artificial Sequence: Synthetic peptide
  MOD_RES (1)..(8): D-amino acid
RRRRRRRRNGEPREPRRGQNRSARI

SEQ ID NO: 168 - protein, 25 aa, Artificial Sequence
  Description of Artificial Sequence: Synthetic peptide
  MOD_RES (1)..(8): D-amino acid
RRRRRRRRNGSPRDPRRGQNRSARI

SEQ ID NO: 169 - protein, 25 aa, Artificial Sequence
  Description of Artificial Sequence: Synthetic peptide
  MOD_RES (1)..(8): D-amino acid
RRRRRRRRNGSPREPRRGQNRSARI

SEQ ID NO: 170 - protein, 25 aa, Artificial Sequence
  Description of Artificial Sequence: Synthetic peptide
  MOD_RES (1)..(8): D-amino acid
RRRRRRRRNGDPRTPRRGQNRSARI

SEQ ID NO: 171 - protein, 25 aa, Artificial Sequence
  Description of Artificial Sequence: Synthetic peptide
  MOD_RES (1)..(8): D-amino acid
RRRRRRRRNGEPRTPRRGQNRSARI

SEQ ID NO: 172 - protein, 27 aa, Artificial Sequence
  Description of Artificial Sequence: Synthetic peptide
  MOD_RES (1)..(8): D-amino acid
RRRRRRRRNGDPRDPRRGQNRSARIAK

SEQ ID NO: 173 - protein, 27 aa, Artificial Sequence
  Description of Artificial Sequence: Synthetic peptide
  MOD_RES (1)..(8): D-amino acid
RRRRRRRRNGEPRDPRRGQNRSARIAK

SEQ ID NO: 174 - protein, 27 aa, Artificial Sequence
  Description of Artificial Sequence: Synthetic peptide
  MOD_RES (1)..(8): D-amino acid
RRRRRRRRNGDPREPRRGQNRSARIAK

SEQ ID NO: 175 - protein, 27 aa, Artificial Sequence
  Description of Artificial Sequence: Synthetic peptide
  MOD_RES (1)..(8): D-amino acid
RRRRRRRRNGEPREPRRGQNRSARIAK

SEQ ID NO: 176 - protein, 27 aa, Artificial Sequence
  Description of Artificial Sequence: Synthetic peptide
  MOD_RES (1)..(8): D-amino acid
RRRRRRRRNGSPRDPRRGQNRSARIAK

SEQ ID NO: 177 - protein, 27 aa, Artificial Sequence
  Description of Artificial Sequence: Synthetic peptide
  MOD_RES (1)..(8): D-amino acid
RRRRRRRRNGSPREPRRGQNRSARIAK

SEQ ID NO: 178 - protein, 27 aa, Artificial Sequence
  Description of Artificial Sequence: Synthetic peptide
  MOD_RES (1)..(8): D-amino acid
RRRRRRRRNGDPRTPRRGQNRSARIAK

SEQ ID NO: 179 - protein, 27 aa, Artificial Sequence
  Description of Artificial Sequence: Synthetic peptide
  MOD_RES (1)..(8): D-amino acid
RRRRRRRRNGEPRTPRRGQNRSARIAK

SEQ ID NO: 180 - protein, 16 aa, Artificial Sequence
  Description of Artificial Sequence: Synthetic peptide
  MOD_RES (1)..(8): D-amino acid
  NON_CONS (8)..(9): One or more peptidase cleavage sites
  See specification as filed for detailed description of substitutions and preferred embodiments
RRRRRRRRNGDPRDPR

SEQ ID NO: 181 - protein, 19 aa, Artificial Sequence
  Description of Artificial Sequence: Synthetic peptide
  MOD_RES (1)..(11): D-amino acid
RRRRRRRRAFKNGDPRDPR

SEQ ID NO: 182 - DNA, 58 bp, Artificial Sequence
  Description of Artificial Sequence: Synthetic oligonucleotide
ccgggtgcgctcttgaggttgtaatctcgagattacaacctcaagagcgcactttttg

SEQ ID NO: 183 - DNA, 58 bp, Artificial Sequence
  Description of Artificial Sequence: Synthetic oligonucleotide
ccggcagagatcgtgtattgagattctcgagaatctcaatacacgatctctgtttttg

SEQ ID NO: 184 - DNA, 58 bp, Artificial Sequence
  Description of Artificial Sequence: Synthetic oligonucleotide
ccgggacttctactcgaacacgaatctcgagattcgtgttcgagtagaagtctttttg

SEQ ID NO: 185 - DNA, 58 bp, Artificial Sequence
  Description of Artificial Sequence: Synthetic oligonucleotide
ccggtgatgaagcacaccgattaaactcgagtttaatcggtgtgcttcatcatttttg

SEQ ID NO: 186 - DNA, 57 bp, Artificial Sequence
  Description of Artificial Sequence: Synthetic oligonucleotide
ccgggcggtttatcaagagctataactcgagttatagctcttgataaaccgcttttt

SEQ ID NO: 187 - DNA, 56 bp, Artificial Sequence
  Description of Artificial Sequence: Synthetic oligonucleotide
ccggcggattgaggagaaacgtaaactcgagtttacgtttctcctcaatccgtttt

SEQ ID NO: 188 - DNA, 56 bp, Artificial Sequence; identical to SEQ ID NO: 187
  Description of Artificial Sequence: Synthetic oligonucleotide

SEQ ID NO: 189 - DNA, 57 bp, Artificial Sequence
  Description of Artificial Sequence: Synthetic oligonucleotide
ccggcaccatcaactatgatgagttctcgagaactcatcatagttgatggtgttttt

SEQ ID NO: 190 - DNA, 57 bp, Artificial Sequence
  Description of Artificial Sequence: Synthetic oligonucleotide
ccgggccttaatagtagggtaagttctcgagaacttaccctactattaaggcttttt

SEQ ID NO: 191 - DNA, 57 bp, Artificial Sequence
  Description of Artificial Sequence: Synthetic oligonucleotide
ccggatgactggcctcgagatgtactcgagtacatctcgaggccagtcatctttttg

SEQ ID NO: 192 - DNA, 57 bp, Artificial Sequence
  Description of Artificial Sequence: Synthetic oligonucleotide
ccggacagttcgtgaggtggctttactcgagtaaagccacctcacgaactgtttttt

SEQ ID NO: 193 - DNA, 57 bp, Artificial Sequence
  Description of Artificial Sequence: Synthetic oligonucleotide
ccggaggacatatctggacaaggcactcgagtgccttgtccagatatgtcctttttt

SEQ ID NO: 194 - DNA, 56 bp, Artificial Sequence
  Description of Artificial Sequence: Synthetic oligonucleotide
ccggtctggagtgttggctgcatatctcgagatatgcagccaacactccagatttt

SEQ ID NO: 195 - DNA, 59 bp, Artificial Sequence
  Description of Artificial Sequence: Synthetic oligonucleotide
ccggcatgagatgttcctatcttaactcgagttaagataggaacatctcatgttttttg

SEQ ID NO: 196 - DNA, 59 bp, Artificial Sequence
  Description of Artificial Sequence: Synthetic oligonucleotide
ccgggagaagtttgtaacagatatcctcgaggatatctgttacaaacttctcttttttg

SEQ ID NO: 197 - DNA, 59 bp, Artificial Sequence
  Description of Artificial Sequence: Synthetic oligonucleotide
ccgggcagatgacgtatgggatatcctcgaggatatcccatacgtcatctgcttttttg

SEQ ID NO: 198 - DNA, 59 bp, Artificial Sequence
  Description of Artificial Sequence: Synthetic oligonucleotide
ccggccagatactttctttactaagctcgagcttagtaaagaaagtatctggttttttg

SEQ ID NO: 199 - DNA, 59 bp, Artificial Sequence
  Description of Artificial Sequence: Synthetic oligonucleotide
ccgggctgctcacaagttctatttcctcgaggaaatagaacttgtgagcagcttttttg

SEQ ID NO: 200 - DNA, 58 bp, Artificial Sequence
  Description of Artificial Sequence: Synthetic oligonucleotide
ccgggctggttcatatcttgaacatctcgagatgttcaagatatgaaccagctttttg

SEQ ID NO: 201 - DNA, 57 bp, Artificial Sequence
  Description of Artificial Sequence: Synthetic oligonucleotide
ccgggctggttcatatcttgaacatctcgagatgttcaagatatgaaccagcttttt

SEQ ID NO: 202 - DNA, 57 bp, Artificial Sequence
  Description of Artificial Sequence: Synthetic oligonucleotide
ccggccagccaagaagagtgaagaactcgagttcttcactcttcttggctggttttt

SEQ ID NO: 203 - DNA, 20 bp, Artificial Sequence
  Description of Artificial Sequence: Synthetic primer
acccagaagactgtggatgg

SEQ ID NO: 204 - DNA, 20 bp, Artificial Sequence
  Description of Artificial Sequence: Synthetic primer
ttcagctcagggatgacctt

SEQ ID NO: 205 - DNA, 20 bp, Artificial Sequence
  Description of Artificial Sequence: Synthetic primer
gacctctatgccaacacagt

SEQ ID NO: 206 - DNA, 20 bp, Artificial Sequence
  Description of Artificial Sequence: Synthetic primer
agtacttgcgctcaggagga

SEQ ID NO: 207 - DNA, 19 bp, Artificial Sequence
  Description of Artificial Sequence: Synthetic primer
gaggccaagccctggtatg

SEQ ID NO: 208 - DNA, 19 bp, Artificial Sequence
  Description of Artificial Sequence: Synthetic primer
cgggccgattgatctcagc

SEQ ID NO: 209 - DNA, 21 bp, Artificial Sequence
  Description of Artificial Sequence: Synthetic primer
tgctgtaccaagagtttgctc

SEQ ID NO: 210 - DNA, 23 bp, Artificial Sequence
  Description of Artificial Sequence: Synthetic primer
cgcacacagacaactttttcttt

SEQ ID NO: 211 - DNA, 20 bp, Artificial Sequence
  Description of Artificial Sequence: Synthetic primer
tttctgcttttgcattcgtg

SEQ ID NO: 212 - DNA, 20 bp, Artificial Sequence
  Description of Artificial Sequence: Synthetic primer
ggaagcaaccctcctaaacc

SEQ ID NO: 213 - DNA, 20 bp, Artificial Sequence
  Description of Artificial Sequence: Synthetic primer
ggtgccgactacaagcgaat

SEQ ID NO: 214 - DNA, 20 bp, Artificial Sequence
  Description of Artificial Sequence: Synthetic primer
agccctcagcctgacatgtc

SEQ ID NO: 215 - DNA, 20 bp, Artificial Sequence
  Description of Artificial Sequence: Synthetic primer
aacagccaccagtgagcttc

SEQ ID NO: 216 - DNA, 20 bp, Artificial Sequence
  Description of Artificial Sequence: Synthetic primer
gaaagcttgcctcaatcctg

SEQ ID NO: 217 - DNA, 19 bp, Artificial Sequence
  Description of Artificial Sequence: Synthetic primer
tgacaacgacatcaacatc

SEQ ID NO: 218 - DNA, 19 bp, Artificial Sequence
  Description of Artificial Sequence: Synthetic primer
gtgaccagagacaatgcag

SEQ ID NO: 219 - DNA, 19 bp, Artificial Sequence
  Description of Artificial Sequence: Synthetic primer
gtctcagctgctcgacacg

SEQ ID NO: 220 - DNA, 20 bp, Artificial Sequence
  Description of Artificial Sequence: Synthetic primer
ttttgcactgtgcgcttaaa

SEQ ID NO: 221 - DNA, 19 bp, Artificial Sequence
  Description of Artificial Sequence: Synthetic primer
tcctgagctgcgcctacag

SEQ ID NO: 222 - DNA, 20 bp, Artificial Sequence
  Description of Artificial Sequence: Synthetic primer
gcagggcaagtccacatctt

SEQ ID NO: 223 - DNA, 21 bp, Artificial Sequence
  Description of Artificial Sequence: Synthetic primer
tgtggcctgatgttggataac

SEQ ID NO: 224 - DNA, 23 bp, Artificial Sequence
  Description of Artificial Sequence: Synthetic primer
ggtgacgaatgtgcaaatctact

SEQ ID NO: 225 - DNA, 21 bp, Artificial Sequence
  Description of Artificial Sequence: Synthetic primer
acgcagcaatcgtgcattttg

SEQ ID NO: 226 - DNA, 22 bp, Artificial Sequence
  Description of Artificial Sequence: Synthetic primer
cctataacgaggtcactgacgg

SEQ ID NO: 227 - DNA, 20 bp, Artificial Sequence
  Description of Artificial Sequence: Synthetic primer
tgagctgctgctactggaga

SEQ ID NO: 228 - DNA, 20 bp, Artificial Sequence
  Description of Artificial Sequence: Synthetic primer
tcccggcaaaaacaaataag

SEQ ID NO: 229 - DNA, 20 bp, Artificial Sequence
  Description of Artificial Sequence: Synthetic primer
tcaagcctgtcctgacacag

SEQ ID NO: 230 - DNA, 20 bp, Artificial Sequence
  Description of Artificial Sequence: Synthetic primer
caggtccacagtcttgctca

SEQ ID NO: 231 - DNA, 20 bp, Artificial Sequence
  Description of Artificial Sequence: Synthetic primer
atacgacttggcgaatcagg

SEQ ID NO: 232 - DNA, 20 bp, Artificial Sequence
  Description of Artificial Sequence: Synthetic primer
gagcgcttcttggtgtaagg

SEQ ID NO: 233 - DNA, 21 bp, Artificial Sequence
  Description of Artificial Sequence: Synthetic primer
aggttgtctcctgcgacttca

SEQ ID NO: 234 - DNA, 22 bp, Artificial Sequence
  Description of Artificial Sequence: Synthetic primer
gggtggtccagggtttcttact

SEQ ID NO: 235 - DNA, 20 bp, Artificial Sequence
  Description of Artificial Sequence: Synthetic primer
aatgctgcccttcagatcac

SEQ ID NO: 236 - DNA, 20 bp, Artificial Sequence
  Description of Artificial Sequence: Synthetic primer
ataaccctcggcctgacata

SEQ ID NO: 237 - DNA, 18 bp, Artificial Sequence
  Description of Artificial Sequence: Synthetic primer
gcactgcctcctggtcac

SEQ ID NO: 238 - DNA, 20 bp, Artificial Sequence
  Description of Artificial Sequence: Synthetic primer
tgcctctgctctcttcacag

SEQ ID NO: 239 - DNA, 20 bp, Artificial Sequence
  Description of Artificial Sequence: Synthetic primer
tgagctgctgctactggaga

SEQ ID NO: 240 - DNA, 20 bp, Artificial Sequence
  Description of Artificial Sequence: Synthetic primer
tcccggcaaaaacaaataag

SEQ ID NO: 241 - DNA, 24 bp, Artificial Sequence
  Description of Artificial Sequence: Synthetic primer
tgatggtggcttgagatgttggga

SEQ ID NO: 242 - DNA, 27 bp, Artificial Sequence
  Description of Artificial Sequence: Synthetic primer
tgaatctcatcaacaaactcctgaccc

SEQ ID NO: 243 - DNA, 20 bp, Artificial Sequence
  Description of Artificial Sequence: Synthetic primer
tgtcagccaagttcaagctg

SEQ ID NO: 244 - DNA, 22 bp, Artificial Sequence
  Description of Artificial Sequence: Synthetic primer
atcttccgaactttctccaggg

SEQ ID NO: 245 - DNA, 20 bp, Artificial Sequence
  Description of Artificial Sequence: Synthetic primer
gaaaggcaatcccagaaagg

SEQ ID NO: 246 - DNA, 20 bp, Artificial Sequence
  Description of Artificial Sequence: Synthetic primer
tctagctaaaagccggttgc

SEQ ID NO: 247 - DNA, 27 bp, Artificial Sequence
  Description of Artificial Sequence: Synthetic primer
ggacaccaacactagatacctaaactg

SEQ ID NO: 248 - DNA, 18 bp, Artificial Sequence
  Description of Artificial Sequence: Synthetic primer
ctgcccaaggcagcaaat

SEQ ID NO: 249 - DNA, 20 bp, Artificial Sequence
  Description of Artificial Sequence: Synthetic primer
ccagcagaacttgggaaagg

SEQ ID NO: 250 - DNA, 20 bp, Artificial Sequence
  Description of Artificial Sequence: Synthetic primer
gcgaatgccagaaaagaaaa

SEQ ID NO: 251 - DNA, 20 bp, Artificial Sequence
  Description of Artificial Sequence: Synthetic primer
caggtagtacgcacctggag

SEQ ID NO: 252 - DNA, 20 bp, Artificial Sequence
  Description of Artificial Sequence: Synthetic primer
ctgggacaggaaaagggagt

SEQ ID NO: 253 - DNA, 19 bp, Artificial Sequence
  Description of Artificial Sequence: Synthetic primer
gcctaagcgggactaggag

SEQ ID NO: 254 - DNA, 18 bp, Artificial Sequence
  Description of Artificial Sequence: Synthetic primer
ctgccacgagctgcagag

SEQ ID NO: 255 - DNA, 20 bp, Artificial Sequence
  Description of Artificial Sequence: Synthetic oligonucleotide
ggtcaggaaagtccaacgcc

SEQ ID NO: 256 - DNA, 20 bp, Artificial Sequence
  Description of Artificial Sequence: Synthetic probe
ggtcaggaaagtccaacgcc

SEQ ID NO: 257 - DNA, 20 bp, Artificial Sequence
  Description of Artificial Sequence: Synthetic probe
ggtcattccctgaacacgcc

SEQ ID NO: 258 - protein, 5 aa, Unknown
  Description of Unknown: FxxxV conserved motif sequence
FQVTV

SEQ ID NO: 259 - protein, 19 aa, Homo sapiens
LNAVRLCFQVTVRDPAGRP

SEQ ID NO: 260 - protein, 19 aa, Mus sp.
LNAVRLCFQVTVRDPAGRP

SEQ ID NO: 261 - protein, 19 aa, Bos sp.
LNAVRLCFQVTVRDPAGRP

SEQ ID NO: 262 - protein, 19 aa, Sus sp.
LNAVRLCFQVTVRDPAGRP

SEQ ID NO: 263 - protein, 19 aa, Canis sp.
LNAVRLCFQVTVRDPAGRP

SEQ ID NO: 264 - protein, 19 aa, Homo sapiens
LAFIEELFSLMVVNRLTEE

SEQ ID NO: 265 - protein, 23 aa, Homo sapiens
PINGSPRTPRRGQNRSARIAKQL

SEQ ID NO: 266 - protein, 23 aa, Homo sapiens
PINGSPRTPRRGQNRSARIAKQL

SEQ ID NO: 267 - protein, 19 aa, Homo sapiens
LNAVRLCFQVTVRDPSGRP

SEQ ID NO: 268 - protein, 355 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQR

SEQ ID NO: 269 - protein, 331 aa, Homo sapiens
MFEDKPHAEGAAVVAAAGEALQALCQELNLDEGSAAEALDDFTAIRGNYSLEGEVTHWLACSLYVACRKSIIPTVGKGIMEGNCVSLTRILRSAKLSLIQFFSKMKKWMDMSNLPQEFRERIERLERNFEVSTVIFKKYEPIFLDIFQNPYEEPPKLPRSRKQRRIPCSVKDLFNFCWTLFVYTKGNFRMIGDDLVNSYHLLLCCLDLIFANAIMCPNRQDLLNPSFKGLPSDFHTADFTASEEPPCIIAVLCELHDGLLVEAKGIKEHYFKPYISKLFDRKILKGECLLDLSSFTDNSKAVNKEYEEYVLTVGDFDERIFLGADAEEEIG

SEQ ID NO: 270 - protein, 363 aa, Homo sapiens
MPSGGDQSPPPPPPPPAAAASDEEEEDDGEAEDAAPPAESPTPQIQQRFDELCSRLNMDEAARAEAWDSYRSMSESYTLEGNDLHWLACALYVACRKSVPTVSKGTVEGNYVSLTRILKCSEQSLIEFFNKMKKWEDMANLPPHFRERTERLERNFTVSAVIFKKYEPIFQDIFKYPQEEQPRQQRGRKQRRQPCTVSEIFHFCWVLFIYAKGNFPMISDDLVNSYHLLLCALDLVYGNALQCSNRKELVNPNFKGLSEDFHAKDSKPSSDPPCIIEKLCSLHDGLVLEAKGIKEHFWKPYIRKLYEKKLLKGKEENLTGFLEPGNFGESFKAINKAYEEYVLSVGNLDERIFLGEDAEEEIG

SEQ ID NO: 271 - protein, 23 aa, Mus sp.
PINGSPRTPRRGQNRSARIAKQL

SEQ ID NO: 272 - protein, 23 aa, Bos sp.
PINGSPRTPRRGQNRSARIAKQL

SEQ ID NO: 273 - protein, 23 aa, Sus sp.
PINGSPRTPRRGQNRSARIAKQL

SEQ ID NO: 274 - protein, 23 aa, Canis sp.
PFNGSPRTPRRGQNRSARIAKQL

SEQ ID NO: 275 - protein, 19 aa, Unknown
  Description of Unknown: EID1 sequence
LAFIEELFSLMVVNRLTEE

SEQ ID NO: 276 - DNA, 20 bp, Homo sapiens
ggtccgggactctccgcgga

SEQ ID NO: 277 - DNA, 20 bp, Homo sapiens
cgggtgggatttccctaccc

SEQ ID NO: 278 - DNA, 20 bp, Homo sapiens
gcgcagggaagttcccgaac

SEQ ID NO: 279 - DNA, 20 bp, Homo sapiens
ggtcaggaaagtccaacgcc

SEQ ID NO: 280 - DNA, 20 bp, Artificial Sequence
  Description of Artificial Sequence: Synthetic oligonucleotide
ggtcattccctgaacacgcc

SEQ ID NO: 281 - protein, 19 aa, Unknown
  Description of Unknown: Mammalian expression vector sequence
LNAVRLCFQVTVRDPSGRP

SEQ ID NO: 282 - protein, 19 aa, Artificial Sequence
  Description of Artificial Sequence: Synthetic peptide
LNAVALCFQVTVADPSGAP

SEQ ID NO: 283 - protein, 19 aa, Artificial Sequence
  Description of Artificial Sequence: Synthetic peptide
LNAVDLCFQVTVDDPSGDP

SEQ ID NO: 284 - protein, 19 aa, Artificial Sequence
  Description of Artificial Sequence: Synthetic peptide
LAFIAALFSLMVVNRLTAA

SEQ ID NO: 285 - protein, 19 aa, Artificial Sequence
  Description of Artificial Sequence: Synthetic peptide
LAFIRRLFSLMVVNRLTRR

SEQ ID NO: 286 - protein, 5 aa, Unknown
  Description of Unknown: FxxxV conserved motif sequence
FSLMV

SEQ ID NO: 287 - protein, 19 aa, Unknown
  Description of Unknown: Mammalian expression vector sequence
LAFIEELFSLMVVNRLTEE

(19) United States
(12) Patent Application Publication
(10) Pub. No.: US 2021/0401931 A1
(43) Pub. Date: Dec. 30, 2021

(54) METHODS AND MATERIALS FOR TREATING CANCER

(71) Applicant: Mayo Foundation for Medical Education and Research, Rochester, MN (US)

(72) Inventor: Haojie Huang, Rochester, MN (US)

(21) Appl. No.: 16/975,502

(22) PCT Filed: Feb. 28, 2019
(86) PCT No.: PCT/US19/20073

§ 371 Date: Aug. 25, 2020

Related U.S. Application Data
(60) Provisional application No. 62/636,734, filed on Feb. 28, 2018.
(60) Provisional application No. 62/758,429, filed on Nov. 9, 2018.

(51) Int. Cl. A61K 38/17; A61P 35/00; A61K 31/4745; A61K 31/337; A61K 31/7068; C07K 14/47; C12N 15/86; A61N 5/10

(52) U.S. Cl. CPC A61K 38/1709; A61P 35/00; A61K 31/4745; A61K 31/337; A61N 2005/1098; C07K 14/4703; C12N 15/86; A61N 5/10; C12N 2740/15043; A61K 31/7068

(57) ABSTRACT

This document provides materials and methods for treating cancer (e.g., PD-L1⁺ cancers). For example, methods and materials for using compositions (e.g., compositions containing a small bioactive S249/T252 phospho-mimicking polypeptide of an RB polypeptide) to reduce PD-L1 expression within cancer cells are provided. In addition, methods and materials for using compositions (e.g., compositions containing a small bioactive S249/T252 phospho-mimicking polypeptide of an RB polypeptide) in combination with other cancer treatment methods or agents to increase the effectiveness exhibited against the cancer within a mammal (e.g., a human) are provided.

### CROSS-REFERENCE TO RELATED APPLICATIONS

This application claims priority to U.S. Application Ser. Nos. 62/758,429, filed on Nov. 9, 2018 and 62/636,734, filed on Feb. 28, 2018. The disclosures of the prior applications are considered part of the disclosure of this application, and are incorporated in its entirety into this application.

### BACKGROUND

### 1. Technical Field

This document relates to materials and methods for treating cancer (e.g., PD-L1⁺ cancers). For example, this document provides methods and materials for using compositions (e.g., compositions containing a small bioactive S249/T252 phospho-mimicking polypeptide of an RB polypeptide) to reduce PD-L1 expression within cancer cells. This document also provides methods and materials for using compositions (e.g., compositions containing a small bioactive S249/T252 phospho-mimicking polypeptide of an RB polypeptide) in combination with other cancer treatment methods or agents to increase the effectiveness exhibited against the cancer within a mammal (e.g., a human).

### 2. Background Information

Evasion of immune surveillance is a hallmark of human cancers (Hanahan and Weinberg, Cell, 144:646-674 (2011)). Neoantigens generated by cancerous cells are potentially recognizable by the immune system. However, tumors can often escape from immune attack via various distinct mechanisms, including the aberrant activation of immune checkpoints that terminates immune responses (Sharma and Allison, Cell, 161:205-214 (2015)).

Programmed death 1 (PD-1) is a major immune checkpoint inhibitory molecule expressed in activated T cells (Ishida et al., EMBO J 11:3887-3895 (1992)). Expression of its ligand PD-L1 (also known as B7-H1) in cancer cells triggers the engagement of PD-L1 with the PD-1 receptor on T cells, causing T cell apoptosis and decreased cytotoxic T cell function (Dong et al., Nat. Med., 8:793-800 (2002); and Dong et al., Nat. Med., 5:1365-1369 (1999)). Given that blockade of the PD-1/PD-L1 interaction can reactivate T-cell responses (Topalian et al., Curr. Opin. Immunol., 24:207-212 (2012)), several antibodies against PD-1 and PD-L1 molecules have been approved for treatment of human cancers in clinic. Although the PD-1/PD-L1 blockade can improve patient progress-free survival, the response rate among all patients is relatively low. Increasing evidence indicates that response to immune checkpoint therapies appears to highly correlate with PD-L1 expression in tumor cells (Topalian et al., N. Engl. J. Med., 366:2443-2454 (2012)).

The retinoblastoma protein RB is a well-studied tumor suppressor. It is a multi-functional protein that regulates a number of critical cellular activities, which include late G1 restriction point control and cell cycle progression, DNA damage response checkpoint activation, cell cycle exit and senescence, and differentiation (Manning and Dyson, Nat. Rev. Cancer, 12:220-226 (2012)). RB, along with its homolog proteins p107 and p130, belongs to the “pocket” protein family, which plays important roles in regulation of cell proliferation. It is generally accepted that RB protein exists in two function-related statuses, one is un- or hypo-phosphorylated state and the other is hyper-phosphorylated state (Narasimha et al., Elife 3 (2014)). In the underphosphorylated form, RB interacts through the pocket domain with E2F transcription factors and represses E2F transcription factors and thereby blocks G1/S transition. During the late G1 or upon mitogen stimulation, RB becomes phosphorylated at multiple sites mediated by CYCLIN D/CDK4/6 and CYCLIN E/CDK2 complexes, which causes disassociation of RB from E2F factors and allows cell-cycle progression (Sherr and Roberts, Genes Dev., 18:2699-2711 (2004)). Thus, it is well established that RB functions as a tumor suppressor by repressing the activities of E2F transcription factors and this function is abolished due to CDK-mediated phosphorylation. However, increasing evidence suggests that there exists E2F-independent tumor suppressor function of RB that can promote cancer progression, although the signaling pathways responsible for such function remains to be identified (Sun et al., Proc. Natl. Acad. Sci. USA, 108:704-709 (2011)).

### SUMMARY

This document provides materials and methods for treating cancer (e.g., PD-L1⁺ cancers). For example, this document provides methods and materials for using compositions (e.g., compositions containing a small bioactive S249/T252 phospho-mimicking polypeptide of an RB polypeptide) to reduce PD-L1 expression within cancer cells. This document also provides methods and materials for using compositions (e.g., compositions containing a small bioactive S249/T252 phospho-mimicking polypeptide of an RB polypeptide) in combination with other cancer treatment methods or agents to increase the effectiveness exhibited against the cancer within a mammal (e.g., a human).

As demonstrated herein, RB protein directly binds to an FxxxV motif in the DNA binding domain of p65 NFκB protein and inhibits expression of a subset of NFκB target genes including PD-L1 polypeptides. This effect is mediated by the arginine-rich linker (R-linker) region in the N-terminal segment of RB polypeptides, but not the pocket domain, and CDK4/6 phosphorylation of the S249 and T252 residues in the R-linker largely enhances RB interaction with and inhibition of p65. As also described herein, a small bioactive S249/T252 phospho-mimicking polypeptide of an RB polypeptide was found to not only block irradiation-induced PD-L1 expression, but also to increase the anti-cancer efficacy of irradiation in vivo. These results demonstrate that the compositions provided herein can be used to reduce PD-L1 polypeptide expression in cancer cells within a mammal (e.g., a human) having cancer. These results also demonstrate that the compositions provided herein can be used in combination with other cancer treatment methods (e.g., radiation) and/or cancer treatment agents (e.g., chemotherapeutic agents) to increase cancer treatment effectiveness as compared to treatments not involving such a combination. Having the ability to reduce PD-L1 polypeptide expression in cancer cells as described herein and/or to improve cancer treatment effectiveness as described herein can allow patients and clinicians to improve patient treatment and improve patient outcomes.

In general, one aspect of this document features a method for treating a mammal having cancer. The method comprises, or consists essentially of, administering a polypeptide comprising an amino acid sequence as set forth in SEQ ID NO:1 to the mammal, wherein the level of PD-L1 expression of the cancer is reduced. The mammal can be a human. The cancer can be prostate cancer. The polypeptide can comprise an amino acid sequence as set forth in SEQ ID NO:2. The polypeptide can comprise an amino acid sequence as set forth in SEQ ID NO:3. The polypeptide can comprise an amino acid sequence as set forth in SEQ ID NO:4. The can comprise polypeptide can comprise an amino acid sequence as set forth in SEQ ID NO:5. The polypeptide can be less than 75 amino acid residues in length. The polypeptide can be less than 50 amino acid residues in length. The polypeptide can be less than 25 amino acid residues in length. The polypeptide can comprise an amino acid sequence as set forth in any one of SEQ ID NOs:6-179. The polypeptide can comprise an amino acid sequence as set forth in SEQ ID NO:6. The polypeptide can comprise an amino acid sequence as set forth in SEQ ID NO:7. The polypeptide can comprise an amino acid sequence as set forth in SEQ ID NO:8. The polypeptide can comprise an amino acid sequence as set forth in SEQ ID NO:9. The polypeptide can comprise an amino acid sequence as set forth in SEQ ID NO:10. The polypeptide can comprise an amino acid sequence as set forth in SEQ ID NO:11. The polypeptide can comprise an amino acid sequence as set forth in SEQ ID NO:12. The polypeptide can comprise an amino acid sequence as set forth in SEQ ID NO:13. The polypeptide can be administered to the mammal as the sole active ingredient. The level of PD-L1 expression of the cancer can be reduced by at least about 5 percent. The level of PD-L1 expression of the cancer can be reduced by at least about 10 percent. The level of PD-L1 expression of the cancer can be reduced by at least about 25 percent. The level of PD-L1 expression of the cancer can be reduced by at least about 50 percent. The level of PD-L1 expression of the cancer can be reduced to a level not detectable on cancer cells present within the mammal. The number of cancer cells present within the mammal can be reduced. The number of cancer cells present within the mammal can be reduced by at least about 10 percent. The number of cancer cells present within the mammal can be reduced by at least about 25 percent. The number of cancer cells present within the mammal can be reduced by at least about 50 percent. The method can further comprise administering radiation to the mammal. The number of cancer cells within the mammal can be reduced as compared to the number of cancer cells present in a comparable mammal having cancer administered the radiation and not administered the polypeptide. The cancer-free survival of the mammal can be increased as compared to the cancer-free survival of a comparable mammal having cancer administered the radiation and not administered the polypeptide. The method can further comprise administering a chemotherapeutic agent to the mammal. The chemotherapeutic agent can be camptothecin, taxane, a kinase inhibitor, gemcitabine, or a combination thereof. The number of cancer cells within the mammal can be reduced as compared to the number of cancer cells present in a comparable mammal having cancer administered the chemotherapeutic agent and not administered the polypeptide. The cancer-free survival of the mammal can be increased as compared to the cancer-free survival of a comparable mammal having cancer administered the chemotherapeutic agent and not administered the polypeptide.

In another aspect, this document features a polypeptide comprising, or consisting essentially of, an amino acid sequence as set forth in SEQ ID NO:1. The polypeptide can comprise an amino acid sequence as set forth in SEQ ID NO:2. The polypeptide can comprise an amino acid sequence as set forth in SEQ ID NO:3. The polypeptide can comprise an amino acid sequence as set forth in SEQ ID NO:4. The polypeptide can comprise an amino acid sequence as set forth in SEQ ID NO:5. The polypeptide can be less than 75 amino acid residues in length. The polypeptide can be less than 50 amino acid residues in length. The polypeptide can be less than 25 amino acid residues in length. The polypeptide can comprise an amino acid sequence as set forth in any one of SEQ ID NOs:6-179. The polypeptide can comprise an amino acid sequence as set forth in SEQ ID NO:6. The polypeptide can comprise an amino acid sequence as set forth in SEQ ID NO:7. The polypeptide can comprise an amino acid sequence as set forth in SEQ ID NO:8. The polypeptide can comprise an amino acid sequence as set forth in SEQ ID NO:9. The polypeptide can comprise an amino acid sequence as set forth in SEQ ID NO:10. The polypeptide can comprise an amino acid sequence as set forth in SEQ ID NO:11. The polypeptide can comprise an amino acid sequence as set forth in SEQ ID NO:12. The polypeptide can comprise an amino acid sequence as set forth in SEQ ID NO:13.

In another aspect, this document features a composition comprising a polypeptide, wherein the polypeptide comprises, or consists essentially of, an amino acid sequence as set forth in SEQ ID NO:1. The polypeptide can comprise an amino acid sequence as set forth in SEQ ID NO:2. The polypeptide can comprise an amino acid sequence as set forth in SEQ ID NO:3. The polypeptide can comprise an amino acid sequence as set forth in SEQ ID NO:4. The polypeptide can comprise an amino acid sequence as set forth in SEQ ID NO:5. The polypeptide can be less than 75 amino acid residues in length. The polypeptide can be less than 50 amino acid residues in length. The polypeptide can be less than 25 amino acid residues in length. The polypeptide can comprise an amino acid sequence as set forth in any one of SEQ ID NOs:6-179. The polypeptide can comprise an amino acid sequence as set forth in SEQ ID NO:6. The polypeptide can comprise an amino acid sequence as set forth in SEQ ID NO:7. The polypeptide can comprise an amino acid sequence as set forth in SEQ ID NO:8. The polypeptide can comprise an amino acid sequence as set forth in SEQ ID NO:9. The polypeptide can comprise an amino acid sequence as set forth in SEQ ID NO:10. The polypeptide can comprise an amino acid sequence as set forth in SEQ ID NO:11. The polypeptide can comprise an amino acid sequence as set forth in SEQ ID NO:12. The polypeptide can comprise an amino acid sequence as set forth in SEQ ID NO:13. The polypeptide can be the sole active ingredient of the composition.

In another aspect, this document features a nucleic acid molecule comprising a nucleic acid sequence encoding a polypeptide, wherein the polypeptide comprises, or consists essentially of, an amino acid sequence as set forth in SEQ ID NO:1. The polypeptide can comprise an amino acid sequence as set forth in SEQ ID NO:2. The polypeptide can comprise an amino acid sequence as set forth in SEQ ID NO:3. The polypeptide can comprise an amino acid sequence as set forth in SEQ ID NO:4. The polypeptide can comprise an amino acid sequence as set forth in SEQ ID NO:5. The polypeptide can be less than 75 amino acid residues in length. The polypeptide can be less than 50 amino acid residues in length. The polypeptide can be less than 25 amino acid residues in length. The polypeptide can comprise an amino acid sequence as set forth in any one of SEQ ID NOs:6-179. The polypeptide can comprise an amino acid sequence as set forth in SEQ ID NO:6. The polypeptide can comprise an amino acid sequence as set forth in SEQ ID NO:7. The polypeptide can comprise an amino acid sequence as set forth in SEQ ID NO:8. The polypeptide can comprise an amino acid sequence as set forth in SEQ ID NO:9. The polypeptide can comprise an amino acid sequence as set forth in SEQ ID NO:10. The polypeptide can comprise an amino acid sequence as set forth in SEQ ID NO:11. The polypeptide can comprise an amino acid sequence as set forth in SEQ ID NO:12. The polypeptide can comprise an amino acid sequence as set forth in SEQ ID NO:13. The molecule can be an expression vector. The expression vector can be a plasmid. The molecule can be a viral vector. The viral vector can be a pTsin lentiviral vector.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-K. RB suppresses PD-L1 mRNA expression. (A) PC-3 cell were treated with indicated chemicals for 24 hours. Cells were harvested for RT-qPCR. All data were shown as mean values±SD (n=3). * P<0.05, ** P<0.01, *** P<0.001. (B) PC-3 cells were infected with lentivirus expressing control or CDK4 and CDK6-specific shRNAS. 72 hours after infection, cells were harvested for RT-qPCR. All data were shown as mean values±SD (n=3). *** P<0.001. (C-F) PC-3 cells were infected with lentivirus expressing control or RB1-specific shRNAs. 48 hours after infection, PD-L1 protein levels were determined by western blotting (C), RT-qPCR (D), FACS (E), and IFC (F). All data were shown as mean values±SD (n=3). * P<0.05, ** P<0.01, *** P<0.001. (G) PD-L1 mRNA levels were determined by RT-qPCR in SKO and DKO mouse prostate tumor cells. All data were shown as mean values±SD (n=3). * P<0.05, ** P<0.01, *** P<0.001. (H-J) SKO (H), DKO (I), and Rb^{-/-}PrE (J) cells were treated with palbociclib for 24 hours. Cells were harvested for RT-qPCR. All data were shown as mean values±SD (n=3). * P<0.05, ** P<0.01, *** P<0.001. (K) The putative copy-number calls for RB1 were determined using GISTC 2.0, and the mRNA expression for CD274(PD-L1) was measured by log 2(RPKM), here RPKM stands for Reads Per Kilobase of transcript per Million mapped reads. All these data were obtained from cBioPortal on the SU2C prostate cancer dataset. The nonparametric Mann-Whitney U test was used for the statistical test.

FIGS. 2A-H. RB phosphorylation by CDK4 enhances RB interaction with NFκB protein p65. (A) Western blot analysis of reciprocal co-immunoprecipitation of endogenous RB1 and p65 proteins in PC-3 cells. (B) Schematic diagram depicting a set of p65 recombinant protein constructs. Western blot analysis of RB proteins in PC-3 cells whole cell lysate pulled down by GST or GST-p65 recombinant proteins. Stars indicated expected molecular weight. (C) Western blot analysis of RB proteins in PC-3 whole cell lysate pulled down by GST or GST-p65 recombinant proteins. Cell lysates were treated with or without λ, phosphatase before GST-pull down. (D) Western blot analysis of PC-3 whole cell lysates (WCL) and co-IP samples. Cell lysates were treated with or without λ, phosphatase before IP. (E) Western blot analysis of WCL and co-IP samples in DU145 cells 24 hours after transfected with indicated plasmids. (F) Western blot analysis of WCL and co-IP samples in PC-3 cells. Cells were treated with or without palbociclib (5 μM) for 24 hours before IP. (G) Western blot analysis of WCL and co-IP samples in PC-3 cells 48 hours after infected with the indicated shRNA. (H) Schematic diagram depicting a set of RB recombinant protein constructs. Western blot analysis of p65 proteins in PC-3 cells whole cell lysate pulled down by GST or GST-RB recombinant proteins. Stars indicate expected molecular weight.

FIGS. 3A-E. S249/T252 phosphorylation of RB and 161FQVTV165 motif (SEQ ID NO: 258) in p65 are required for p65-RB interaction. (A) Schematic diagram depicting CDK4/Cyclin D phosphorylation site in RB-N recombinant protein constructs. Western blot analysis of p65 proteins in PC-3 cells whole cell lysate pulled down by GST or GST-RB recombinant proteins. Stars indicated expected molecular weight. (B) Western blot analysis of in vitro transcribed and translated p65 proteins pulled down by GST or GST-RB recombinant proteins. Stars indicated expected molecular weight. (C) Schematic diagram depicting an evolutionally conserved 161FQVTV165 (SEQ ID NO: 258) (FxxxV)-centered basic (positive charge) motif in the RB-binding region in p65 and FxxxV-centered acidic (negative charge) motif in E1A-like inhibitor of differentiation-1 (EID1). FIG. 3C discloses SEQ ID NOS 258-264, respectively, in order of appearance. (D) PC-3 cells were transfected with indicated plasmids. Western blot analysis of HA-RB proteins in PC-3 whole cell lysate pulled down by GST or GST-p65 recombinant proteins. (E) Schematic diagram depicting a working model wherein introduction of negative charge by S249/T252 phosphorylation allows otherwise fully positively-charged RB-N to bind to positive charged 161FQVTV165 (SEQ ID NO: 258) motif in p65, a mechanism of action opposite to that between E1D1 and RB-N, thereby providing a mechanistic explanation for the observation that RB-p65 interaction was largely diminished by CDK4/6 inhibition. FIG. 3E discloses SEQ ID NOS 265-267, respectively, in order of appearance.

FIGS. 4A-G. RB globally regulates NFkB transcriptional program in cells. (A) Venn diagram indicating overlap up-regulated genes between palbociclib treatment versus DMSO and knockdown RB1 versus control identified by RNA-sequencing. (B) Ingenuity pathway analysis (IPA) of 897 genes commonly upregulated by palbociclib treatment and RB knockdown in PC-3 cell, ranked by P-value. (C) Heatmap showing commonly upregulated genes by palbociclib treatment and RB knockdown, highlighting a subset of genes involved in T cell regulation pathway and TNFα signaling via NF-κB. (D) Gene Set Enrichment Analysis (GSEA) for 897 genes commonly upregulated by palbociclib treatment and RB knockdown. (E) RNA-seq track views from UCSC Genome Browser four genes (PD-L1, GADD45B, NR4A2, and CD83) commonly upregulated by palbociclib treatment and RB knockdown. (F, G) PC-3 cell were treated with or without palbociclib (5 μM) for 24 hours or PC-3 cells were infected with lentivirus expressing control shRNA or RB-specific shRNA following puromycin selection after 48 hours infection. Cells were harvested for RT-qPCR (F) and ChIP-qPCR (G) analysis of p65 binding at the promotor of PD-L1, GADD45B, NR4A2, and CD83. All data were shown as mean values±SD (n=3). * P<0.05, ** P<0.01, ***P<0.001.

FIGS. 5A-K. The importance of S249/T252 phosphorylation of RB in regulation of PD-L1 expression. (A, B) PC-3 cells were infected with lentivirus expressing control shRNA, RB1-specific, p65-specific, or both shRNA. 48 hours post puromycin selection, cells were harvested for western blotting (A) and RT-qPCR analysis (B). All data were shown as mean values±SD (n=3). * P<0.05, ** P<0.01, *** P<0.001. (C, D) DU145 cells were transfected with indicated plasmids. 24 hours after transfection, cells were harvested for western blotting analysis (C) and RT-qPCR analysis (D). All data were shown as mean values±SD (n=3). * P<0.05, ** P<0.01, *** P<0.001. (E, F) DU145 cells were transfected with indicated plasmids. 24 hours after transfection, cells were harvested for western blotting analysis (E) and RT-qPCR analysis (F). All data were shown as mean values±SD (n=3). * P<0.05, ** P<0.01, *** P<0.001. (G, H) PC-3 cells were infected with lentivirus expressing control shRNA or p65-specific shRNAs. After 24 hours, cells were further transfected with empty vector or SFB-RL-S249D/T252D for another 24 hours followed by western blot analysis (G) and RT-qPCR analysis (H). All data were shown as mean values±SD (n=3). * P<0.05, ** P<0.01, *** P<0.001. (I) Representative images of IHC of anti-PD-L1 and anti-pRB-S249/T252 antibodies of TMA (n=145 TMA elements) tissue sections. Scale bars were shown as indicated. (J) Heatmap showing IHC score of PD-L1 and pRB-S249/T252 in TMA. (K) Correlation analysis of IHC score for expression of PD-L1 and pRB-S249/T252 polypeptides in prostate cancer patient specimens (n=145 TMA elements). Pearson's product-moment correlation co-efficiency and the P values were also shown.

FIGS. 6A-J. The small S249/T252 phosphorylation-mimicking polypeptide of RB blocks irradiation-induced PD-L1 expression and inhibits cancer immune evasion. (A, B) PC-3 cells were infected with lentivirus expressing control shRNA or p65-specific or shRNA. 24 hours after puromycin selection, cells were treated with or without gamma irradiation (IR) (12 Gy). 48 hours after IR treatment, cells were harvested for western blotting analysis (A) and RT-qPCR analysis (B). All data were shown as mean values±SD (n=3). * P<0.05, ** P<0.01, *** P<0.001. (C, D) PC-3 cells were treated with without gamma irradiation (12 Gy). Cells were harvested at the indicated time point for western blotting analysis (C) and RT-qPCR analysis (D). All data were shown as mean values±SD (n=3). * P<0.05, ** P<0.01, *** P<0.001. (E) Western blot analysis of WCL and co-IP samples in PC-3 cells 48 hours after treated with or without gamma irradiation (12 Gy). (F) ChIP-qPCR analysis of p65 binds at the PD-L1 promotor in PC-3 cells 48 hours after treated with or without gamma irradiation (12 Gy). All data were shown as mean values±SD (n=3). * P<0.05, ** P<0.01, *** P<0.001. (G, H) PC-3 cells were treated with or without gamma irradiation (12 Gy). 24 hours post treatment, cells were transfected with indicated constructs. 24 hours after transfection, cells were harvested for western blotting analysis (G) and RT-qPCR analysis (H). All data were shown as mean values±SD (n=3). * P<0.05, ** P<0.01, *** P<0.001. (I) PTEN-CaP8 mouse prostate cancer cells were transduced with lentivirus for control vector or the small RB S249/T252-phosphorylation-mimicking polypeptide RL-S249D/T252D. 72 hours after purimycin selection, 5×10⁶ control or RL-S249D/T252D-expressing cells were injected subcutaneously into C57BL/6 mice. Mice (n=8/group) were treated with gamma irradiation (12 Gy) or mock treated, and with anti-PD-L1 (200 μg) or non-specific IgG for 45 days. (J) The absolute number of CD45⁺CD8⁺ T cells, CD45⁺CD4⁺ T cells, and CD11b⁺Gr1⁺ myeloid cells of implanted PTEN-CaP8 in tumors treated with indicated agents was analyzed by FACS. All data were shown as mean values±SD (n=8). ns, not significant, *** P<0.001.

FIG. 7 is a schematic diagram of the indicated pathways.

FIGS. 8A-D. (A) PC-3, MIA PaCa-2, H1299, SK-Hep1, and PTEN-CaP8 cells were treated with different doses of palbociclib for 24 hours. Cells were harvested for RT-qPCR. All data were shown as mean values±SD (n=3). * P<0.05, ** P<0.01. (B) PC-3 cell were treated with indicated chemicals for 24 hours. Cells were harvested for RT-qPCR. All data were shown as mean values±SD (n=3). Ns, not significant, * P<0.05, ** P<0.01. (C, D) PC-3 cells were infected with lentivirus expressing control shRNA or RB1-specific shRNA. 24 hours after puromycin selection, cells were treated with or without Helenalin (2 μM) for 24 hours. Cells were harvested for western blotting analysis (C) and RT-qPCR (D). All data were shown as mean values±SD (n=3). Ns, not significant, *** P<0.001.

FIGS. 9A-D. (A) Western blot analysis of WCL and co-IP samples in PC-3 cells 48 hours after infected with indicated shRNAs. (B) Western blot analysis of WCL and co-IP samples in PC-3 cells 48 hours after transfected with indicated constructs. (C) Schematic diagram depicting the domain structure of RB, p107, and p130 of the pocket protein family. (D) Amino acid sequence alignment of the N-terminal of RB, p107 (RBL1), and p130 (RBL2). FIG. 9D discloses SEQ ID NOS 268-270, respectively, in order of appearance.

FIGS. 10A-G. (A) Schematic diagram depicting a set of RB-N recombinant protein constructs. Western blot analysis of p65 proteins in PC-3 cells whole cell lysate pulled down by GST or GST-RB-N recombinant proteins. Stars indicate expected molecular weight. (B) Western blot analysis of in vitro transcribed and translated p65 proteins pulled down by GST or GST-RB recombinant proteins. Stars indicate expected molecular weight. (C) Western blot analysis of WCL and co-IP samples in 293T cells 48 h after transfected with indicated constructs. (D) Schematic diagram depicting an evolutionally conserved R-linker region in the N-terminal of RB. FIG. 10D discloses SEQ ID NOS 265 and 271-274, respectively, in order of appearance. (E) Western blot analysis of WCL and co-IP samples in 293T cells 48 hours after transfected with indicated constructs. (F) PC-3 cells were transfected with indicated plasmids. Western blot analysis of HA-RB proteins in PC-3 whole cell lysate pulled down by GST or GST-p65 recombinant proteins. Stars indicate expected molecular weight. (G) Schematic diagram depicting fully positively charged RB-N allows otherwise negatively charged by S249/T252 phosphorylation to bind to negatively charged 166FSLMV170 motif (SEQ ID NO: 286) in EID1. FIG. 10G discloses SEQ ID NOS 265-266 and 275, respectively, in order of appearance.

FIGS. 11A-H. (A) Heatmap showing differentially expressed genes (FDR<0.01) between palbociclib treatment versus DMSO and knockdown RB1 versus control. Gene expression values (FPKM) were Z-score normalized. FPKM, Fragments Per Kilobase of transcript per Million mapped reads. (B) UCSC Genome Browser screenshots of meta-analysis of published RB ChIP high throughput sequencing (ChIP-seq) data showing RB binding to CD274 (PD-L1) gene locus in K562 cells. PD-L1-prom oligonucleotide DNA containing NF-κB binding sequence was shown in the RB binding peak in the PD-L1 gene, the corresponding mutant PD-L1-prom oligonucleotide DNA was shown below. FIG. 11B discloses SEQ ID NOS 279-280, respectively, in order of appearance. (C) ChIP-qPCR analysis of RB binds at the PD-L1 promotor in PC-3 cells. All data were shown as mean values±SD (n=3). *** P<0.001. (D) ChIP-reChIP analysis examining the co-localization of p65 and RB in the PD-L1 promoters. All data were shown as mean values±SD (n=3). ** P<0.01. (E) UCSC Genome Browser screenshots of meta-analysis of published RB ChIP high throughput sequencing (ChIP-seq) data showing RB binding to GADD45B, NR4A2, and CD83 gene locus containing NF-κB binding sequence in K562 cells. FIG. 11E discloses SEQ ID NOS 276-278, respectively, in order of appearance. (F) ChIP-qPCR analysis of RB binds at the GADD45B, NR4A2, and CD83 promotor in PC-3 cells. All data were shown as mean values±SD (n=3). * P<0.05, ** P<0.01. (G) ChIP-reChIP analysis examining the co-localization of p65 and RB in the GADD45B, NR4A2, and CD83 promoters. All data were shown as mean values±SD (n=3). ** P<0.01. (H) PC-3 cells were treated with or without palbociclib (5 μM) for 24 hours, or PC-3 cell were infected with lentivirus expressing control shRNA or RB-specific shRNA following puromycin selection after 48 hours infection. Nuclear extracts were subjected to EMSA with the biotin-labeled oligonucleotide NF-κB site of the PD-L1 promoter in the absence and presence of the indicated unlabeled or unlabeled-mutated competitors.

FIGS. 12A-C. (A, B) PANC-1, H1299, and SK-Hep1 were infected with lentivirus expressing control or RB-specific shRNAs following puromycin selection. 48 hours post infection, cells were transfected with indicated construct. 24 hours after transfection, cells were harvested for western blotting (A) and RT-qPCR (B). All data were shown as mean values±SD (n=3). “ns”=not significant, *** P<0.001. (C) Western blot analysis of expression of PD-L1, RB, pRB-S249/T252, pRB-5795, and ERK2 in prostate cancer cell lines indicated.

FIGS. 13A-F. (A, B) PC-3 cells were treated with or without gamma irradiation (IR) (12 Gy). 24 hours post treatment, cells were treated with or without Helenalin (2 μM). 24 hours after treatment, cells were harvested for western blotting analysis (A) and RT-qPCR analysis (B). All data were shown as mean values±SD (n=3). “ns”=not significant, *** P<0.001. (C) PTEN-CaP8 cells were infected with lentivirus expression Tsin control or Tsin-RL S249D/T252D peptide. 72 hours post infection, cell were harvested for western blotting analysis. (D, E) PTEN-CaP8 cells were infected with lentivirus expression Tsin control or Tsin-RL S249D/T252D peptide. 72 hours post infection, cells were treated with or without gamma irradiation (12 Gy) for 48 hours. Cells were harvested for MTS assay (D) and RT-qPCR (E). All data were shown as mean values±SD (n=3). “ns”=not significant, ** P<0.01; *** P<0.001. (F) Schematic diagram depicting the treatment plan for mice bearing subcutaneous PTEN-CaP8 tumors.

FIGS. 14A-G. RB functions as a negative regulator of NFκB signaling. (A) PC-3 infected with lentivirus for indicated shRNAs were treated with different chemicals and cells viability was measured by MTS assay. Heat map showing the normalized IC₅₀ ratio (log 2(IC₅₀ratio)). (B-D) PC-3 cells infected with lentivirus expressing indicated shRNAs were harvested for western blots (B), FACS (C), and RT-qPCR (D). All data were shown as mean values±SD (n=3). ns, not significant, * P<0.05, ** P<0.01, *** P<0.001. (E, F) PC-3 cells infected with indicated lentivirus were injected s.c. into NSG mice, and tumors were harvested and photographed at day 21 (E). Data in (F) were shown as means±SD (n=5). ns, not significant, * P<0.05. (G) A model depicting RB functions as a negative regulator of NFκB (p65/p50), providing an explanation of the finding that depletion of RB offsets the death of PTEN-null cells induced by either MAP3K7 or CHD1 deletion.

FIG. 15. The top panel contains GST-tagged mammalian expression vector sequences for the N-terminal of wild-type (WT) p65 and alanine (A) and arginine (R) mutants. The bottom panel contains results where PC-3 cells were transfected with the indicated plasmids followed by western blot analysis of PC-3 WCL pulled down by GST or GST-p65 recombinant proteins. FIG. 15 discloses SEQ ID NOS 281-283, respectively, in order of appearance.

FIGS. 16A-K. Mutual exclusivity of PTEN and MAP3K7 gene deletion in human cancers and CDK4/6 inhibition-mediated blockage of MAP3K7 deficiency-induced death of PTEN-null cells. (A) Genetic alterations of MAP3K7 and PTEN genes in the TCGA prostate cancer dataset. (B) Genetic alterations of MAP3K7 and PTEN genes in lymphoid neoplasm diffuse large B-cell lymphoma, stomach adenocarcinoma, pancreatic cancer, liver cancer, bladder urothelial carcinoma, and lung adenocarcinoma. (C-E) PC-3 cells were infected with lentivirus expressing control or MAP3K7-specific shRNAs. 72 hours after infection, cells were harvested for western blotting (C), cell growth assay (D), and colony formation assay (E). All data were shown as mean values±SD (n=3). ns, not significant, * P<0.05 comparing to the shControl group. (F-H) LNCaP-RF prostate cancer cells were infected with lentivirus expressing control or MAP3K7-specific shRNAs. 72 hours after infection, cells were harvested for western blotting (F), cell growth assay (G), and colony formation assay (H). All data are shown as mean values±SD (n=3). ns, not significant, * P<0.05 comparing to the shControl group. (I-K) PC-3 cells were infected with lentivirus expressing control, MAP3K7 or CHD1-specific shRNAs. 72 hours after infection, cells were treated with DMSO or palbociclib (5 μM). 24 hours post treatment, cells were harvested for western blotting (I), FACS (J), and RT-qPCR (K). For (J), all data were shown as mean values±SD (n=3). ns, not significant, * P<0.05, *** P<0.001. For (K), all data were shown as mean values±SD (n=3). ns, not significant, * P<0.05, *** P<0.001 comparing to the shControl group.

FIGS. 17A-I. Negative regulation of MAP3K7-IKK-NFκB and CHD1-NFκB signaling by RB. (A-C) PC-3 cells were infected with lentivirus expressing indicated constructs. 72 hours after infection, cells were harvested for western blots (A), MTS assay (B) and RT-qPCR analysis of expression of NFκB target genes (Red) and E2F1 target genes (Blue) (C). For (B), all data were shown as mean values±SD (n=6). ns, not significant, *** P<0.001. For (C), all data were shown as mean values±SD (n=3). ns, not significant, *** P<0.001. (D, E) PC-3 cells were infected with lentivirus expressing indicated constructs. 72 hours after infection and puromycin selection, cells were injected s.c. into the right flank of NSG mice, and tumor growth was measured for 21 days. Tumors in each group at day 21 were harvested and photographed (D). Data in (E) were shown as means±SD (n=5). ns, not significant, *** P<0.001. (F) Genetic alterations of MAP3K7 and RB1 genes in the TCGA prostate cancer dataset. Co-occurrence of MAP3K7 and RB1 alterations was statistically significant (P<0.001). (G) Genetic alterations of CHD1 and RB1 genes in the TCGA prostate cancer dataset. Co-occurrence of CHD1 and RB1 alterations was statistically significant (P<0.001). (H) PC-3 cells were infected with lentivirus expressing indicated shRNAs. 72 hours after infection, cells were harvested for western blots and RT-qPCR analysis of expression of NFκB target genes. All data were shown as mean values±SD (n=3). ns, not significant, * P<0.05, ** P<0.01, *** P<0.001. (I) PC-3 cells were treated with DMSO, palbociclib (5 μM), IKK-16 (5 μM) or ACHP (20 μM). 24 hours post treatment, cells were harvested for western blotting and RT-qPCR. All data were shown as mean values±SD (n=3). ns, not significant, * P<0.05, ** P<0.01, *** P<0.001.

FIGS. 18A-G. p65 binds to RB in cell lines of different cancer types and their interaction occurs primarily in the nucleus. (A) Western blot analysis of co-immunoprecipitated endogenous p50, p65 and RB proteins in PC-3 cells. (B) Western blot analysis of whole cell lysate (WCL), cytosolic fractionation and nuclear fractionation (Input) and reciprocally co-immunoprecipitated endogenous RB and p65 proteins in PC-3 cells. (C) Western blot analysis of whole cell lysate (WCL) and co-IP samples from PC-3 cells 24 hours after treated with or without TNFα (20 ng/mL). (D) Western blot analysis of whole cell lysate (WCL) and co-IP samples from PC-3 cells 24 hours after treated with or without TNFα (20 ng/mL). Western blot bands of co-IPed RB protein were quantified by ImageJ software and normalized to the quantified value of IP-ed RB in cells without TNFα treatment. The normalized values were further normalized to the value in cells infected with shControl. (E, F) PC-3 cell lysates (E) or primary SKO (Rb+/+) cell lyates (F) were undepleted (preimmune sample) or immuno-depleted with anti-RB (left)- or anti-IκB-α antibody (right)-bound beads for five times, and supernatants and IP products were immunoblotted with indicated antibodies. Western blot bands of p65 protein were quantified by ImageJ software and normalized to the quantified value of p65 protein in cell lysate without immuno-depletion (preimmune sample). (G) Western blot analysis of reciprocally co-immunoprecipitated endogenous RB and p65 proteins in PANC-1, H1299 and SK-Hep1 cell lines.

FIGS. 19A-G. The involvement of S249/T252 phosphorylation of RB-N in its interaction with p65. (A-C) Western blot analysis of whole cell lysate (WCL) and co-IP samples of PANC-1 (A), H1299 (B), and SK-Hep1 (C) cell lines 48 hours after transfected with indicated constructs. (D) Western blot analysis of whole cell lysate (WCL) and co-IP samples of PC-3 cells after transfected with indicated constructs and cultured in serum-free medium for 24 hours followed by treatment with or without 100 mM NaCl for 4 hours. (E) PC-3 cells were cultured in serum-free medium for 24 hours followed by treatment with or without 100 mM NaCl for 4 hours. Cell lysates were then undepleted (preimmune sample) or immuno-depleted with anti-E2F1 (left)- or anti-p65 antibody (right)-bound beads for five times, and supernatants and IP products were immunoblotted with indicated antibodies. Western blot bands of RB protein were quantified by ImageJ software and normalized to the quantified value of p65 protein in cell lysate without immuno-depletion (preimmune sample). (F) Western blot analysis of whole cell lysate (WCL) and co-IP samples of 293T cells after transfected with indicated constructs and cultured in serum-free medium for 24 hours followed by treatment with or without 100 mM NaCl for 4 hours. (G) Top, GST-tagged mammalian expression vectors for the N-terminal of wild-type (WT) EID1 and alanine (A) and arginine (R) mutants. Bottom, PC-3 cells were transfected with indicated plasmids followed by western blot analysis of PC-3 WCL pulled down by GST or GST-EID1 recombinant proteins. Asterisks indicate proteins at the expected molecular weight. FIG. 19G discloses SEQ ID NOS 287 and 284-285, respectively, in order of appearance.

FIG. 20. Effect of RB depletion and CDK4/6 inhibition on p65 binding in the promoter of PD-L1 gene. PC-3 cells were infected with lentivirus expressing control or RB-specific shRNAs followed by puromycin selection for 48 hours, treated with or without TNFα (20 ng/mL) for 24 hours. Nuclear extracts were isolated for EMSA with the biotin-labeled oligonucleotide (PD-L1-prom) in the absence or presence of the indicated antibody. DPC, DNA-protein complex.

FIGS. 21A-N. Effect of RB depletion, cell cycle, and RB-N S249/T252 phospho-mimicking peptide on PD-L1 expression in cell lines of various cancer types. (A-C) LNCaP cells were infected with lentivirus expressing control or RB1-specific shRNAs. 48 hours after infection, PD-L1 expression was determined by western blotting (A), RT-qPCR (B), and FACS (C). All data were shown as mean values±SD (n=3). ** P<0.01. (D) Pd-l1 protein and mRNA levels were determined by western blotting in primary SKO and DKO mouse prostate tumor cell lines. All data were shown as mean values±SD (n=3). *** P<0.001. (E-I) PC-3 cells were synchronized in M phase by nocodazole and release back into the cell cycle. At the different time points, cells were harvested for FACS analysis for monitoring cell cycle (E), western blot analysis of expression of PD-L1, pRB-S249/T252, p-p65-5536, p65, CYCLIN B, CYCLIN D1, CYCLIN E and ERK2 (loading control) (F), RT-qPCR analysis of PD-L1 (G), TNFA (H) and CD83 (I) mRNA. All data were shown as mean values±SD (n=3). (J) PC-3 cells were synchronized in M phase by nocodazole or in G1/S phase by double thymidine. RT-qPCR analysis of PD-L1 mRNA after the concomitant addition of actinomycin D (10 μg/mL). All data were shown as mean values±SD (n=3). ns, not significant, two-way ANOVA. (K, L) PC-3 cells were transfected with mammalian expression vector for SFB-tagged EV (empty vector) or RL S249D/T252D mutant peptide. 24 hours after transfection. Cells were treated with or without TNFα (20 ng/mL). 24 hours post treatment, cells were harvested for western blot analysis (K) and RT-qPCR analysis of expression of NFκB target genes (L). All data were shown as mean values±SD (n=3). ns, not significant, * P<0.05, ** P<0.01, *** P<0.001. (M) PC-3 cells were transfected with mammalian expression vector for SFB-tagged EV (empty vector) or RL S249D/T252D mutant peptide. 24 hours after transfection, cells were harvested for cytosolic and nuclear fractionation followed by western blot analysis with indicated antibodies. (N) PC-3 cells were treated with without gamma radiation (12 Gy). 24 hours post treatment, cells were transfected with mammalian expression vector for SFB-tagged EV or RL S249D/T252D mutant peptide followed by TNFα (20 ng/mL) treatment for 24 hours. Nuclear extracts were isolated for EMSA with the biotin-labeled oligonucleotide. DPC, DNA-protein complex.

FIGS. 22A-B. Effect of expression of RB-N S249/T252 phospho-mimicking peptide on PD-L1 expression in cells treated with radiation. (A, B) PC-3 cells were treated with without gamma radiation (12 Gy). Cells were harvested at the indicated time points for western blotting (A) and RT-qPCR analysis (B).

### DETAILED DESCRIPTION

This document provides methods and materials for treating cancer. For example, this document provides polypeptides, isolated nucleic acids, vectors (e.g., viral vectors), host cells, compositions containing polypeptides, compositions containing vectors, methods for reducing PD-L1 expression within cancer cells, methods for treating cancer, and methods for increasing the effectiveness that a cancer treatment method and/or cancer agent exhibits against cancer within a mammal (e.g., a human).

In some cases, a polypeptide provided herein for reducing PD-L1 expression within cancer cells, for treating cancer, and/or for increasing the effectiveness of another cancer treatment method and/or cancer agent against cancer within a mammal as described herein can include the following amino acid sequence: NGX₁PRX₂PR, where X₁ is D or E, and X₂ is D or E (SEQ ID NO:1). For example, a composition provided herein that can be administered to a mammal to reduce PD-L1 expression within cancer cells and/or to treat cancer as described herein can include a polypeptide having NGDPRDPR (SEQ ID NO:2), NGDPREPR (SEQ ID NO:3), NGEPRDPR (SEQ ID NO:4), or NGEPREPR (SEQ ID NO:5). In some cases, a composition provided herein can include a polypeptide that is from about 8 to about 200 (e.g., from 8 to 180, from 8 to 170, from 8 to 160, from 8 to 150, from 8 to 140, from 8 to 130, from 8 to 120, from 8 to 110, from 8 to 100, from 8 to 90, from 8 to 80, from 8 to 70, from 8 to 60, from 8 to 50, from 8 to 40, from 8 to 30, from 8 to 20, or from 8 to 10) amino acid residues in length. For example, a composition provided herein can include a polypeptide having the amino acid sequence as set forth in any one of SEQ ID NOs:2-179 with the total amino acid length of the polypeptide being from about 8 to about 50 (e.g., from 8 to 45, from 8 to 40, from 8 to 35, from 8 to 30, from 8 to 25, from 8 to 24, from 8 to 23, from 8 to 22, from 8 to 21, from 8 to 20, from 8 to 19, from 8 to 18, from 8 to 17, from 8 to 16, from 8 to 15, from 8 to 14, from 8 to 13, from 8 to 12, from 8 to 11, from 8 to 10, from 8 to 9). Examples of polypeptides that can be used to treat cancer as described herein include, without limitation, polypeptides that include an amino acid sequence set forth in Table 1.

TABLE 1
Amino acid sequences.

| Amino Acid Sequence | SEQ ID NO: |
|---|---|
| NGDPRDPR | 2 |
| NGDPREPR | 3 |
| NGEPRDPR | 4 |
| NGEPREPR | 5 |
| PINGDPRDPRRGQNRSARIAKQL | 6 |
| PINGEPRDPRRGQNRSARIAKQL | 7 |
| PINGDPREPRRGQNRSARIAKQL | 8 |
| PINGEPREPRRGQNRSARIAKQL | 9 |
| PFNGDPRDPRRGQNRSARIAKQL | 10 |
| PFNGEPRDPRRGQNRSARIAKQL | 11 |
| PFNGDPREPRRGQNRSARIAKQL | 12 |
| PFNGEPREPRRGQNRSARIAKQL | 13 |
| NGSPRDPR | 14 |
| NGSPREPR | 15 |
| NGDPRTPR | 16 |
| NGEPRTPR | 17 |
| PINGSPRDPRRGQNRSARIAKQL | 18 |
| PINGSPREPRRGQNRSARIAKQL | 19 |
| PINGDPRTPRRGQNRSARIAKQL | 20 |
| PINGEPRTPRRGQNRSARIAKQL | 21 |
| PFNGSPRDPRRGQNRSARIAKQL | 22 |
| PFNGSPREPRRGQNRSARIAKQL | 23 |
| PFNGDPRTPRRGQNRSARIAKQL | 24 |
| PFNGEPRTPRRGQNRSARIAKQL | 25 |
| INGDPRDPRRGQNRSARIAKQL | 26 |
| INGEPRDPRRGQNRSARIAKQL | 27 |
| INGDPREPRRGQNRSARIAKQL | 28 |
| INGEPREPRRGQNRSARIAKQL | 29 |
| FNGDPRDPRRGQNRSARIAKQL | 30 |
| FNGEPRDPRRGQNRSARIAKQL | 31 |
| FNGDPREPRRGQNRSARIAKQL | 32 |
| FNGEPREPRRGQNRSARIAKQL | 33 |
| INGSPRDPRRGQNRSARIAKQL | 34 |
| INGSPREPRRGQNRSARIAKQL | 35 |
| INGDPRTPRRGQNRSARIAKQL | 36 |
| INGEPRTPRRGQNRSARIAKQL | 37 |
| FNGSPRDPRRGQNRSARIAKQL | 38 |
| FNGSPREPRRGQNRSARIAKQL | 39 |
| FNGDPRTPRRGQNRSARIAKQL | 40 |
| FNGEPRTPRRGQNRSARIAKQL | 41 |
| NGDPRDPRRGQNRSARIAKQL | 42 |
| NGEPRDPRRGQNRSARIAKQL | 43 |
| NGDPREPRRGQNRSARIAKQL | 44 |
| NGEPREPRRGQNRSARIAKQL | 45 |
| NGSPRDPRRGQNRSARIAKQL | 46 |
| NGSPREPRRGQNRSARIAKQL | 47 |
| NGDPRTPRRGQNRSARIAKQL | 48 |
| NGEPRTPRRGQNRSARIAKQL | 49 |
| NGDPRDPRRGQNRSARIAKQL | 50 |
| NGEPRDPRRGQNRSARIAKQL | 51 |
| NGDPREPRRGQNRSARIAKQL | 52 |
| NGEPREPRRGQNRSARIAKQL | 53 |
| NGSPRDPRRGQNRSARIAKQL | 54 |
| NGSPREPRRGQNRSARIAKQL | 55 |
| NGDPRTPRRGQNRSARIAKQL | 56 |
| NGEPRTPRRGQNRSARIAKQL | 57 |
| NGDPRDPRRG | 58 |
| NGEPRDPRRG | 59 |
| NGDPREPRRG | 60 |
| NGEPREPRRG | 61 |
| NGSPRDPRRG | 62 |
| NGSPREPRRG | 63 |
| NGDPRTPRRG | 64 |
| NGEPRTPRRG | 65 |
| NGDPRDPRRGQNRS | 66 |
| NGEPRDPRRGQNRS | 67 |
| NGDPREPRRGQNRS | 68 |
| NGEPREPRRGQNRS | 69 |
| NGSPRDPRRGQNRS | 70 |
| NGSPREPRRGQNRS | 71 |
| NGDPRTPRRGQNRS | 72 |
| NGEPRTPRRGQNRS | 73 |
| NGDPRDPRRGQNRSARI | 74 |
| NGEPRDPRRGQNRSARI | 75 |
| NGDPREPRRGQNRSARI | 76 |
| NGEPREPRRGQNRSARI | 77 |
| NGSPRDPRRGQNRSARI | 78 |
| NGSPREPRRGQNRSARI | 79 |
| NGDPRTPRRGQNRSARI | 80 |
| NGEPRTPRRGQNRSARI | 81 |
| NGDPRDPRRGQNRSARIAK | 82 |
| NGEPRDPRRGQNRSARIAK | 83 |
| NGDPREPRRGQNRSARIAK | 84 |
| NGEPREPRRGQNRSARIAK | 86 |
| NGSPRDPRRGQNRSARIAK | 87 |
| NGSPREPRRGQNRSARIAK | 88 |
| NGDPRTPRRGQNRSARIAK | 89 |
| NGEPRTPRRGQNRSARIAK | 90 |

In some cases, a polypeptide provided herein for reducing PD-L1 expression within cancer cells, for treating cancer, and/or for increasing the effectiveness of another cancer treatment method and/or cancer agent against cancer within a mammal as described herein can include the following amino acid sequence: NGX₁PRX₂PR, where X₁ is D or E, and X₂ is D or E (SEQ ID NO:1) with an N-terminal and/or C-terminal cell targeting sequence. An N-terminal and/or C-terminal cell targeting sequence such as eight D-arginine residues (SEQ ID NO: 91) can act as a signal peptide as described elsewhere (see, e.g., Jameson, Nature Medicine, 19:626-630 (2013)) to facilitate the entry of a polypeptide (e.g., an RB-phospho-mimicking polypeptide) provided herein into cells (e.g., cancer cells). Examples of cell targeting sequences that can be located at the N-terminus and/or C-terminus of a polypeptide provided herein (e.g., a polypeptide having an amino acid sequence set forth in Table 1) can be six, seven, eight, nine, ten, or more consecutive D-arginine residues (e.g., RRRRRRRR (SEQ ID NO:91)). Examples of such polypeptides are set forth in Table 2.

TABLE 2
Polypeptide containing cell targeting sequence.

| | SEQ |
|---|---|
| | ID |
| Amino Acid Sequence | NO: |
| RRRRRRRRNGDPRDPR | 92 |
| RRRRRRRRNGDPREPR | 93 |
| RRRRRRRRNGEPRDPR | 94 |
| RRRRRRRRNGEPREPR | 95 |
| RRRRRRRRPINGDPRDPRRGQNRSARIAKQL | 96 |
| RRRRRRRRPINGEPRDPRRGQNRSARIAKQL | 97 |
| RRRRRRRRPINGDPREPRRGQNRSARIAKQL | 98 |
| RRRRRRRRPINGEPREPRRGQNRSARIAKQL | 99 |
| RRRRRRRRPFNGDPRDPRRGQNRSARIAKQL | 100 |
| RRRRRRRRPFNGEPRDPRRGQNRSARIAKQL | 101 |
| RRRRRRRRPFNGDPREPRRGQNRSARIAKQL | 102 |
| RRRRRRRRPFNGEPREPRRGQNRSARIAKQL | 103 |
| RRRRRRRRNGSPRDPR | 104 |
| RRRRRRRRNGSPREPR | 105 |
| RRRRRRRRNGDPRTPR | 106 |
| RRRRRRRRNGEPRTPR | 107 |
| RRRRRRRRPINGSPRDPRRGQNRSARIAKQL | 108 |
| RRRRRRRRPINGSPREPRRGQNRSARIAKQL | 109 |
| RRRRRRRRPINGDPRTPRRGQNRSARIAKQL | 110 |
| RRRRRRRRPINGEPRTPRRGQNRSARIAKQL | 111 |
| RRRRRRRRPFNGSPRDPRRGQNRSARIAKQL | 112 |
| RRRRRRRRPFNGSPREPRRGQNRSARIAKQL | 113 |
| RRRRRRRRPFNGDPRTPRRGQNRSARIAKQL | 114 |
| RRRRRRRRPFNGEPRTPRRGQNRSARIAKQL | 115 |
| RRRRRRRRINGDPRDPRRGQNRSARIAKQL | 116 |
| RRRRRRRRINGEPRDPRRGQNRSARIAKQL | 117 |
| RRRRRRRRINGDPREPRRGQNRSARIAKQL | 118 |
| RRRRRRRRINGEPREPRRGQNRSARIAKQL | 119 |
| RRRRRRRRFNGDPRDPRRGQNRSARIAKQL | 120 |
| RRRRRRRRFNGEPRDPRRGQNRSARIAKQL | 121 |
| RRRRRRRRFNGDPREPRRGQNRSARIAKQL | 122 |
| RRRRRRRRFNGEPREPRRGQNRSARIAKQL | 123 |
| RRRRRRRRINGSPRDPRRGQNRSARIAKQL | 124 |
| RRRRRRRRINGSPREPRRGQNRSARIAKQL | 125 |
| RRRRRRRRINGDPRTPRRGQNRSARIAKQL | 126 |
| RRRRRRRRINGEPRTPRRGQNRSARIAKQL | 127 |
| RRRRRRRRFNGSPRDPRRGQNRSARIAKQL | 128 |
| RRRRRRRRFNGSPREPRRGQNRSARIAKQL | 129 |
| RRRRRRRRFNGDPRTPRRGQNRSARIAKQL | 130 |
| RRRRRRRRFNGEPRTPRRGQNRSARIAKQL | 131 |
| RRRRRRRRNGDPRDPRRGQNRSARIAKQL | 132 |
| RRRRRRRRNGEPRDPRRGQNRSARIAKQL | 133 |
| RRRRRRRRNGDPREPRRGQNRSARIAKQL | 134 |
| RRRRRRRRNGEPREPRRGQNRSARIAKQL | 135 |
| RRRRRRRRNGSPRDPRRGQNRSARIAKQL | 136 |
| RRRRRRRRNGSPREPRRGQNRSARIAKQL | 137 |
| RRRRRRRRNGDPRTPRRGQNRSARIAKQL | 138 |
| RRRRRRRRNGEPRTPRRGQNRSARIAKQL | 139 |
| RRRRRRRRNGDPRDPRRGQNRSARIAKQL | 140 |
| RRRRRRRRNGEPRDPRRGQNRSARIAKQL | 141 |
| RRRRRRRRNGDPREPRRGQNRSARIAKQL | 142 |
| RRRRRRRRNGEPREPRRGQNRSARIAKQL | 143 |
| RRRRRRRRNGSPRDPRRGQNRSARIAKQL | 144 |
| RRRRRRRRNGSPREPRRGQNRSARIAKQL | 145 |
| RRRRRRRRNGDPRTPRRGQNRSARIAKQL | 146 |
| RRRRRRRRNGEPRTPRRGQNRSARIAKQL | 147 |
| RRRRRRRRNGDPRDPRRG | 148 |
| RRRRRRRRNGEPRDPRRG | 149 |
| RRRRRRRRNGDPREPRRG | 150 |
| RRRRRRRRNGEPREPRRG | 151 |
| RRRRRRRRNGSPRDPRRG | 152 |
| RRRRRRRRNGSPREPRRG | 153 |
| RRRRRRRRNGDPRTPRRG | 154 |
| RRRRRRRRNGEPRTPRRG | 155 |
| RRRRRRRRNGDPRDPRRGQNRS | 156 |
| RRRRRRRRNGEPRDPRRGQNRS | 157 |
| RRRRRRRRNGDPREPRRGQNRS | 158 |
| RRRRRRRRNGEPREPRRGQNRS | 159 |
| RRRRRRRRNGSPRDPRRGQNRS | 160 |
| RRRRRRRRNGSPREPRRGQNRS | 161 |
| RRRRRRRRNGDPRTPRRGQNRS | 162 |
| RRRRRRRRNGEPRTPRRGQNRS | 163 |
| RRRRRRRRNGDPRDPRRGQNRSARI | 164 |
| RRRRRRRRNGEPRDPRRGQNRSARI | 165 |
| RRRRRRRRNGDPREPRRGQNRSARI | 166 |
| RRRRRRRRNGEPREPRRGQNRSARI | 167 |
| RRRRRRRRNGSPRDPRRGQNRSARI | 168 |
| RRRRRRRRNGSPREPRRGQNRSARI | 169 |
| RRRRRRRRNGDPRTPRRGQNRSARI | 170 |
| RRRRRRRRNGEPRTPRRGQNRSARI | 171 |
| RRRRRRRRNGDPRDPRRGQNRSARIAK | 172 |
| RRRRRRRRNGEPRDPRRGQNRSARIAK | 173 |
| RRRRRRRRNGDPREPRRGQNRSARIAK | 174 |
| RRRRRRRRNGEPREPRRGQNRSARIAK | 175 |
| RRRRRRRRNGSPRDPRRGQNRSARIAK | 176 |
| RRRRRRRRNGSPREPRRGQNRSARIAK | 177 |
| RRRRRRRRNGDPRTPRRGQNRSARIAK | 178 |
| RRRRRRRRNGEPRTPRRGQNRSARIAK | 179 |

In some cases, a polypeptide provided herein that includes SEQ ID NO:1 (NGX₁PRX₂PR, where X₁ is D or E, and X₂ is D or E) with an N-terminal and/or C-terminal cell targeting sequence (e.g., SEQ ID NO:91) can include one or more peptidase cleavage sites located between the cell targeting sequence(s) and SEQ ID NO:1. For example, using SEQ ID NO:92 as an example, a polypeptide can be designed to include one or more peptidase cleavage sites at the position marked with an “X”: RRRRRRRRXNGDPRDPR (SEQ ID NO:180). Any appropriate peptidase cleavage site can be used. An example of a peptidase cleavage site that can be used as described herein include, without limitation, an AFK sequence of D amino acids, which is a tripeptide recognized by plasmin. For example, a polypeptide having SEQ ID NO:180 where the X represents AFK can have the following amino acid sequence: RRRRRRRRAFKNGDPRDPR (SEQ ID NO:181). In these cases, cleavage at the cleavage site via a protease within a cell can liberate the peptide containing SEQ ID NO:1 from the amino acid sequence of an N-terminal and/or C-terminal cell targeting sequence. As described herein, local tumor injection and/or use of nanoparticles that incorporate one or more tumor-specific antibodies to direct a polypeptide provided herein (e.g., a polypeptide having an amino acid sequence set forth in any of the sequences of Table 1). See, e.g., Cully, Nature Rev. Drug Discovery, 15:231 (2016); and Davis, Nature Rev. Drug Discovery, 7:771-82 (2008)). As also described herein, viral vectors such as adenoviral vectors can be used to deliver nucleic acid designed to express a polypeptide provided herein (e.g., a polypeptide having an amino acid sequence set forth in any of the sequences of Table 1) to cells (e.g., cancer cells).

A polypeptide provided herein (e.g., a polypeptide having the amino acid sequence as set forth in any one of SEQ ID NOs:1-179) can be a substantially pure polypeptide. As used herein, the term “substantially pure” with reference to a polypeptide means that the polypeptide is substantially free of other polypeptides, lipids, carbohydrates, and nucleic acid. In some cases, a substantially pure polypeptide can be a polypeptide that is at least 60 percent pure or is any chemically synthesized polypeptide. A substantially pure polypeptide can be at least about 60, 65, 70, 75, 80, 85, 90, 95, or 99 percent pure. Typically, a substantially pure polypeptide will yield a single major band on a non-reducing polyacrylamide gel.

A polypeptide provided herein (e.g., a polypeptide having the amino acid sequence as set forth in any one of SEQ ID NOs:1-179) can be produced using any suitable method including, without limitation, solid phase synthesis, manual techniques, or automated techniques such as those involving the use of an Applied BioSystems (Foster City, Calif.) Peptide Synthesizer, a Biosearch Inc. (San Rafael, Calif.) automatic peptide synthesizer, a Biotage peptide synthesis instrument, or a CSBio Peptide Synthesizer. In some cases, a polypeptide provided herein can be produced recombinantly using nucleic acid as described herein.

In some cases, a polypeptide provided herein (e.g., a polypeptide having the amino acid sequence as set forth in any one of SEQ ID NOs:1-179) can be prepared to include one or more salt, ester, amide, N-acyl, and/or O-acyl moieties. For example, salts of carboxyl groups of a polypeptide provided herein can be prepared by contacting the polypeptide with one or more equivalents of a desired base such as, for example, a metallic hydroxide base (e.g., sodium hydroxide), a metal carbonate or bicarbonate base (e.g., sodium carbonate or sodium bicarbonate), or an amine base (e.g., triethylamine or triethanolamine). Acid addition salts of a polypeptide provided herein can be prepared by contacting the polypeptide with one or more equivalents of an inorganic or organic acid (e.g., hydrochloric acid). Esters of carboxyl groups of a polypeptide provided herein can be prepared using any suitable means for converting a carboxylic acid or precursor to an ester. For example, one method for preparing esters of a polypeptide provided herein, when using the Merrifield synthesis technique, is to cleave the completed polypeptide from the resin in the presence of the desired alcohol under either basic or acidic conditions, depending upon the resin. The C-terminal end of the polypeptide then can be directly esterified when freed from the resin, without isolation of the free acid. Amides of a polypeptide provided herein can be prepared using techniques for converting a carboxylic acid group or precursor to an amide. One method for amide formation at the C-terminal carboxyl group includes cleaving the polypeptide from a solid support with an appropriate amine, or cleaving in the presence of an alcohol, yielding an ester, followed by aminolysis with the desired amine. N-acyl derivatives of an amino group of a polypeptide provided herein can be prepared by utilizing an N-acyl protected amino acid for the final condensation, or by acylating a protected or unprotected polypeptide. O-acyl derivatives can be prepared, for example, by acylation of a free hydroxy peptide or peptide resin. Either acylation can be carried out using a standard acylating reagent such as an acyl halide, anhydride, and/or acyl imidazole. Both N- and O-acylation can be carried out together, if desired.

In some cases, a polypeptide provided herein (e.g., a polypeptide having the amino acid sequence as set forth in any one of SEQ ID NOs:1-179) can be modified by linkage to a polymer such as polyethylene glycol (PEG), or by fusion to another polypeptide such as albumin. For example, one or more PEG moieties can be conjugated to a polypeptide provided herein via lysine residues or other linkages. Linkage to PEG or another suitable polymer, or fusion to albumin or another suitable polypeptide, can result in a modified polypeptide having an increased half-life as compared to an unmodified polypeptide. Without being bound by a particular mechanism, an increased serum half-life can result from reduced proteolytic degradation, immune recognition, or cell scavenging of the modified polypeptide. Any appropriate method can be used to modify a polypeptide provided herein by linkage to PEG (also referred to as “PEGylation”) or other polymers including, without limitation, those described elsewhere (U.S. Pat. No. 6,884,780; Cataliotti et al., Trends Cardiovasc. Med., 17:10-14 (2007); Veronese and Mero, BioDrugs, 22:315-329 (2008); Miller et al., Bioconjugate Chem., 17:267-274 (2006); and Veronese and Pasut, Drug Discov. Today, 10:1451-1458 (2005)). Examples of methods for modifying a polypeptide provided herein by fusion to albumin include, without limitation, those described elsewhere (U.S. Patent Publication No. 2004/0086976, and Wang et al., Pharm. Res., 21:2105-2111 (2004)).

This document also provides isolated nucleic acid molecules encoding a polypeptide provided herein as well as expression vectors containing such nucleic acids and host cells containing such nucleic acids and/or expression vectors. As used herein, the term “nucleic acid” refers to both RNA and DNA, including cDNA, genomic DNA, and synthetic (e.g., chemically synthesized) DNA. A nucleic acid molecule can be double-stranded or single-stranded (i.e., a sense or an antisense single strand). Nucleic acids include, for example, cDNAs encoding a polypeptides provided herein.

An “isolated nucleic acid” as used herein refers to a nucleic acid that is separated from other nucleic acid molecules that are present in a vertebrate genome, including nucleic acids that normally flank one or both sides of the nucleic acid in a vertebrate genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

Isolated nucleic acid molecules can be produced using standard techniques, including, without limitation, common molecular cloning and chemical nucleic acid synthesis techniques. For example, polymerase chain reaction (PCR) techniques can be used to obtain an isolated nucleic acid containing a nucleotide sequence that encodes a polypeptide provided herein (e.g., a polypeptide having the amino acid sequence as set forth in any one of SEQ ID NOs:1-179). PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis, Genetic Engineering News, 12:1 (1992); Guatelli et al., Proc. Natl. Acad. Sci. USA, 87:1874-1878 (1990); and Weiss, Science, 254:1292 (1991)).

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids (e.g., nucleic acids encoding a polypeptide provided herein) also can be obtained by mutagenesis. For example, a reference sequence can be mutated using standard techniques including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992.

Sources of nucleotide sequences from which nucleic acid molecules encoding a polypeptide provided herein, or the nucleic acid complement thereof, can be obtained include total or polyA⁺ RNA from any eukaryotic source, including mammalian (e.g., human, rat, mouse, canine, bovine, equine, ovine, caprine, or feline) cellular source from which cDNAs can be derived. Other sources of the nucleic acid molecules include genomic libraries derived from any eukaryotic cellular source, including mammalian sources.

Nucleic acid molecules encoding a polypeptide provided herein can be identified and isolated using molecule cloning techniques, e.g., as described by Sambrook et al., Molecular Cloning: A Laboratory Manual, Cold Spring Harbor Laboratory Press, NY (1989). For example, reverse-transcriptase PCR (RT-PCR) can be used to isolate and clone cDNAs from isolated RNA that contains RNA sequences of interest (e.g., total RNA isolated from human tissue). Other approaches to identify, isolate, and clone cDNAs encoding a polypeptide provided herein include, for example, screening cDNA libraries.

Vectors containing nucleic acids such as those described herein also are provided. A “vector” is a replicon, such as a plasmid, phage, or cosmid, into which another DNA segment may be inserted so as to bring about the replication of the inserted segment. An “expression vector” is a vector that includes one or more expression control sequences, and an “expression control sequence” is a DNA sequence that controls and regulates the transcription and/or translation of another DNA sequence.

In expression vectors, a nucleic acid (e.g., a nucleic acid encoding a polypeptide provided herein) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are available commercially. In some cases, a viral vector such as a pTsin lentiviral vector can be designed to encode and express a polypeptide provided herein (e.g., a polypeptide having the amino acid sequence as set forth in any one of SEQ ID NOs:1-179).

An expression vector including a nucleic acid sequence that encodes a polypeptide provided herein (e.g., a polypeptide having the amino acid sequence as set forth in any one of SEQ ID NOs:1-179) can include a tag sequence designed to facilitate subsequent manipulation or localization of the expressed nucleic acid sequence (e.g., for purification). In some cases, tag sequences such as green fluorescent protein (GFP), glutathione S-transferase (GST), polyhistidine, c-myc, hemagglutinin, or Flag™ tag (Kodak, New Haven, Conn.) sequences can be expressed as a fusion with the encoded polypeptide. Such tags can be inserted anywhere within the polypeptide including at either the carboxyl or amino terminus.

This document also provides host cells containing a nucleic acid or vector provided herein. The term “host cell” is intended to include prokaryotic and eukaryotic cells into which a recombinant nucleic acid or vector (e.g., an expression vector) can be introduced. As used herein, “transformed” and “transfected” encompass the introduction of a nucleic acid molecule (e.g., a vector) into a cell by one of a number of techniques. In some cases, host cells can be transformed or transfected use methods described elsewhere (e.g., Sambrook et al., Molecular Cloning: A Laboratory Manual (2nd edition), Cold Spring Harbor Laboratory, New York (1989)). For example, calcium phosphate precipitation, electroporation, heat shock, lipofection, microinjection, and viral-mediated nucleic acid transfer can be used introduce nucleic acid encoding a polypeptide described herein into cells. In addition, naked DNA can be delivered directly to cells in vivo as described elsewhere (U.S. Pat. Nos. 5,580,859 and 5,589,466).

As described herein, the document also provides methods for reducing PD-L1 expression within cancer cells, methods for treating cancer, and/or methods for increasing the effectiveness that another cancer treatment method and/or cancer agent exhibits against cancer within a mammal (e.g., a human). In some cases, this document provides methods and materials for using compositions (e.g., compositions containing polypeptide provided herein) to reduce PD-L1 expression within cancer cells. This document also provides methods and materials for using compositions (e.g., compositions containing polypeptide provided herein) in combination with other cancer treatment methods or agents to increase the effectiveness exhibited against the cancer within a mammal (e.g., a human).

Any appropriate mammal (e.g., a human) having cancer can be treated as described herein. For example, a human having cancer can be treated using a composition containing a polypeptide provided herein (e.g., a polypeptide having the amino acid sequence as set forth in any one of SEQ ID NOs:1-179) and/or a nucleic acid encoding a polypeptide provided herein. Other examples of mammals that can be treated as described herein include, without limitation, non-human primates, monkeys, dogs, cats, horses, cows, pigs, sheep, rabbits, mice, and rats.

Any type of cancer can be treated as described herein. For example, prostate cancer, pancreatic cancer, lung cancer, liver cancer, or breast cancer can be treated as described herein. In some cases, a mammal (e.g., a human) having a PD-L1⁺ cancer can be treated with a composition provided herein (e.g., a composition containing a polypeptide provided herein and/or a nucleic acid encoding a polypeptide provided herein) to reduce the level of PD-L1 expression in cancer cells within the mammal and/or to reduce the number of cancer cells within the mammal. In some cases, a mammal (e.g., a human) suspected to develop cancer can be treated with a composition provided herein (e.g., a composition containing a polypeptide provided herein and/or a nucleic acid encoding a polypeptide provided herein) to slow or reduce the likelihood of the progression of and/or development of cancer.

Any appropriate method can be used to identify a mammal as having cancer or as being at risk for developing cancer. For example, tissue biopsy and/or imaging techniques (e.g., CT or MM) can be used to identify a human or other mammal as having cancer. In some cases, a human's family health history or genetic markers (e.g., BRCA1 and BRCA2) can be evaluated to determine if the human is at risk of developing cancer.

Once identified as having cancer or as being at risk for developing cancer, the mammal can be administered or instructed to self-administer a composition provided herein such as a composition formulated to include a polypeptide provided herein and/or formulated to include a nucleic acid provided herein (e.g., a viral vector containing a nucleic acid sequence encoding a polypeptide provided herein). In some cases, a composition containing a polypeptide provided herein administered to a mammal as described herein can include that polypeptide as the sole active ingredient. For example, a mammal having cancer or at risk for developing cancer can be administered a composition containing a polypeptide provided herein as the sole active ingredient. In some cases, a composition containing a nucleic acid provided herein administered to a mammal as described herein can include that nucleic acid as the sole active ingredient. For example, a mammal having cancer or at risk for developing cancer can be administered a composition containing a nucleic acid provided herein as the sole active ingredient.

A polypeptide provided herein or a nucleic acid encoding such a polypeptide can be used to reduce the level of PD-L1 expression by cancer cells within a mammal, to reduce the number of cancer cells within a mammal, and/or to increase the effectiveness of other cancer treatment methods and/or agents. In some cases, a composition containing a polypeptide provided herein (and/or a nucleic acid provided herein) can be administered to a mammal having cancer or at risk of developing cancer as a combination therapy with one or more additional cancer treatment methods or agents to treat cancer. In those cases where a polypeptide provided herein (and/or a nucleic acid provided herein) are used in combination with one or more additional cancer treatment methods and/or agents to treat cancer as described herein, the one or more additional cancer treatment methods and/or agents can be administered at the same time or independently. For example, a composition containing a polypeptide provided herein (and/or a nucleic acid provided herein) can be administered first, and the one or more additional cancer treatment methods and/or agents can be administered second, or vice versa. As described herein, a polypeptide provided herein (and/or a nucleic acid provided herein) such as a polypeptide having any one of the amino acid sequences set forth in SEQ ID NO:1-179 can be used to increase the effectiveness of another cancer treatment method or agent (when compared to the effectiveness observed with that other cancer treatment method or agent in the absence of the polypeptide (or nucleic acid)). Examples of other cancer treatment methods and/or agents that can be used as described herein include, without limitation, radiation treatments, chemotherapies such as camptothecin therapy, taxane therapy, kinase inhibitor therapy, and gemcitabine therapy, and surgery.

In some cases, a polypeptide provided herein (and/or a nucleic acid provided herein) can be formulated into a pharmaceutically acceptable composition for administration to a mammal having cancer or at risk of developing cancer. For example, a therapeutically effective amount of a polypeptide provided herein (and/or a nucleic acid provided herein) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing a polypeptide provided herein (and/or a nucleic acid provided herein) can be designed for oral, parenteral (e.g., subcutaneous, intramuscular, intravenous, or intradermal administration), or inhaled administration. When being administered orally, a pharmaceutical composition containing a polypeptide provided herein (and/or a nucleic acid provided herein) can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. Compositions for inhalation can be delivered using, for example, an inhaler, a nebulizer, and/or a dry powder inhaler. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a pharmaceutically acceptable composition including a polypeptide provided herein (and/or a nucleic acid provided herein) can be administered locally or systemically. For example, a composition containing a polypeptide provided herein (and/or a nucleic acid provided herein) can be administered systemically by an oral administration to or inhalation by a mammal (e.g., a human).

Effective doses can vary depending on the severity of the cancer and/or risk of cancer, the route of administration, the age and general health condition of the subject, excipient usage, the possibility of co-usage with other therapeutic treatments such as use of other agents, and the judgment of the treating physician.

An effective amount of a composition containing a polypeptide provided herein (and/or a nucleic acid provided herein) can be any amount that reduces the level of PD-L1 expression by cancer cells within a mammal and/or reduces the number of cancer cells within the mammal without producing significant toxicity to the mammal. For example, an effective amount of a polypeptide provided herein (and/or a nucleic acid provided herein) can be from about 0.5 mg/kg to about 50 mg/kg (e.g., from about 1 mg/kg to about 50 mg/kg, from about 2.5 mg/kg to about 50 mg/kg, from about 5 mg/kg to about 50 mg/kg, from about 0.5 mg/kg to about 25 mg/kg, from about 0.5 mg/kg to about 15 mg/kg, from about 0.5 mg/kg to about 10 mg/kg, from about 1 mg/kg to about 10 mg/kg, or from about 2.5 mg/kg to about 7.5 mg/kg). In one example, 800 mg of a polypeptide having any one of the amino acid sequences set forth in SEQ ID NOs:1-179 can be administered once a day to a 80 kg human. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of a composition containing a polypeptide provided herein (and/or a nucleic acid provided herein) can be any frequency that reduces the level of PD-L1 expression by cancer cells within a mammal and/or reduces the number of cancer cells within the mammal without producing significant toxicity to the mammal. For example, the frequency of administration can be from about three times a day to about once a day, from about once a week to about three times a month, from about twice a month to about six times a month, or from about twice a week to about once a month. The frequency of administration can remain constant or can be variable during the duration of treatment. A course of treatment with a composition containing a polypeptide provided herein (and/or a nucleic acid provided herein) can include rest periods. For example, a composition containing a polypeptide provided herein (and/or a nucleic acid provided herein) can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing a polypeptide provided herein (and/or a nucleic acid provided herein) can be any duration that reduces the level of PD-L1 expression by cancer cells within a mammal and/or reduces the number of cancer cells within the mammal without producing significant toxicity to the mammal. For example, the effective duration can vary from several days to several weeks, months, or years. In some cases, the effective duration for the treatment of cancer can range in duration from about one week to about 10 years. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment, the severity of one or more symptoms related to the condition being treated (e.g., cancer), and/or the number of cancer cells within a mammal can be monitored. Any appropriate method can be used to determine whether or not the severity of a symptom is reduced. For example, the severity of a symptom of cancer can be assessed using imaging techniques at different time points. In some cases, a scoring system can be used to assess the severity of cancer.

The invention will be further described in the following examples, which do not limit the scope of the invention described in the claims.

### EXAMPLES

### Example 1—RB Controls Tumor Immune Surveillance by Regulating NFκB Activity and PD-L1 Expression

### Cell Lines, Cell Culture, and Transfection

LNCaP, PC-3, 22Rv1, DU145, TRAMP-C2, and human embryonic kidney cell line 293T cell lines were purchased from American Type Culture Collection (Manassas). C4-2 CRPC cell line was purchased from Uro Corporation. PTEN-CaP8 murine PTEN-deficient CRPC cell line was obtained from Dr. Hong Wu at UCLA. The androgen-refractory LNCaP subline, RF, was established as described elsewhere (Murillo et al., Endocrinology, 142: 4795-4805 (2001)). LNCaP, PC-3, 22Rv1, and DU145 were cultured in RPMI 1640 supplemented with 10% FBS. LNCaP-RF were cultured in RPMI 1640 supplemented with 10% charcoal-stripped FBS (CSS). PTEN-CaP8 and 293T cells were maintained in DMEM supplemented with 10% FBS. Cells were cultured at 37° C. supplied with 5% CO₂. LAPC-4 cells were obtained from C. L. Sawyers and maintained in Iscove's Modified Dulbecco's Media with 10% FBS. RB-deficient mouse prostate epithelial cells (RB^{-/-} PrE) were obtained from M. L. Day and S. W. Hayward and maintained in RPMI 1640 containing 200 μg/mL G418, 5% FBS, 100 m/mL streptomycin, 100 U/mL penicillin, and 0.25 μg/mL amphotericin B. SKO(Ptenf/fRb1^{+/+}) and DKO cr (Ptenf/fRb1^{f/f}) was obtained from David W. Goodrich and maintained in DMEM supplemented with 2.5% charcoal-stripped FBS, 5 μg/mL of insulin/transferring/selenium (Collaborative Research), 10 μg/mL of bovine pituitary extract (Sigma), 10 μg/mL of epidermal growth factor (Collaborative Research), and 1 μg/mL of cholera toxin (Sigma). All cell lines were kept in a 37° C. incubator at 5% CO². Transfections were performed by using Lipofectamine 2000 (Thermo Fisher Scientific).

### Prostate Cancer Patient Samples and Tissue Microarray

The advanced prostate cancer dataset was generated from patients undergoing standard of care clinical biopsies at Mayo Clinic. A tissue microarray was constructed from the formalin-fixed, paraffin-embedded (FFPE) samples of metastatic prostate cancer, identified after a search of pathologic and clinical databases of archival tissues. The human tissue microarray (TMA) contained 157 cores (16 0.6 mm and 141 1.0 mm cores) resulting from 53 samples (20 bone metastases and 33 non-bone metastases) from 51 patients. FFPE tissue was used for IHC analysis. 145 cores were used for IHC data analysis when cores with lost tissue greater than 50% were excluded.

### RNA Interference

Lentivirus-based control and gene-specific small hairpin RNAs (shRNAs) were purchased from Sigma-Aldrich. Viral packaging plasmids (pEXQV and pVSV-G) and shRNA plasmid were transfected to 293T cells by using Lipofectamine 2000. After 24 hours, virus culture medium was replaced with DMEM supplemented with 10% FBS and 1:100 of sodium Pyruvate. 48 hours post transfection, medium was collected and added to various cancer cells supplemented with 12 μg/mL of polybrene. Cancer cells were harvested 48 hours after puromycin selection. shRNA sequence information are provided in Table 3.

TABLE 3
Sequences for shRNAs.

| | |
|---|---|
| shRB1-1 | 5′-CCGGGTGCGCTCTTGAGGTTGTAATCTCGAGATT |
| | ACAACCTCAAGAGCGCACTTTTTG-3′ |
| | (SEQ ID NO: 182) |
| shRB1-2 | 5′-CCGGCAGAGATCGTGTATTGAGATTCTCGAGAAT |
| | CTCAATACACGATCTCTGTTTTTG-3′ |
| | (SEQ ID NO: 183) |
| shRB1-3 | 5′-CCGGGACTTCTACTCGAACACGAATCTCGAGATT |
| | CGTGTTCGAGTAGAAGTCTTTTTG-3′ |
| | (SEQ ID NO: 184) |
| shCHD1-1 | 5′-CCGGTGATGAAGCACACCGATTAAACTCGAGTTT |
| | AATCGGTGTGCTTCATCATTTTTG-3′ |
| | (SEQ ID NO: 185) |
| shCHD1-2 | 5′-CCGGGCGGTTTATCAAGAGCTATAACTCGAGTTA |
| | TAGCTCTTGATAAACCGCTTTTT-3′ |
| | (SEQ ID NO: 186) |
| shCHD1-3 | 5′-CCGGCGGATTGAGGAGAAACGTAAACTCGAGTTT |
| | ACGTTTCTCCTCAATCCGTTTT-3′ |
| | (SEQ ID NO: 187) |
| shp65-1 | 5′-CCGGCGGATTGAGGAGAAACGTAAACTCGAGTTT |
| | ACGTTTCTCCTCAATCCGTTTT-3′ |
| | (SEQ ID NO: 188) |
| shp65-2 | 5′-CCGGCACCATCAACTATGATGAGTTCTCGAGAAC |
| | TCATCATAGTTGATGGTGTTTTT-3′ |
| | (SEQ ID NO: 189) |
| shp65-3 | 5′-CCGGGCCTTAATAGTAGGGTAAGTTCTCGAGAAC |
| | TTACCCTACTATTAAGGCTTTTT-3′ |
| | (SEQ ID NO: 190) |
| shCDK4-1 | 5′-CCGGATGACTGGCCTCGAGATGTACTCGAGTACA |
| | TCTCGAGGCCAGTCATCTTTTTG-3′ |
| | (SEQ ID NO: 191) |
| shCDK4-2 | 5′-CCGGACAGTTCGTGAGGTGGCTTTACTCGAGTAA |
| | AGCCACCTCACGAACTGTTTTTT-3′ |
| | (SEQ ID NO: 192) |
| shCDK4-3 | 5′-CCGGAGGACATATCTGGACAAGGCACTCGAGTGC |
| | CTTGTCCAGATATGTCCTTTTTT-3′ |
| | (SEQ ID NO: 193) |
| shCDK6-1 | 5′-CCGGTCTGGAGTGTTGGCTGCATATCTCGAGATA |
| | TGCAGCCAACACTCCAGATTTT-3′ |
| | (SEQ ID NO: 194) |
| shCDK6-2 | 5′-CCGGCATGAGATGTTCCTATCTTAACTCGAGTTA |
| | AGATAGGAACATCTCATGTTTTTTG-3′ |
| | (SEQ ID NO: 195) |
| shCDK6-3 | 5′-CCGGGAGAAGTTTGTAACAGATATCCTCGAGGAT |
| | ATCTGTTACAAACTTCTCTTTTTTG-3′ |
| | (SEQ ID NO: 196) |
| shIKK α-1 | 5′-CCGGGCAGATGACGTATGGGATATCCTCGAGGAT |
| | ATCCCATACGTCATCTGCTTTTTTG-3′ |
| | (SEQ ID NO: 197) |
| shIKK α-2 | 5′-CCGGCCAGATACTTTCTTTACTAAGCTCGAGCTT |
| | AGTAAAGAAAGTATCTGGTTTTTTG-3′ |
| | (SEQ ID NO: 198) |
| shIKK α-3 | 5′-CCGGGCTGCTCACAAGTTCTATTTCCTCGAGGAA |
| | ATAGAACTTGTGAGCAGCTTTTTTG-3′ |
| | (SEQ ID NO: 199) |
| shIKK β-1 | 5′-CCGGGCTGGTTCATATCTTGAACATCTCGAGATG |
| | TTCAAGATATGAACCAGCTTTTTG-3′ |
| | (SEQ ID NO: 200) |
| shIKK β-2 | 5′-CCGGGCTGGTTCATATCTTGAACATCTCGAGATG |
| | TTCAAGATATGAACCAGCTTTTT-3′ |
| | (SEQ ID NO: 201) |
| shIKK β-3 | 5′-CCGGCCAGCCAAGAAGAGTGAAGAACTCGAGTTC |
| | TTCACTCTTCTTGGCTGGTTTTT-3′ |
| | (SEQ ID NO: 202) |

### Quantitative RT-PCR

Total RNA was isolated using TRIzol reagent (Thermo Fisher Scientific). The NanoDrop 2000 spectrophotometer (Thermo Fisher Scientific) was used to assess RNA yield and quality. RNA was reversely transcribed using Superscript reverse transcriptase (Thermo Fisher Scientific) following manufacturer's instructions. Quantitative real-time PCR was performed by mixing cDNA, gene-specific primers, and IQ SYRB Green Supermix and detected by iCycler QTX detection system (Bio-Rad). The 2-ΔCt method was used to quantitate fold changes by normalizing to GAPDH. Primers for RT-qPCR are provided Table 4.

TABLE 4
Primers for RT-qPCR.

| Species | Gene | Forward (5′-3′) | Reverse (5′-3′) |
|---|---|---|---|
| Human | GAPDH | ACCCAGAAGACTGTGG | TTCAGCTCAGGGATGA |
| | | ATGG | CCTT |
| | | (SEQ ID NO: 203) | (SEQ ID NO: 204) |
| Human | β-actin | GACCTCTATGCCAACA | AGTACTTGCGCTCAGG |
| | | CAGT | AGGA |
| | | (SEQ ID NO: 205) | (SEQ ID NO: 206) |
| Human | TNF | GAGGCCAAGCCCTGGT | CGGGCCGATTGATCTC |
| | | ATG | AGC |
| | | (SEQ ID NO: 207) | (SEQ ID NO: 208) |
| Human | CCL20 | TGCTGTACCAAGAGTT | CGCACACAGACAACTT |
| | | TGCTC | TTTCTTT |
| | | (SEQ ID NO: 209) | (SEQ ID NO: 210) |
| Human | RB1 | TTTCTGCTTTTGCATT | GGAAGCAACCCTCCTA |
| | | CGTG | AACC |
| | | (SEQ ID NO: 211) | (SEQ ID NO: 212) |
| Human | PD-L1 | GGTGCCGACTACAAGC | AGCCCTCAGCCTGACA |
| | | GAAT | TGTC |
| | | (SEQ ID NO: 213) | (SEQ ID NO: 214) |
| Human | CXCL1 | AACAGCCACCAGTGAG | GAAAGCTTGCCTCAAT |
| | | CTTC | CCTG |
| | | (SEQ ID NO: 215) | (SEQ ID NO: 216) |
| Human | GADD45B | TGACAACGACATCAAC | GTGACCAGAGACAATG |
| | | ATC | CAG |
| | | (SEQ ID NO: 217) | (SEQ ID NO: 218) |
| Human | NR4A2 | GTCTCAGCTGCTCGAC | TTTTGCACTGTGCGCT |
| | | ACG | TAAA |
| | | (SEQ ID NO: 219) | (SEQ ID NO: 220) |
| Human | CD83 | TCCTGAGCTGCGCCTA | GCAGGGCAAGTCCACA |
| | | CAG | TCTT |
| | | (SEQ ID NO: 221) | (SEQ ID NO: 222) |
| Human | BIRC2 | TGTGGCCTGATGTTGG | GGTGACGAATGTGCAA |
| | | ATAAC | ATCTACT |
| | | (SEQ ID NO: 223) | (SEQ ID NO: 224) |
| Human | BIRC3 | ACGCAGCAATCGTGCA | CCTATAACGAGGTCAC |
| | | TTTTG | TGACGG |
| | | (SEQ ID NO: 225) | (SEQ ID NO: 226) |
| Human | GADD45α | TGAGCTGCTGCTACTG | TCCCGGCAAAAACAAA |
| | | GAGA | TAAG |
| | | (SEQ ID NO: 227) | (SEQ ID NO: 228) |
| Human | MCM3 | TCAAGCCTGTCCTGAC | CAGGTCCACAGTCTTG |
| | | ACAG | CTCA |
| | | (SEQ ID NO: 229) | (SEQ ID NO: 230) |
| Human | p107 | ATACGACTTGGCGAAT | GAGCGCTTCTTGGTGT |
| | | CAGG | AAGG |
| | | (SEQ ID NO: 231) | (SEQ ID NO: 232) |
| Mouse | Gapdh | AGGTTGTCTCCTGCGA | GGGTGGTCCAGGGTTT |
| | | CTTCA | CTTACT |
| | | (SEQ ID NO: 233) | (SEQ ID NO: 234) |
| Mouse | Pd-11 | AATGCTGCCCTTCAGA | ATAACCCTCGGCCTGA |
| | | TCAC | CATA |
| | | (SEQ ID NO: 235) | (SEQ ID NO: 236) |
| Mouse | Gadd45b | GCACTGCCTCCTGGTC | TGCCTCTGCTCTCTTC |
| | | AC | ACAG |
| | | (SEQ ID NO: 237) | (SEQ ID NO: 238) |
| Mouse | Gadd45α | TGAGCTGCTGCTACTG | TCCCGGCAAAAACAAA |
| | | GAGA | TAAG |
| | | (SEQ ID NO: 239) | (SEQ ID NO: 240) |
| Mouse | Birc2 | TGATGGTGGCTTGAGA | TGAATCTCATCAACAA |
| | | TGTTGGGA | ACTCCTGACCC |
| | | (SEQ ID NO: 241) | (SEQ ID NO: 242) |
| Mouse | Birc3 | TGTCAGCCAAGTTCAA | ATCTTCCGAACTTTCT |
| | | GCTG | CCAGGG |
| | | (SEQ ID NO: 243) | (SEQ ID NO: 244) |

### Plasmids and Reagents

pCMV4 p65, pCMV HA hRB-wt, and pCMV HA hRb delta CDK were purchased from Addgene (Cambridge, Mass.). Expression vectors for GST-FBP1 and GST-TRIM28 recombinant proteins were constructed using the pGEX-4T-1 backbone vector. RL S249A/T252A polypeptide, RL S249D/T252D polypeptide, and HA-RB1 661W mutants were generated with the KOD Plus Mutagenesis Kit (Toyobo) following the manufacturer's instructions. The following antibodies were used: RB (BD Biosciences, 554136, working dilution 1:1000), RB (Cell Signaling, 9309, working dilution 1:1000), p65 (Cell Signaling, 8242S, working dilution 1:1000), p65 (Cell Signaling, 6956, working dilution 1:1000), PD-L1 (Cell Signaling, 13684S, working dilution 1:1000), PD-L1 (Proteintech, 17952-1-AP, working dilution 1:1000), p50 (Cell Signaling, 135865, working dilution 1:1000), Phospho-RB (Ser795) (Cell Signaling, 93015, working dilution 1:1000), Phospho-RB (Ser249, Thr252) (Thermo scientific, 701059, working dilution 1:1000), P107 (Santa Cruz Biotechnology, SC-318, working dilution 1:1000), P130 (Santa Cruz Biotechnology, SC-317, working dilution 1:1000), ERK1/2 (Santa Cruz Biotechnology, sc-135900, working dilution 1:5000), anti-Flag (Sigma-Aldrich, F-3165, working dilution 1:3000), and anti-HA (Covance, MMS-101R, working dilution 1:3000), anti-light chain specific rabbit IgG secondary antibody (211-032-171), and anti-light chain specific mouse IgG secondary antibody (115-035-174) (Jackson Immuno Research laboratories). Palbociclib (PD0332991) was purchased from Selleckchem. Helenalin was purchased from Abcam.

### Co-Immunoprecipitation (Co-IP)

Cells were harvested and lysed by IP buffer (50 mM Tris-HCl, pH 7.4, 150 mM NaCl, 1% Triton X-100, 1% sodium deoxycholate, and 1% protease inhibitor cocktails) on ice for more than 30 minutes. Cell lysate was centrifuged for 15 minutes at 13,000 rpm at 4° C., and the supernatant was incubated with primary antibodies and protein A/G agarose beads (Thermo Fisher Scientific) with rotating at 4° C. overnight. The next day, the beads were washed at least six times with IP buffer on ice, and then subjected to western blotting analysis.

### Western Blotting

Cells were harvested and lysed by IP buffer, and the supernatant was quantified by BCA protein quantification assay. Equal amounts of protein sample were added into 4× sample buffer and boiled for 5 minutes. The sample was subjected to SDS-PAGE analysis and transferred to nitrocellulose membrane. The membrane was blocked by 5% milk for 1 hour at room temperature and incubated with primary antibody at 4° C. overnight. The next day, the membrane was washed three times with 1×TBST and incubated with horseradish peroxidase-conjugated secondary antibodies for 1 hour at room temperature. The protein bands were visualized by SuperSignal West Pico Stable Peroxide Solution (Thermo Fisher Scientific).

### Immunofluorescent Cytochemistry

Immunofluorescent cytochemistry was performed as described elsewhere (Pan et al., EMBO J., 36:995-1010 (2017)). Briefly, cells were fixed in 4% paraformaldehyde for 15 minutes. After washed in PBS three times, fixed cells were permeabilized with 0.2% Triton X-100 for 20 minutes, washed in PBS, and then blocked in PBS supplemented with 10% goat serum. Cells were incubated with indicated primary antibody at 4° C. overnight. After washed three times with PBS, cells were incubated with secondary antibody that was conjugated with Alexa Fluor (Thermo Fisher Scientific) for 1 hour at room temperature. After washed three times with PBS, cells were counterstained with Vectashield (Vector Laboratories) containing DAPI (4′, 6-diamidino-2-phenylindole). Images were captured using Zeiss laser confocal microscope (LSM780).

### Glutathione S-Transferase (GST) Pull-Down Assay

Cells were lysed with IP buffer (50 mM Tris-HCl, pH 7.4, 150 mM NaCl, 1% Triton X-100, 1% sodium deoxycholate, and 1% protease inhibitor cocktails) on ice for more than 30 minutes. GST fusion proteins were immobilized on glutathione-Sepharose beads (GE Healthcare Lifesciences). After washing with lysis buffer, the beads were incubated with cell lysates overnight at 4° C. overnight. The beads were then washed six times with binding buffer and re-suspended in sample buffer. The bound proteins were subjected to western blotting analysis.

### In Vitro Kinase Assay

Plasmid DNA (V5-CDK4 or pCMV4 p65) was add to the TNT® T7 Quick Master Mix and add 1 μL methionine (1 mM), by following the manufacturer's instruction of TNT® Quick Coupled Transcription/Translation Systems (Promega). GST or GST-RB-N recombinant proteins (GST-RB-N, GST-RB-N S249A/T252A, or GST-RB-N ΔS249/T252) were immobilized on glutathione-Sepharose beads. After washing with PBS, the beads were incubated with in vitro transcribed and translated CDK4, human recombinant CDK6/Cyclin D3 (Promega), and reaction buffer (40 mM Tris 7.5; 20 mM MgCl₂; 0.1 mg/mL BSA; 50 μM DTT) at room temperature for 60 minutes. After washing with PBS, the beads were incubated with in vitro transcribed and translated p65 for 4 hours. The beads were then washed six times with PBS and re-suspended in sample buffer. The bound proteins were subjected to western blotting analysis.

### RNA-Seq and Data Analysis

PC-3 cells were treated with or without Palbociclib (5 μM) for 24 hours or PC-3 cells were infected with lentivirus expressing control shRNA or DUB3-specific shRNA following puromycin selection after 48 hours infection. Total RNA was isolated from cells using the RNeasy Plus Mini Kit (Qiagen). High-quality (Agilent Bioanalyzer RIN>7.0) total RNA was employed for the preparation of sequencing libraries using the Illumina TruSeq Stranded Total RNA/Ribo-Zero Sample Prep Kit. A total of 500-1,000 ng of riboRNA-depleted total RNA was fragmented by RNase III treatment at 37° C. for 10-18 minutes, and RNase III was inactivated at 65° C. for 10 minutes. Size selection (50- to 150-bp fragments) was performed using the FlashPAGE denaturing PAGE-fractionator (Thermo Fisher Scientific) before ethanol precipitation overnight. The resulting RNA was directionally ligated, reverse-transcribed, and treated with RNase H.

### Chromatin Immunoprecipitation (ChIP) and ChIP-reChIP Assay

ChIP was performed as described elsewhere (Zhao et al., Cell Reports, 15:599-610 (2016)). For ChIP-reChIP assay DNA, cell lysates were sonicated and subjected to immunoprecipitation using p65 antibody. After being washed by RIPA buffer (50 mM Hepes-KOH, pH 7.6, 500 mM LiCl, 1 mM EDTA, 1% NP-40, and 0.7% Na-Deoxycholate), the protein-DNA complexes were eluted by elution buffer (10 mM Tris, 1 mM EDTA, 2% SDS, and 20 mM DTT, PH 7.5) for 30 minutes at 37° C. Then, the supernatant was diluted 20 times and subjected to the second ChIP using IgG or RB antibodies (Li et al., Nature, 513:251-255 (2014)). DNA pulled down by antibodies or nonspecific IgG was amplified by real-time PCR. The ChIP primers are provided in Table 5.

TABLE 5
ChIP-qPCR primer sequences.

| Species | | | |
|---|---|---|---|
| ChIP | | | |
| target | Gene | Forward (5′-3′) | Reverse (5′-3′) |
| Human, | GAPDH | GAAAGGCAATCCCAGA | TCTAGCTAAAAGCCGG |
| p65 | | AAGG | TTGC |
| | | (SEQ ID NO: 245) | (SEQ ID NO: 246) |
| Human, | PD-L1 | GGACACCAACACTAGA | CTGCCCAAGGCAGCAA |
| p65 | | TACCTAAACTG | AT |
| | | (SEQ ID NO: 247) | (SEQ ID NO: 248) |
| Human, | GADD45B | CCAGCAGAACTTGGGA | GCGAATGCCAGAAAAG |
| p65 | | AAGG | AAAA |
| | | (SEQ ID NO: 249) | (SEQ ID NO: 250) |
| Human, | NR4A2 | CAGGTAGTACGCACCT | CTGGGACAGGAAAAGG |
| p65 | | GGAG | GAGT |
| | | (SEQ ID NO: 251) | (SEQ ID NO: 252) |
| Human, | CD83 | GCCTAAGCGGGACTAG | CTGCCACGAGCTGCAG |
| p65 | | GAG | AG |
| | | (SEQ ID NO: 253) | (SEQ ID NO: 254) |

### Nuclear Extracts Preparation and Electrophoretic Mobility Shift Assay

Cells were collected and resuspend cell pellet in 1 mL of Buffer A (10 mM Hepes-KOH, pH 7.9, 1.5 mM MgCl2, 10 mM KCl, and 0.1% NP-40) to lyse the cells on ice for 10 minutes. Centrifuge sample at 6,500 rpm 4° C. for 3 minutes to pellet the nuclei. Wash nuclei pellet with Buffer A. Spin samples 3,500 rpm for 5 minutes at 4° C. Cell pellet was lysed by IP buffer (50 mM Tris-HCl, pH 7.4, 150 mM NaCl, 1% Triton X-100, 1% sodium deoxycholate, and 1% protease inhibitor cocktails) on ice for more than 30 minutes. Protein concentration was determined by BCA protein quantification assay.

DNA oligonucleotide was labeled with biotin by following the manufacturer's instruction of Pierce™ Biotin 3′ End DNA Labeling Kit (Thermo Fisher Scientific). Complimentary 3′ end-labeled oligos were annealed prior to use. Five micrograms nuclear extracts were added to the binding reaction system (10× Binding Buffer, 1 μg/μL Poly (dI·dC), 1% NP-40, and 100 mM MgCl2) and incubated at room temperature for 5 minutes. Then, biotin-labeled doublestrand PD-L1-prom oligonucleotide DNA (5′-GGTCA-GGAAAGTCCAACGCC-3′; SEQ ID NO:255) was added to binding reactions and incubated at room temperature for 20 minutes. Competitive assays also were performed by addition of 200-fold excess of unlabeled probe (5′-GGTCAGGAAAGTCCAACGCC-3′; SEQ ID NO:256) or unlabeled mutant probe (5′-GGTCATTCCCTGAACACGCC-3′; SEQ ID NO:257) to nuclear extracts at room temperature for 5 minutes before the addition of the labeled probe. The shift was performed by following the manufacturer's instruction of LightShift™ Chemiluminescent EMSA Kit (Thermo Fisher Scientific). Briefly, bound complexes were separated on 6% nondenaturating polyacrylamide gels and transferred to nylon membrane. The transferred DNA was crosslinked to membrane using UV-light and detected by chemiluminescence.

### PTEN-CaP8 Mice Tumor Models

6-week-old C57BL/6 mice (Jackson Lab) were used for animal experiments. All mice were housed in standard conditions with a 12-hour light/dark cycle and access to food and water ad libitum. PTEN-CaP8 cells (5×10⁶) infected with lentivirus expression Tsin control or Tsin-RL S249D/T252D polypeptide (in 50 μL 1×PBS plus 50 μL Matrigel (BD Biosciences)) were injected s.c. into the right flank of mice. The volume of xenografts was measured every other day and calculated using the formula L×W2×0.5. After xenografts reached a size of approximately 40 mm³, mice were randomized into different groups and treated with IR (12 Gy initiated at day 1) and anti-PD-L1 (200 i.p., given at days 0, 3, 6, and 9) alone or combination of IR. Mice were euthanized, and tumors collected from all animals once tumors reached a volume of 200 mm³.

### Flow Cytometry Analysis

PC-3 cells were harvested and washed with PBS. Cells were fixed in 4% paraformaldehyde for 15 minutes. After washed with PBS, cells were incubated with ice-cold 100% methanol 30 minutes on ice. Cells were washed with PBS and incubated with PD-L1 antibody (Cell Signaling, 13684S, working dilution 1:400) for 1 hour at room temperature. Then, cells were washed with PBS and incubated with secondary antibody that was conjugated with Alexa Fluor (Thermo Fisher Scientific) for 1 hour at room temperature. After washed three times with PBS, cells were resuspended with PBS and analyzed on flow cytometer.

For flow cytometry analysis of mouse tissue samples, tumors were cut into small pieces and digested with 2 mg/mL collagenase (Sigma) in DMEM for 1 hour at 37° C. Cells were filtered through 70 μm nylon strainer and resuspended in red blood cell lysis buffer (Biolegend) for 3 minutes at room temperature. The cells were then suspended in PBS with 2% BSA and co-stained with the following antibodies: CD45 (Biolegend, 103112, APC conjugated), CD4 (Biolegend, 100510, FITC conjugated), CD8 (Biolegend, 100708, PE conjugated), CD11b (Biolegend, 101212, APC conjugated), and Gr1 (Biolegend, 108406, FITC conjugated). After incubated with antibody for 30 minutes, cells were washed with PBS and analyzed on a flow cytometer.

### Statistical Analysis

Statistical analyses were performed with two sided paired Student t test for single comparison and one-way ANOVA and a post hoc test for multiple comparisons. P values<0.05 are considered statistically significant. All values were expressed as means±SD. Pearson's product-moment correlation was used to calculate the correlation between PD-L1 and pRB-S249/T252 staining index in prostate cancer TMAs.

### Results

RB Suppresses PD-L1 Expression at the mRNA Level

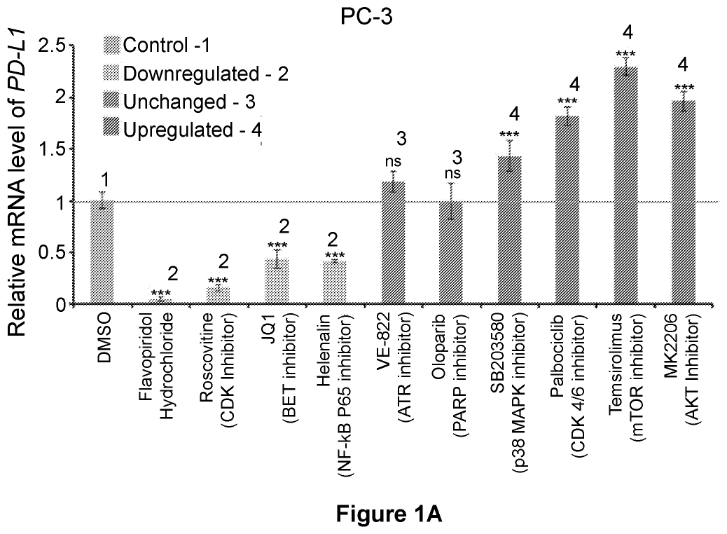

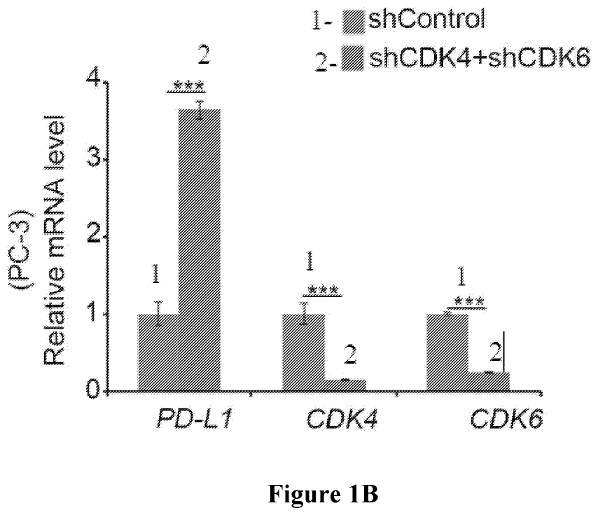

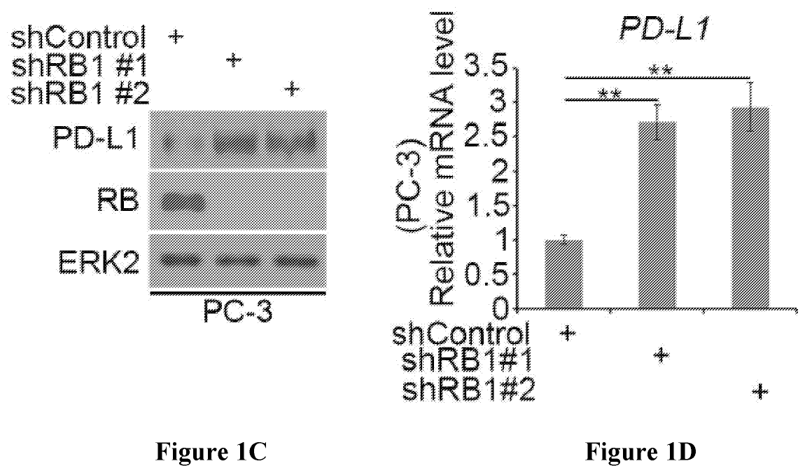

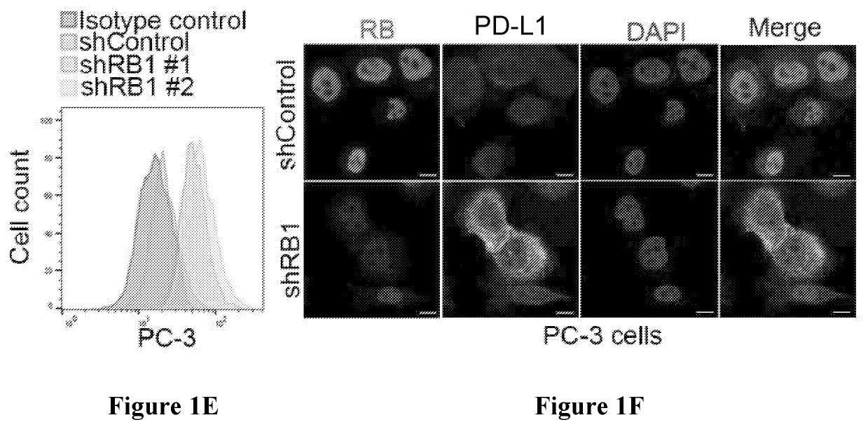

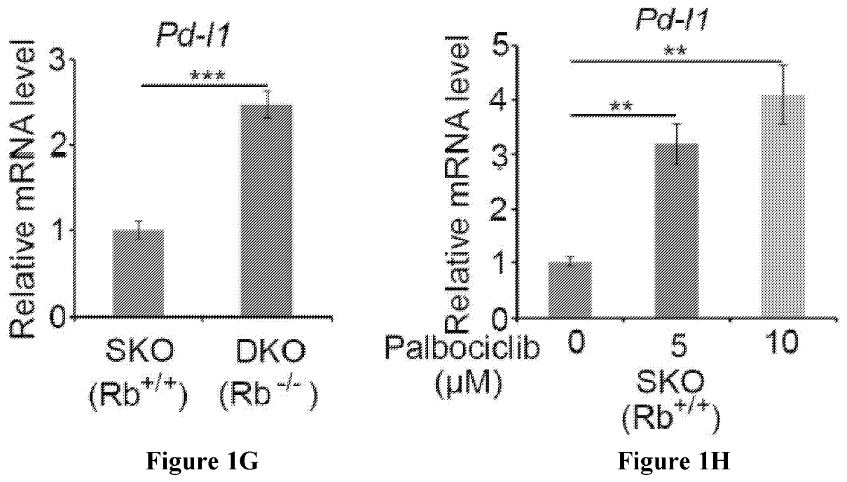

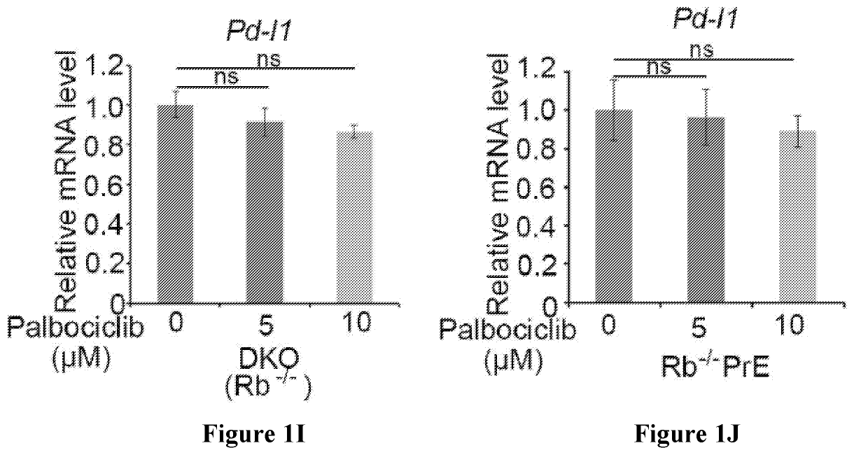

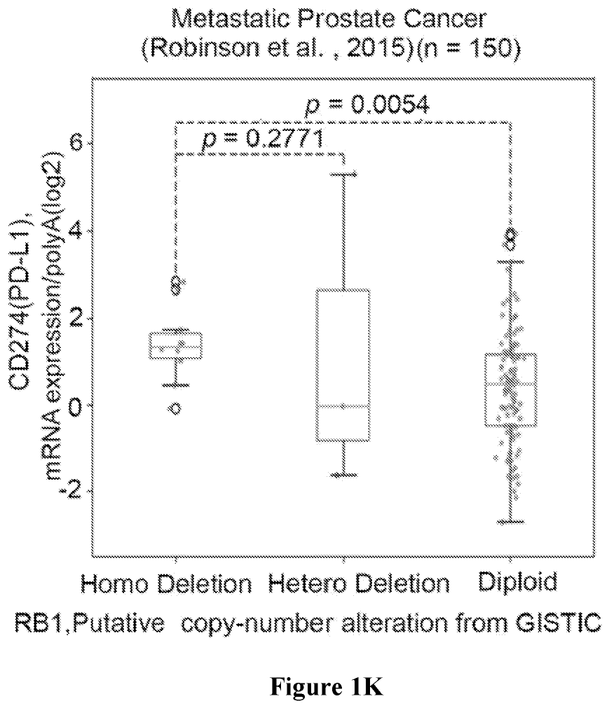

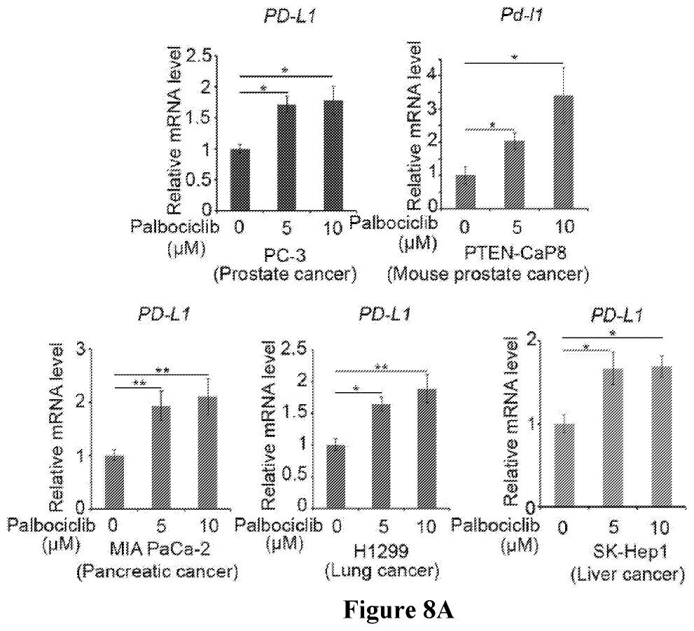

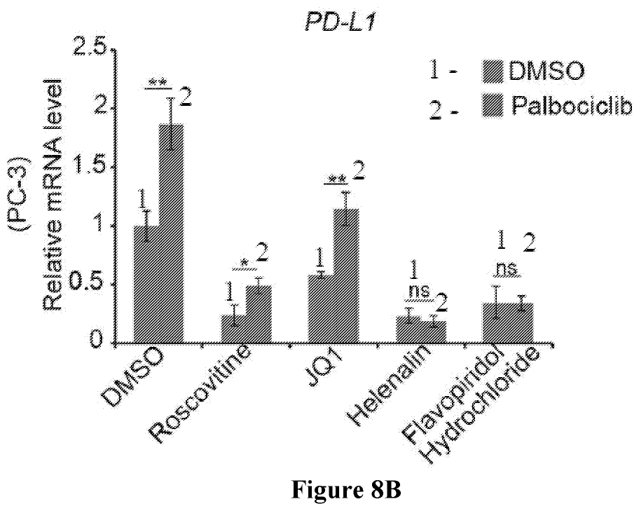

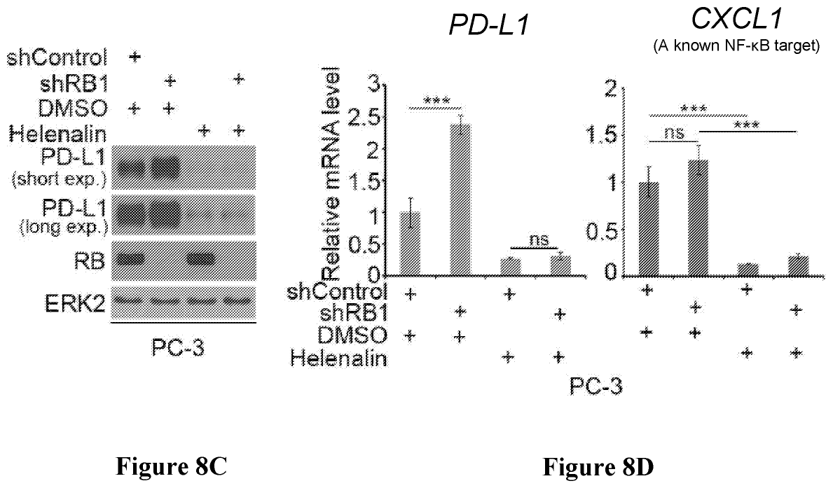

Increasing evidence indicates that aberrant elevation of PD-L1 allows cancer cells to escape from immune surveillance. An array of small molecule inhibitors that are either in clinical use or potential agents for cancer therapy was surveyed to determine which compounds could trigger undesired upregulation of PD-L1 expression. In good agreement with previous reports (Dorand et al., Science, 353:399-403 (2016); and Zhu et al., Cell Reports, 16:2829-2837 (2016)), treatment with flavopiridol (effective inhibitor of CDK9 of the P-TEFb complex), roscovitine (nonselective inhibitor of CDKs 1, 2, 5, 7, and 9), and JQ1 (BET protein inhibitor), resulted in dramatic downregulation of PD-L1 mRNA in PC-3 prostate cancer cells (FIG. 1A). In contrast, while little or no change in PD-L1 expression was detected in cells treated with some compounds, several other inhibitors including CDK4/6 inhibitor palbociclib significantly increased PD-L1 expression at the mRNA level (FIGS. 1A and 8A). Palbociclib also markedly induced PD-L1 mRNA expression in other human and mouse cancer cell lines of different tissue origins (FIG. 8A). Co-knockdown of CDK4 and CDK6 by small hairpin RNAs (shRNAs) also markedly increased PD-L1 mRNA expression (FIG. 1B). These results indicate that CDK4/6 inhibition upregulates PD-L1 at the mRNA level regardless the cancer type examined.

Given that RB is a major downstream effector of CDK4/6 signaling, the following was performed to determine whether RB regulates PD-L1 expression. Transient knockdown of endogenous RB1 gene by two independent shRNAs invariably increased PD-L1 expression at the levels of mRNA, protein, and cell surface (FIGS. 1C-1F). Pd-L1 mRNA expression also was upregulated by Rb1 homozygous deletion in Pten-null mouse prostate tumor cells compared to Rb1 wild-type counterparts (FIG. 1G). Different from the results in RB-proficient cell lines (FIGS. 1A, 1H, and 8A), palbociclib treatment failed to induce Pd-L1 mRNA expression in Rb1-deficient mouse prostate cancer and non-cancerous cell lines (FIGS. 1I and 1J), arguing that RB is required for CDK4/6 inhibition-induced PD-L1 mRNA upregulation. The findings from cell lines were fully supported by the data in a cohort of metastatic castration-resistant prostate cancer (mCRPC) patients that PD-L1 mRNA levels were significantly higher in RB1-homozygously deleted tumors than those in tumors without RB1 gene deletion (FIG. 1K).

RB Phosphorylation by CDK4/6 Enhances RB Interaction with p65 NFκB Protein

Nuclear factor-κB (NFκB) plays a role in regulating PD-L1 transcription in response to various stimuli in different cancer types (Peng Jin et al., Cancer Research, 75:5034-5045 (2015); and Bouillez et al., Oncogene, 36:4037-4046 (2017)). Treatment of PC-3 cells with the NFκB inhibitor helenalin decreased PD-L1 mRNA expression (FIG. 1A). With the goal to limit PD-L1 upregulation-induced immune evasion of cancer cells, the following was performed to determine whether any of PD-L1 expression-inhibitory compounds used, including roscovitine, JQ1, flavopiridol, and helenalin, have the ability to block specifically palbociclib-induced PD-L1 expression (FIG. 1A). Apart from roscovitine and JQ1, flavopiridol or helenalin treatment completely abolished palbociclib-induced elevation of PD-L1 (FIG. 8B).

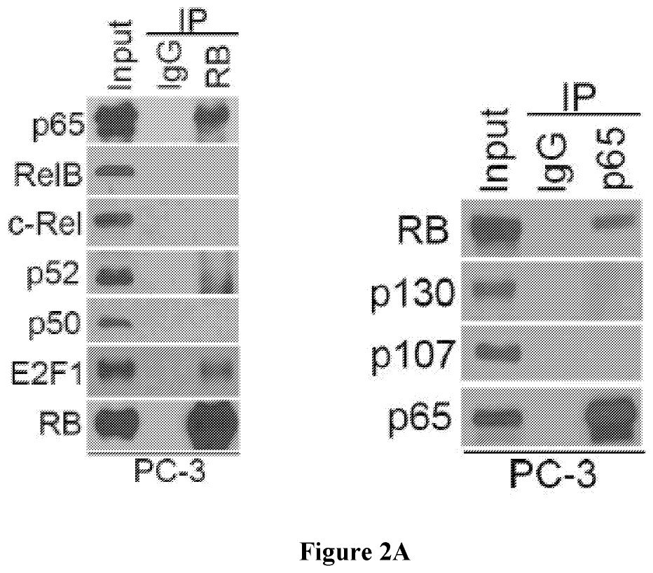

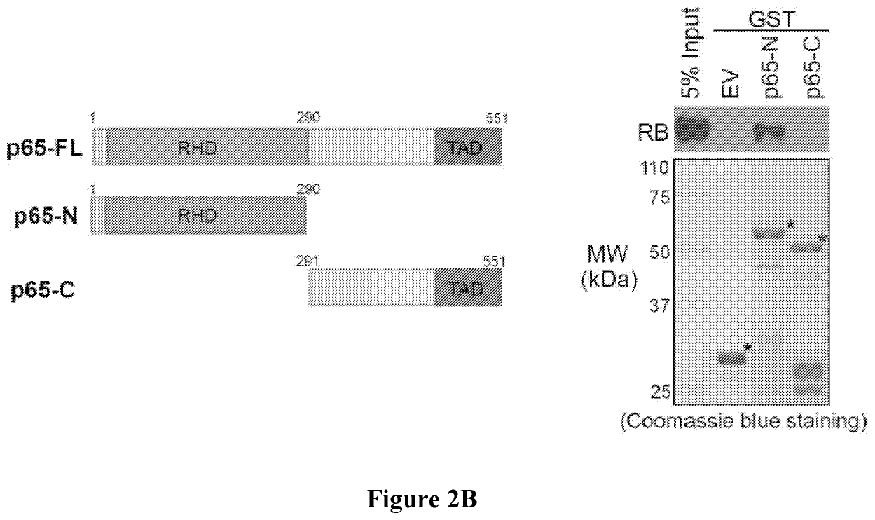

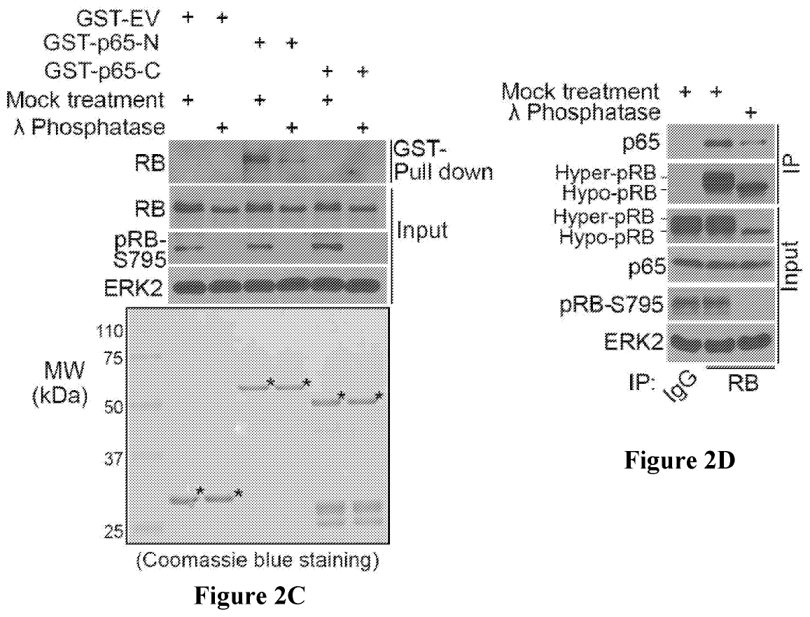

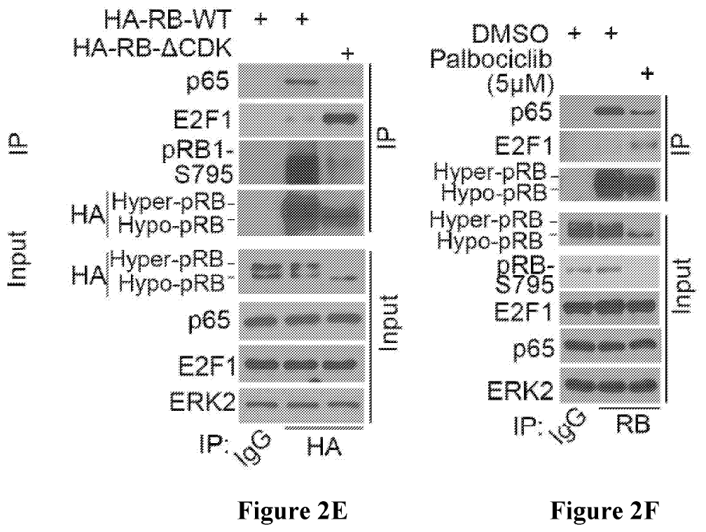

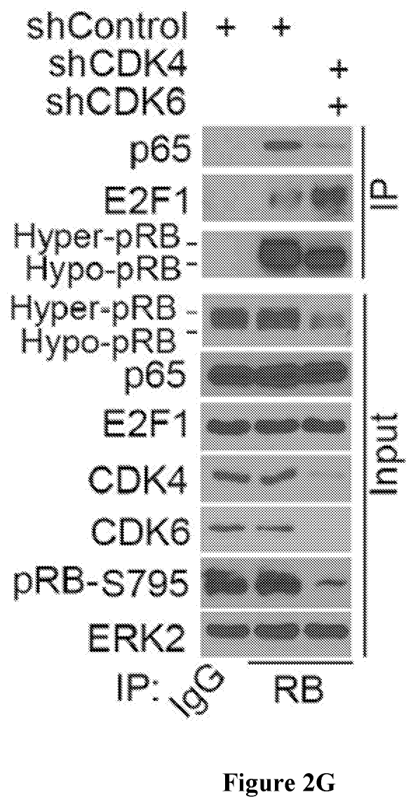

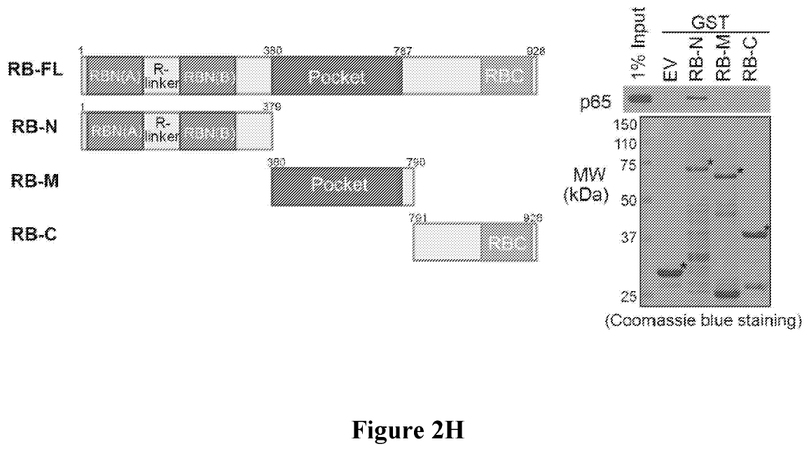

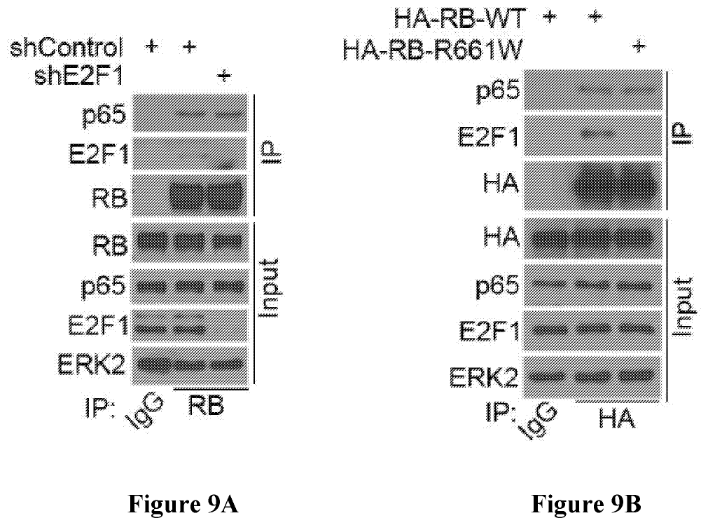

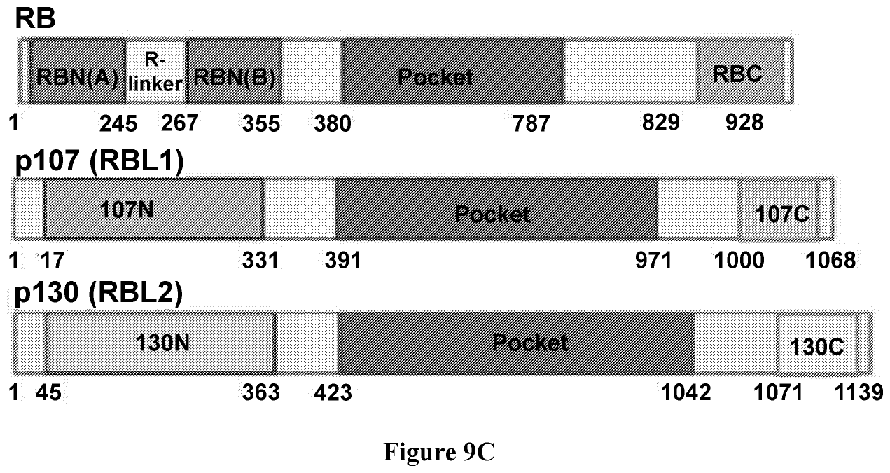

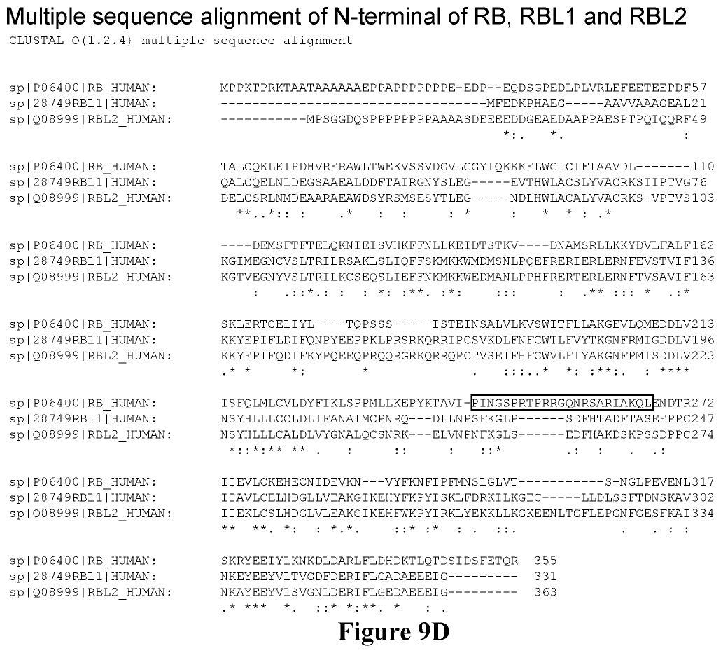

The following was performed to further characterize how NFκB inhibition specifically impacts palbociclib-induced expression of PD-L1. Similar to palbociclib, RB knockdown-induced PD-L1 upregulation also was completely blocked by helenalin (FIGS. 8C and 8D), suggesting that RB acts upstream of NFκB in regulating PD-L1 expression. To test this notion, protein co-immunoprecipitation (co-IP) assay was first performed to determine if RB interacts with NFκB. Co-IP revealed that endogenous RB interacts with endogenous p65, but not p50 subunit of NFκB (FIG. 2A). Reciprocal co-IP revealed that p65 interacts with RB, but not other pocket proteins p107 and p130 (FIG. 2A), suggesting that p65-RB interaction is specific. Glutathione-S-transferase (GST) pull down assay revealed that the RHD motif, the DNA binding domain of p65 (p65-N), but not the COOH-terminal portion specifically bound to the RB protein (FIG. 2B). Given that RB is a highly phosphorylated protein, the following was performed to determine whether p65-RB interaction is regulated by RB protein phosphorylation. PC-3 cell lysate was treated with λ, protein phosphatase before being subjected to GST pull down assay. RB interaction with p65-N was largely reduced by phosphatase treatment (FIG. 2C). The same was true for the interaction between endogenous p65 and RB proteins (FIG. 2D). p65 interaction with the non-phosphorylatable RB mutant RBΔCDK, in which 15 major CDK phosphorylation sites were mutated to alanine residues (Narasimha et al., Elife 3 (2014)), was much weaker compared to wild-type RB (RB-WT) (FIG. 2E). As a positive control, RBΔCDK interaction with E2F1 was much stronger than the WT counterpart (FIG. 2E). In agreement with the finding that palbociclib treatment reduced RB phosphorylation, it largely decreased RB interaction with p65 while increased RB association with E2F1 (FIG. 2F). Similarly, CDK4 and CDK6 co-knockdown decreased RB interaction with p65, but increased RB-E2F1 interaction (FIG. 2G). However, RB-p65 interaction was not affected by E2F1 knockdown or ectopic expression of RB-R661W (R654W in mouse Rb), an E2F1-binding deficient mutant of RB (Sun et al., PNAS, 108:704-709 (2011)) (FIGS. 9A and 9B). These results indicated that unphosphorylatable RB has a minimal level interaction with p65 NFκB protein, but the interaction can be largely augmented by CDK4/6 phosphorylation of RB. In support of this conclusion, GST pull down assay revealed that GST-RB-N, but not GST-RB-C purified from bacteria readily interacted with p65 although the interaction band was much weaker than 1% input (FIG. 2H). Given that the p65-binding region in RB-N is not conserved in p107 and p130 (FIGS. 9C and 9D) and this region is away from the pocket domain that binds to E2F1 (FIG. 2H), these results provide a plausible explanation as to why p65 does not interact with p107 and p130, and p65-RB interaction is not affected by E2F1.

Serine-249 and Threonine-252 (S249/T252) Phosphorylation of RB and 161FQVTV165 Motif (SEQ ID NO: 258) in p65 are Required for p65-RB Interaction

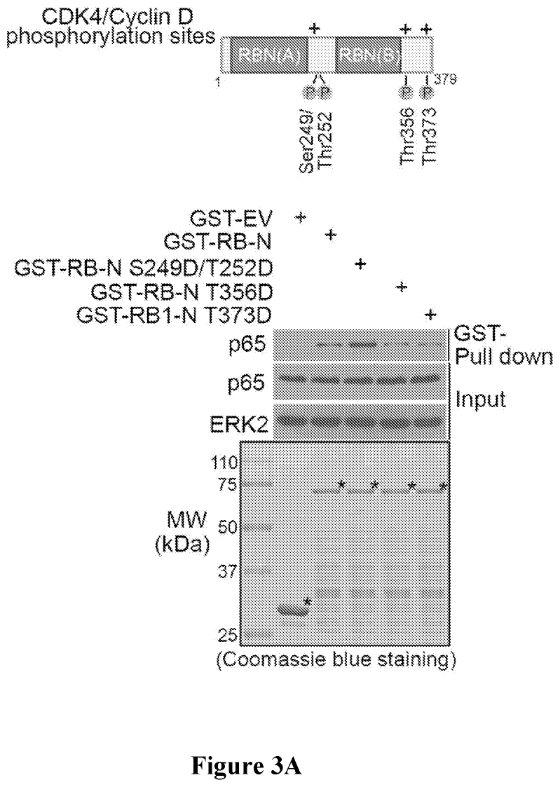

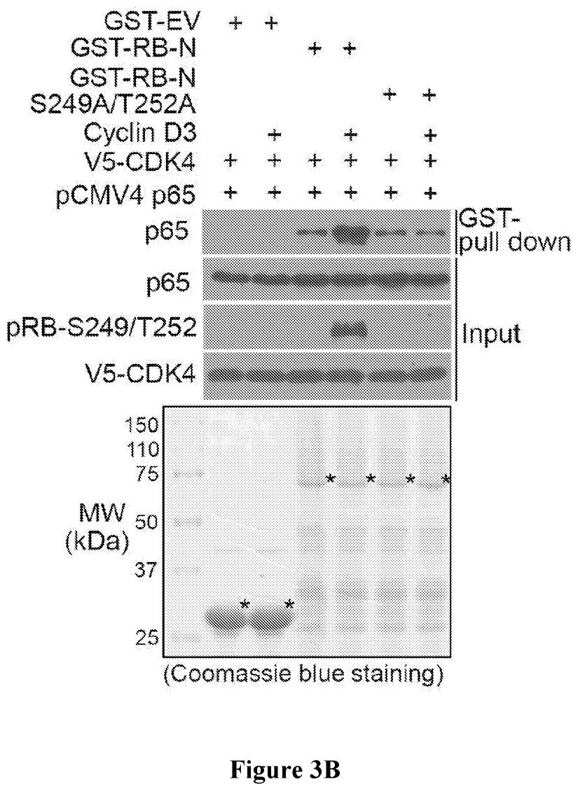

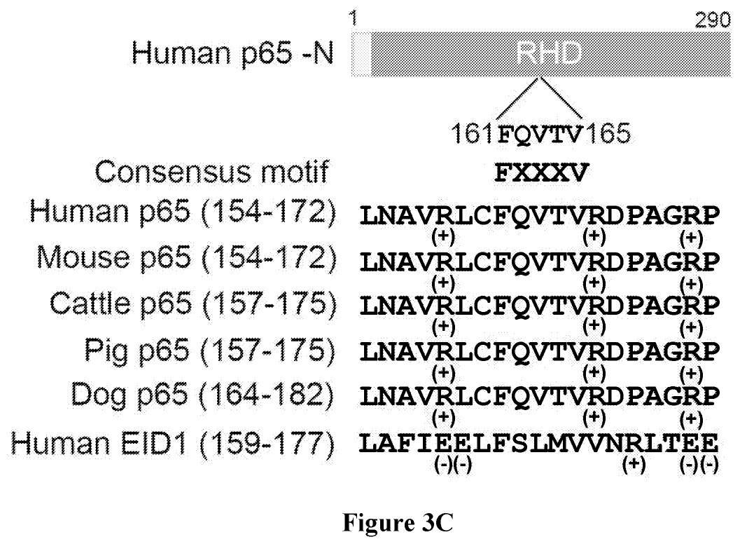

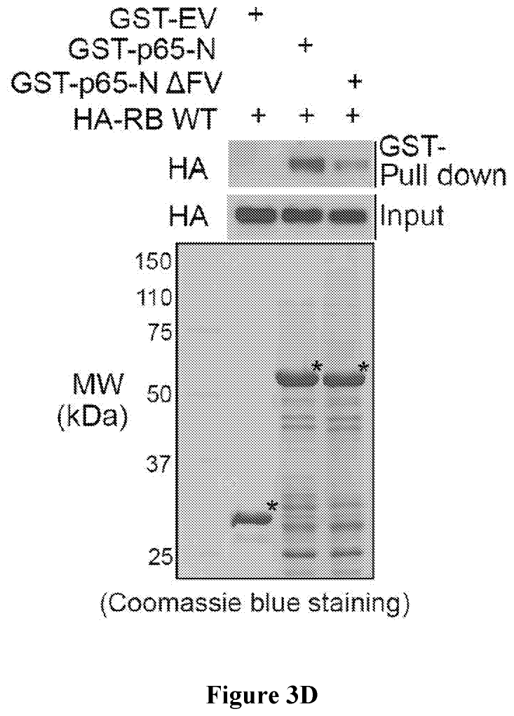

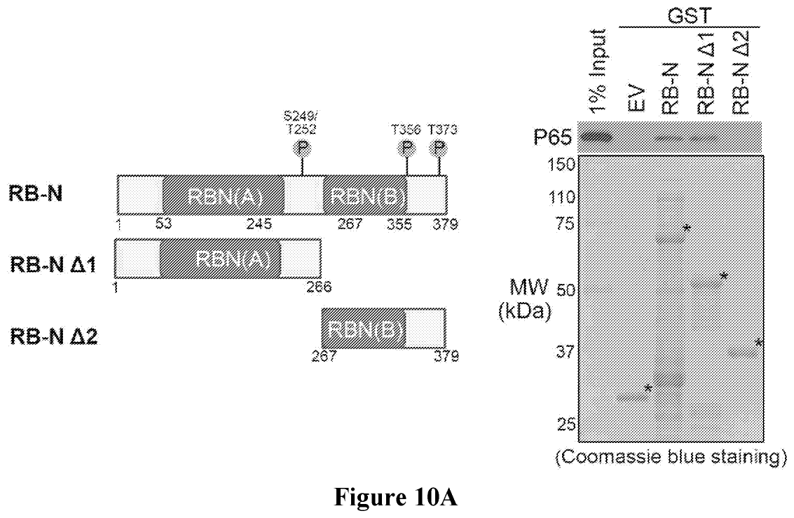

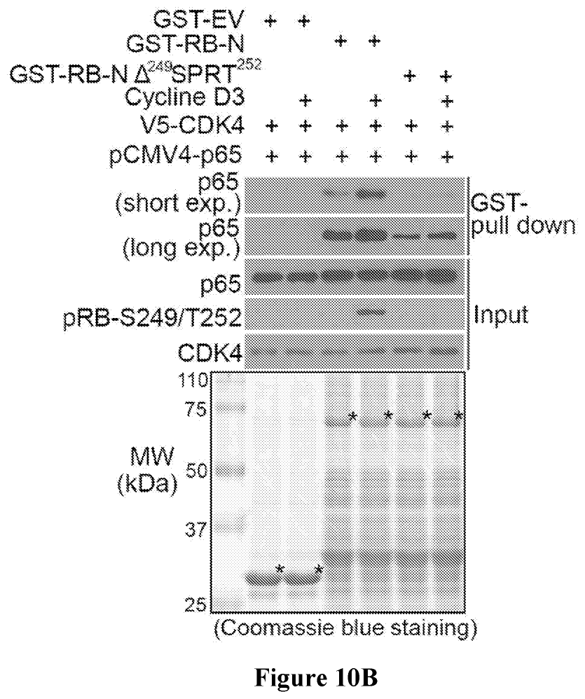

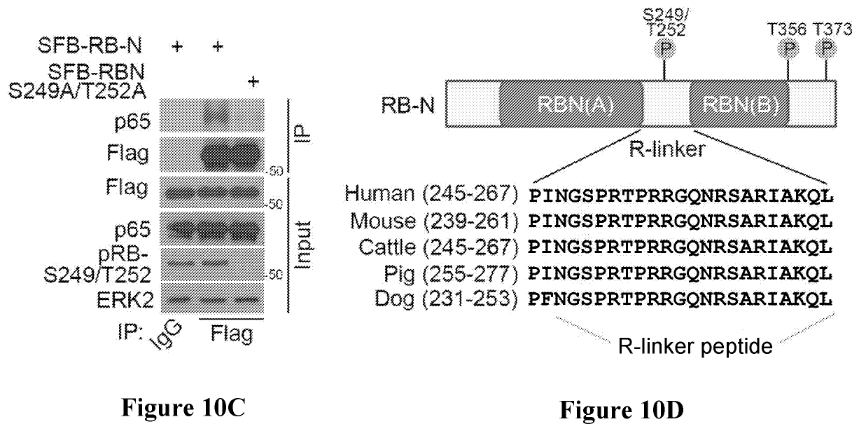

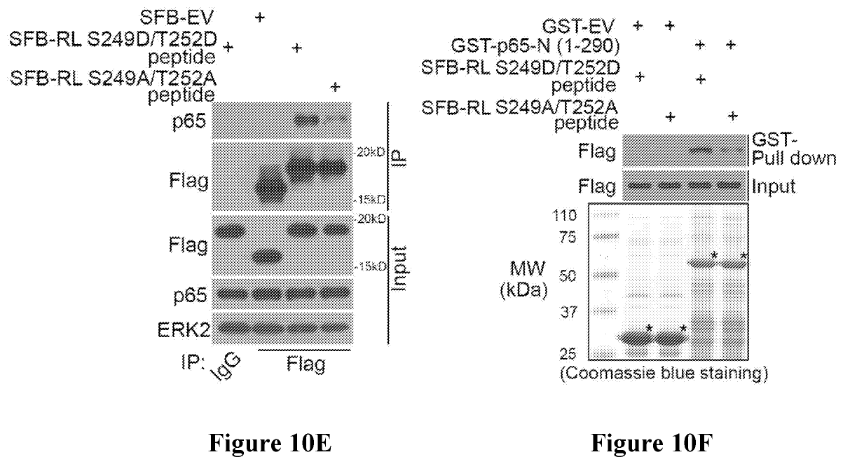

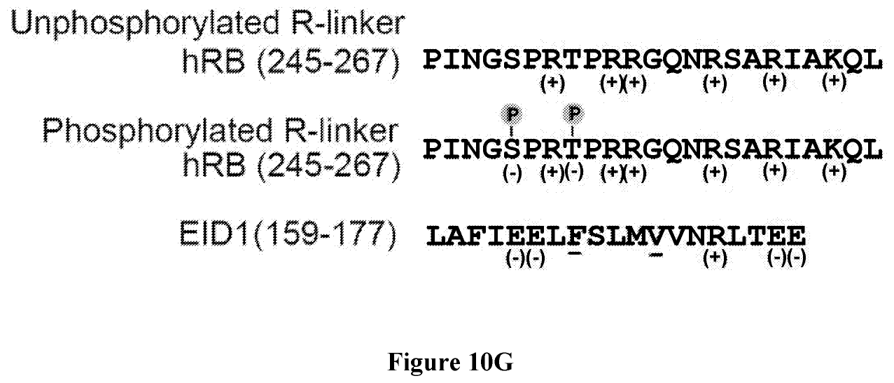

There are four major CDK4 phosphorylation sites present in RB-N, two in the arginine-rich linker (R-linker) region and another two in the C-terminus (Zarkowska et al., J. Biol. Chem., 272:12738-12746 (1997)) (FIG. 3A). Experiments were performed to determine which CDK4 phosphorylation site(s) in RB-N are important for p65 binding. To this end, S249/T252, threonine 356 (T356), or threonine 373 (T373) was mutated separately into aspartic acid (D), an amino acid closely mimicking phosphorylated moiety. A GST pull down assay revealed that only the S249D/T252D mutant exhibited increased interaction with p65 in comparison to unmutated RB-N (FIG. 3A). In agreement with this observation, an in vitro protein binding assay using in vitro transcribed and translated p65 revealed that the N-terminal portion of RB-N (amino acids 1-266 containing S249/T252), but not the C-terminal part (amino acids 267-379 containing T356 and T373) specifically bound to p65 (FIG. 10A). Moreover, an in vitro kinase assay was performed first by inoculating unmutated GST-RB-N or S249/T252 phosphorylation-resistant mutant (S249A/T252A) with the reconstituted CYCLIN D1/CDK4 complex and then an in vitro protein binding assay was performed. S249/T252 phosphorylation on RB-N by CYCLIN D1/CDK4 substantially increased p65-RB-N interaction (FIG. 3B). However, this effect was completely abolished by S249A/T252A mutation and similar results were obtained by deletion of the S249/T252 motif (FIGS. 3B and 10B). By constructing a mammalian expression vector for RB-N with S, Flag, and streptavidin-binding (SFB) tags, it was demonstrated that RB-N strongly bound to p65 in cultured cells and the binding was substantially diminished by S249A/T252A mutation (FIG. 10C). These data indicate that CDK4 phosphorylation of S249/T252 in the R-linker region largely enhances RB-N interaction with p65. It is worth noting that the R-linker and S249/T252 phosphorylation sites are not conserved in RB homologs p107 and p130 (FIGS. S2D and S2E), providing further supporting of the co-IP data (FIG. 2A).

It was determined that the amino acid sequence in the R-linker region that binds to p65 is evolutionally conserved from human to mouse (FIG. 10D). To assess the functional importance of the p65 binding region in RB-N, an SFB-tagged expression vector for a fragment of RB-N containing 21 amino acids surrounding S249/T252, which is termed as R-Linker or RL peptide (FIG. 10D), was constructed. Co-IP assays revealed that p65 was effectively immunoprecipitated by SFB-tagged S249/T252 phospho-mimicking RL peptide (RL-S249D/T252D), but not SFB alone, and such effect was largely diminished by unphosphorylable mutant RL-S249A/T252A (FIG. 10E). In vitro protein binding assays demonstrated that the binding between in vitro transcribed and translated phospho-mimicking RL-S249D/T252D peptide and p65-N was much stronger compared to phosphorylation-resistant mutant S249/T252A (FIG. 10F). Thus, a small phosphor-mimicking peptide from RB-N that binds strongly with p65 was identified.

An FxxxV (166FSLMV170) motif (SEQ ID NO: 286)-centered region in E1A-like inhibitor of differentiation-1 (EID1) is involved for its interaction with RB-N and S249/T252 phosphorylation by CDKs in the R-linker region abrogates in RB-N interaction with E1D1 (Hassler et al., Molecular Cell, 28:371-385 (2007)). It was determined that there are several acidic amino acids (negative charge) in the 166FSLMV170 (SEQ ID NO: 286)-centered region of EID1 whereas the R-linker in RB-N is an arginine (positive charge)-rich region (FIG. 10G). Introduction of negative charges by S249/T252 phosphorylation in the R-linker region impaired RB-N interaction with the negatively-charged FxxxV-centered region in EID1 (FIG. 10G).

Different from an acidic FxxxV-containing region in EID1, an evolutionally conserved 161FQVTV165 (SEQ ID NO: 258) (FxxxV)-centered motif in the RB-binding region of p65 that contains several basic (positive charge) amino acids (FIG. 3C) was identified. Similar to the FxxxV motif in EID1, deletion of the 161FQVTV165 motif (SEQ ID NO: 258) largely diminished the interaction between p65 and RB (FIG. 3D). Based these findings, a working model was developed wherein introduction of negative charge by S249/T252 phosphorylation allows otherwise fully positively-charged RB-N to bind to positively-charged 161FQVTV165 motif (SEQ ID NO: 258) in p65, a mechanism of action opposite to that between E1D1 and RB-N, thereby providing a mechanistic explanation for the observation that RB-p65 interaction was largely diminished by CDK4/6 inhibition (FIGS. 3E and 10G).

### RB Globally Regulates NFkB Transcriptional Program in Cells

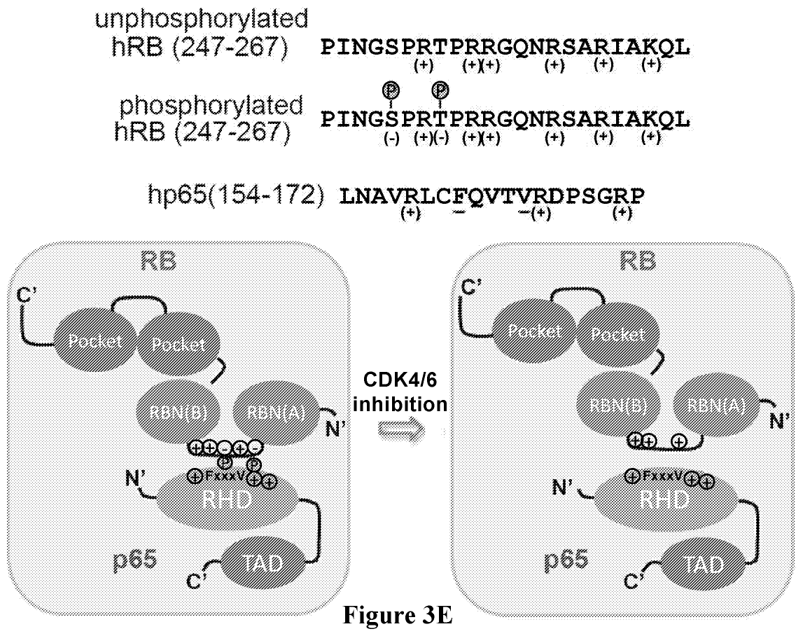

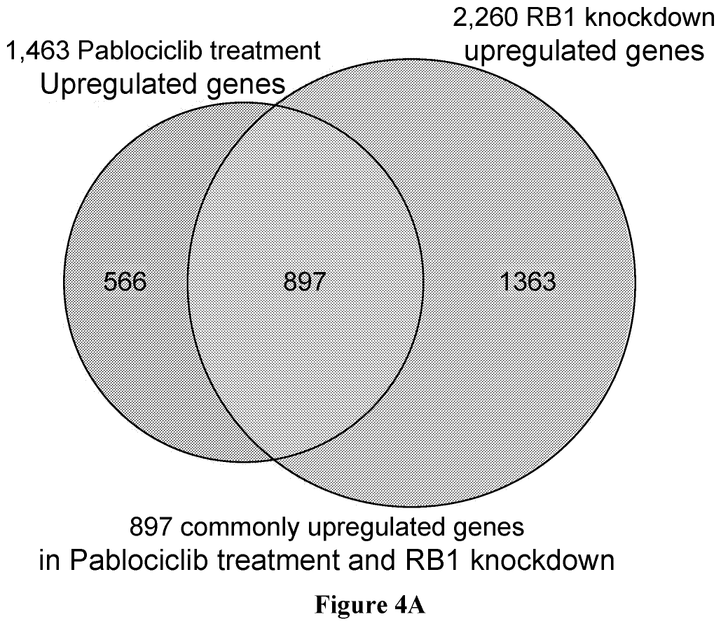

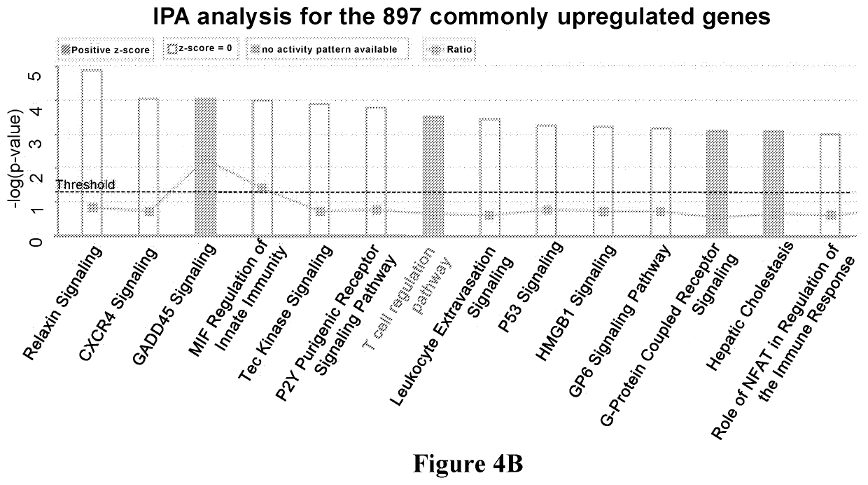

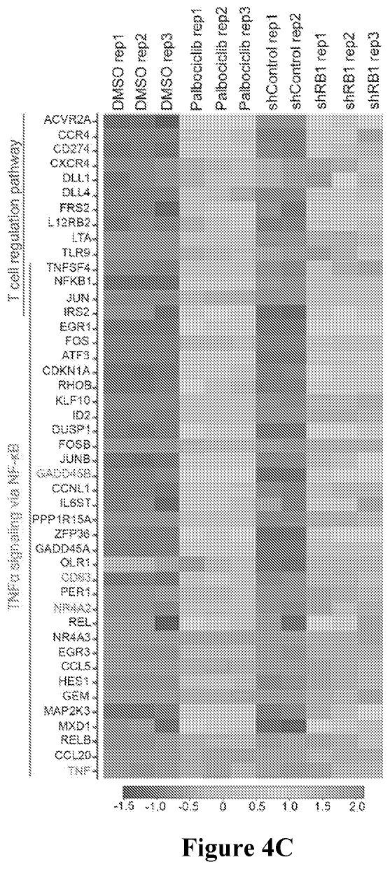

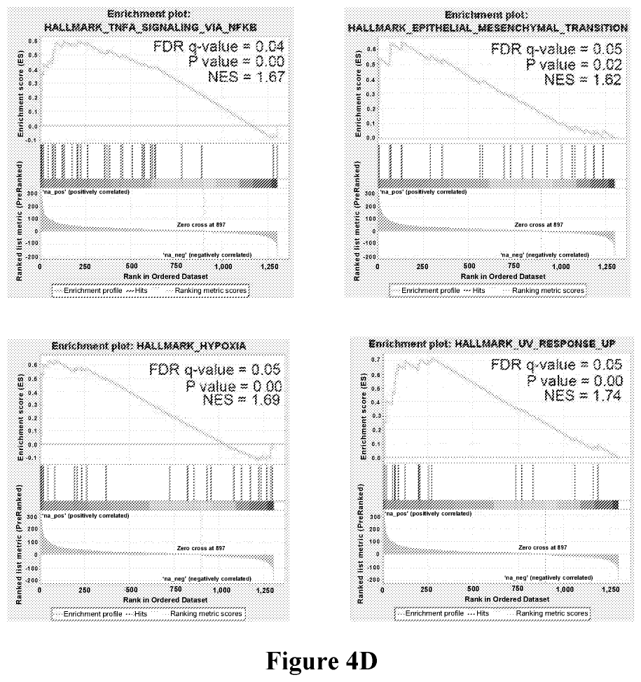

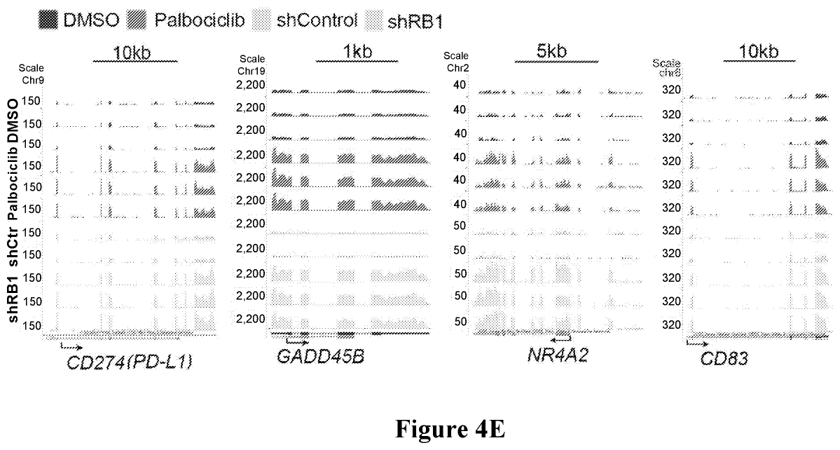

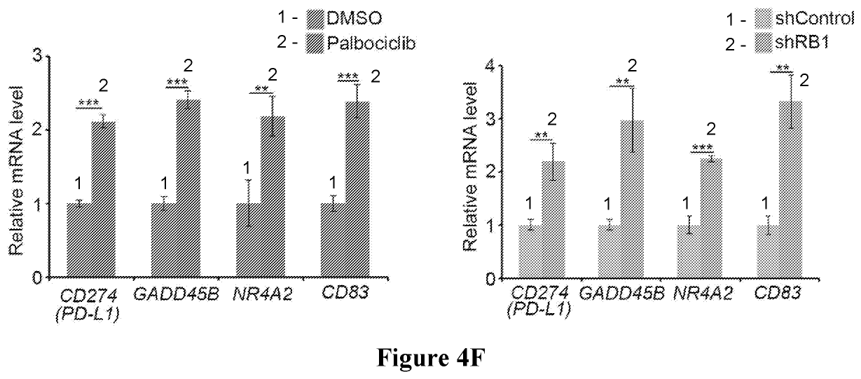

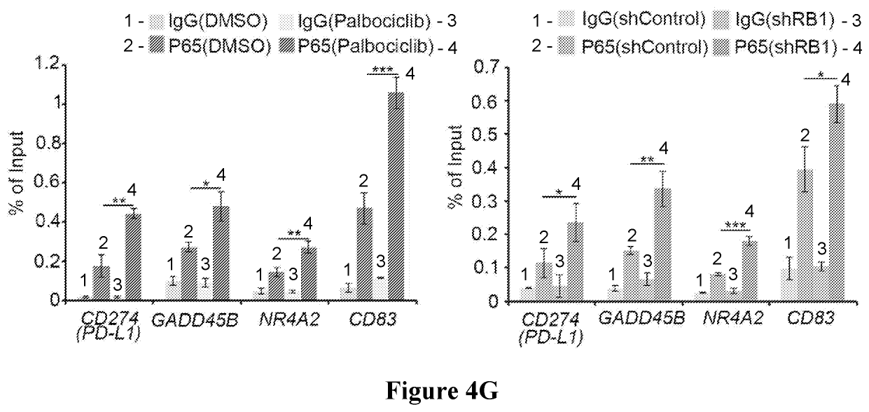

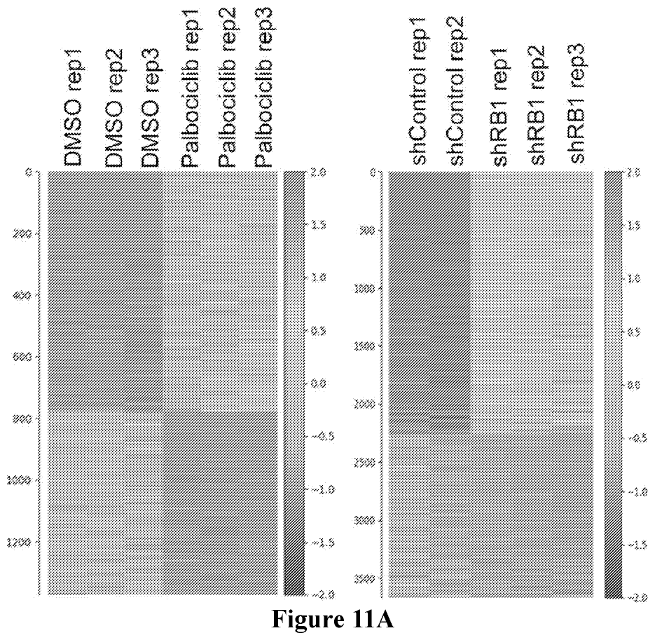

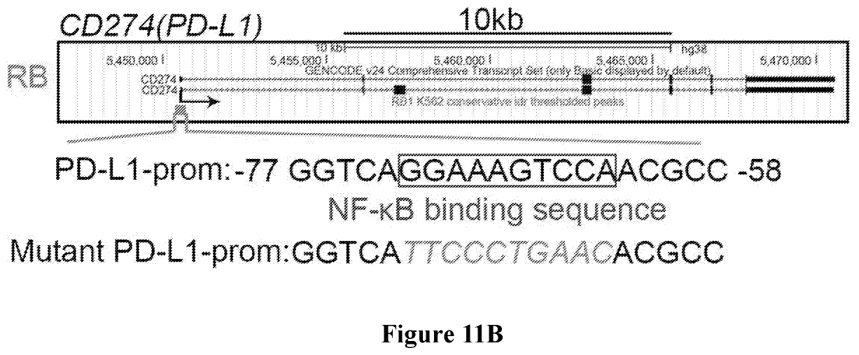

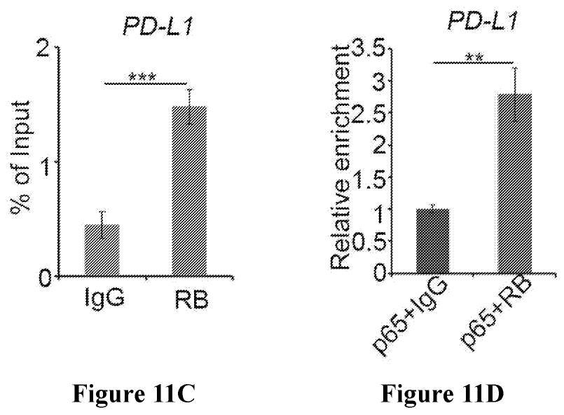

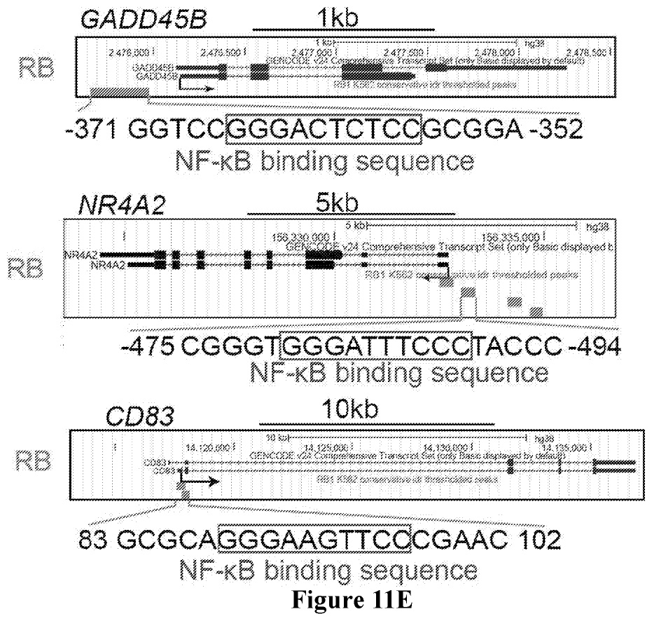

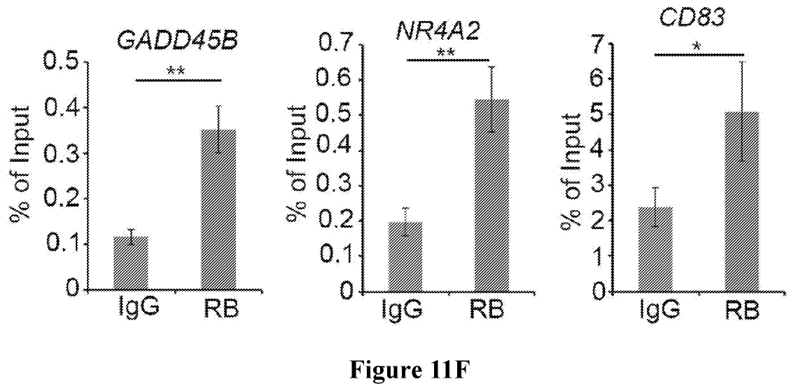

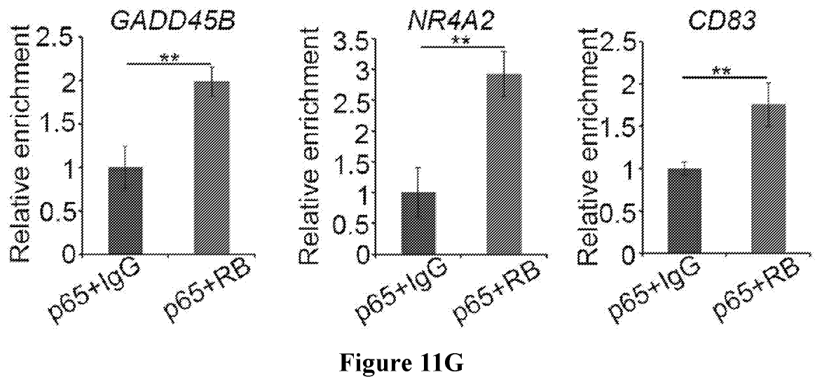

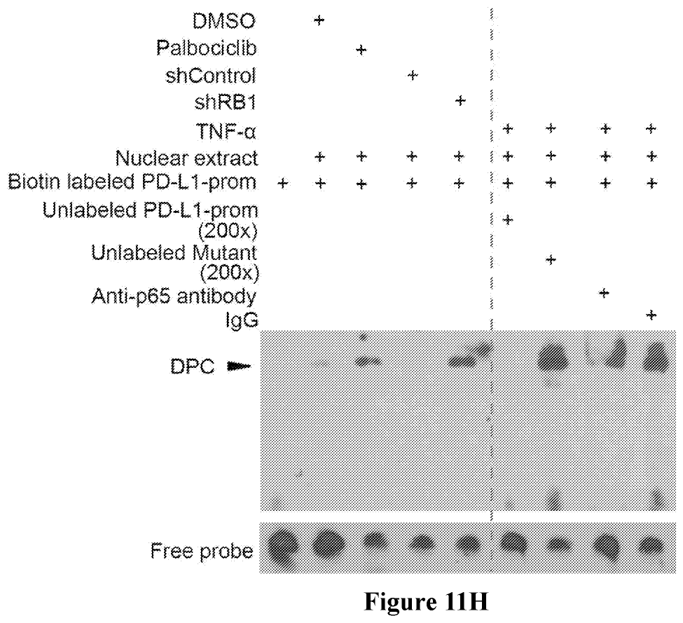

Experiments were performed to determine whether the NFκB transcriptional program is globally regulated by RB phosphorylation. Endogenous RB was knocked down in PC-3 cells or cells were treated with the CDK4/6 inhibitor palbociclib, and RNA was isolated for high throughput sequencing (RNA-seq). A large set of genes was identified whose expression was invariably downregulated or upregulated by both RB knockdown and palbociclib among three replicates (one inconsistent replicate from control knockdown cells was removed for further analysis) (FIGS. 4A and 11A). Ingenuity pathway analysis (IPA) of 897 commonly upregulated genes revealed that T cell regulatory pathway genes including CD274 (PD-L1) were among the signaling pathways significantly affected by RB knockdown and palbociclib treatment (FIGS. 4A-4C). Gene set enrichment analysis (GSEA) revealed that the upregulated genes significantly overlapped with hallmark TNFα-NFκB pathway targets and epithelial-to-mesenchymal transition (EMT), hypoxia, and ultraviolet (UV) response genes (FIGS. 4C and 4D). Expression of the genes commonly upregulated by palbociclib and RB knockdown, including PD-L1 and other NFκB targets GADD45B, NR4A2, and CD83, were exemplified in FIG. 4E, and elevation of them was further confirmed by reverse transcription-quantitative polymerase chain reaction (RT-qPCR) (FIG. 4F). These results indicated that expression of NFκB target genes, including PD-L1, was globally regulated by the CDK4/6-RB pathway.

Chromatin immunoprecipitation coupled quantitative PCR (ChIP-qPCR) analysis revealed that p65 protein readily bound to the genomic loci of the RB affected genes including PD-L1, and p65 binding was substantially increased by palbociclib treatment or RB knockdown in PC-3 cells (FIG. 4G). Meta-analysis of published RB ChIP high throughput sequencing (ChIP-seq) data (Consortium, Nature, 489:57-74 (2012)) revealed that there is a RB binding peak in the PD-L1 gene promoter (FIG. 11B). RB binding at this locus was further confirmed by RB ChIP and p65 ChIP/RB re-ChIP assays (FIGS. 11C and 11D). Similar results were obtained in the genomic loci of other NFκB target genes analyzed (FIGS. 11E-11G). Moreover, there was a putative NFκB DNA binding sequence (NBS) in the RB binding peak region in the promoter of PD-L1 and other NKκB target genes examined (FIGS. 11B and 11E). Electrophoretic mobility shift assays (EMSA) were performed using a biotin-labeled NBS-containing 20-nucleotide DNA sequence (PD-L1-Prom) as probe. A DNA-protein complex (DPC) was detected in nuclear extract of PC-3 cells treated with vehicle or control shRNA, and the DPC level was largely augmented by palbociclib treatment or RB knockdown (FIG. 11H). In cells treated with TNFα, the DNA-protein binding was largely enhanced, and the binding was completely blocked by excessive amount of unlabeled wild-type probe, but not the NBS-mutated counterpart (FIG. 11H). Also, the binding signal was reduced by inoculation with p65-specific antibody, and no such effect was detected for non-specific IgG (FIG. 11H). Thus, a functional NBS site was identified in the PD-L1 promoter, binding of which by p65 is regulated by the CDK4/6-RB signaling cascade.

### Involvement of S249/T252 Phosphorylation of RB in Regulation of PD-L1 Expression

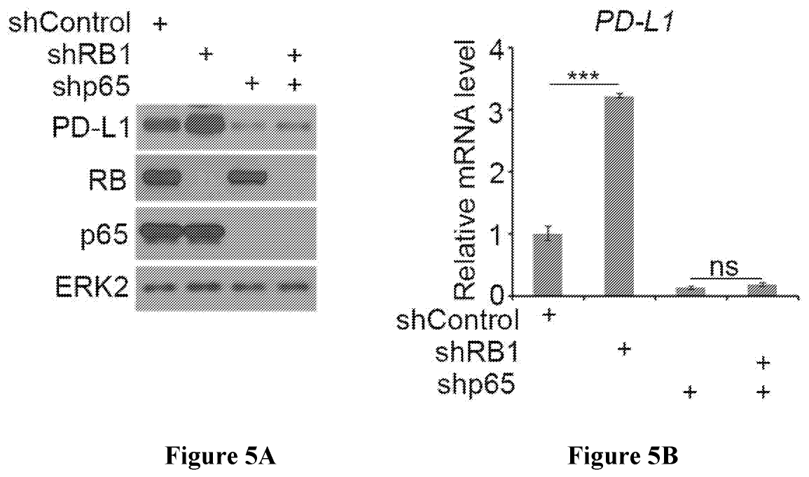

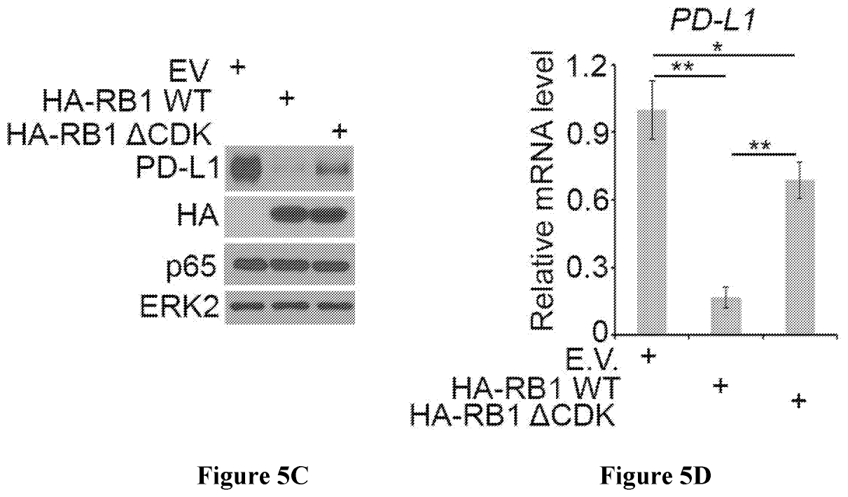

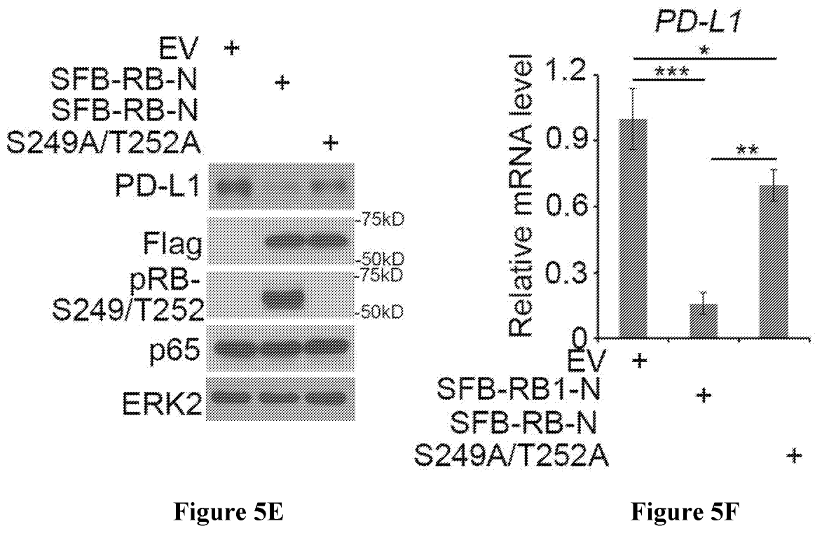

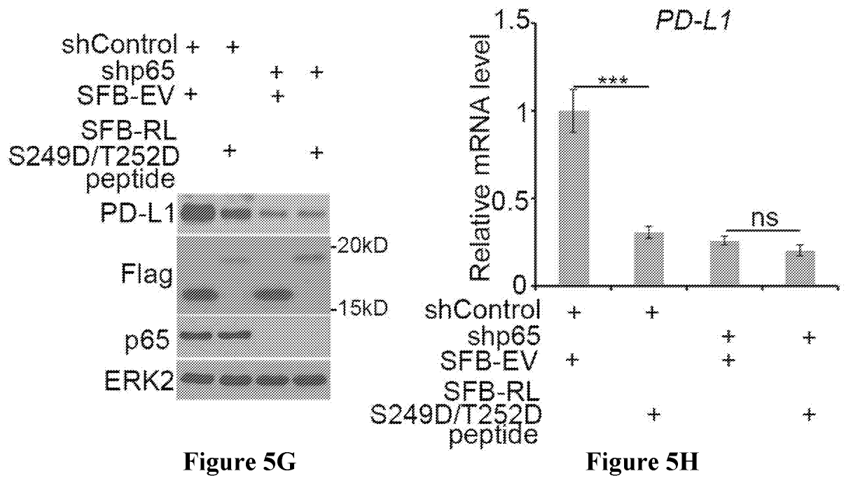

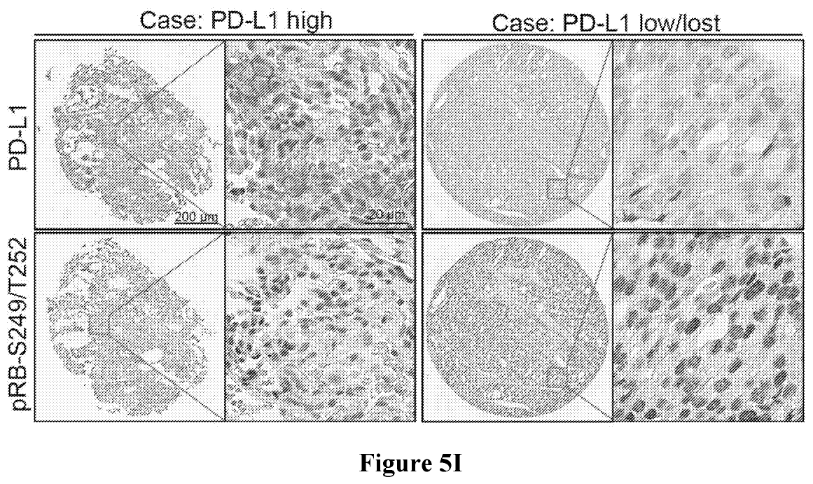

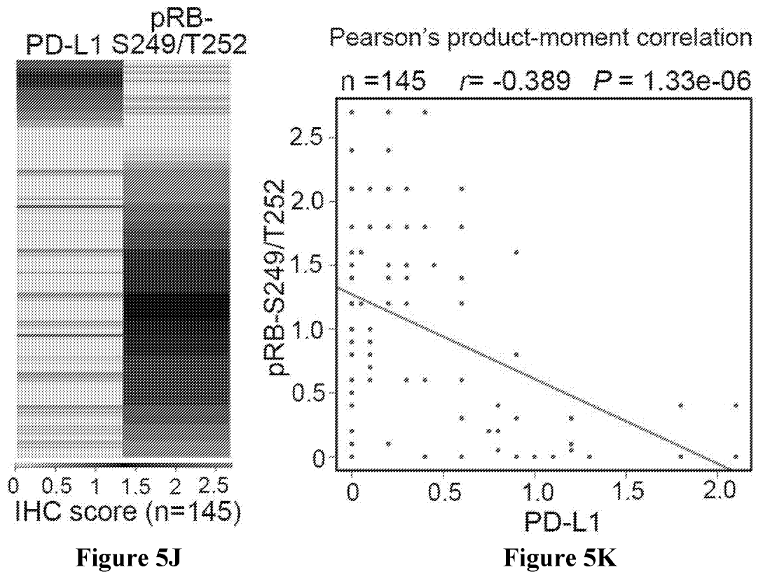

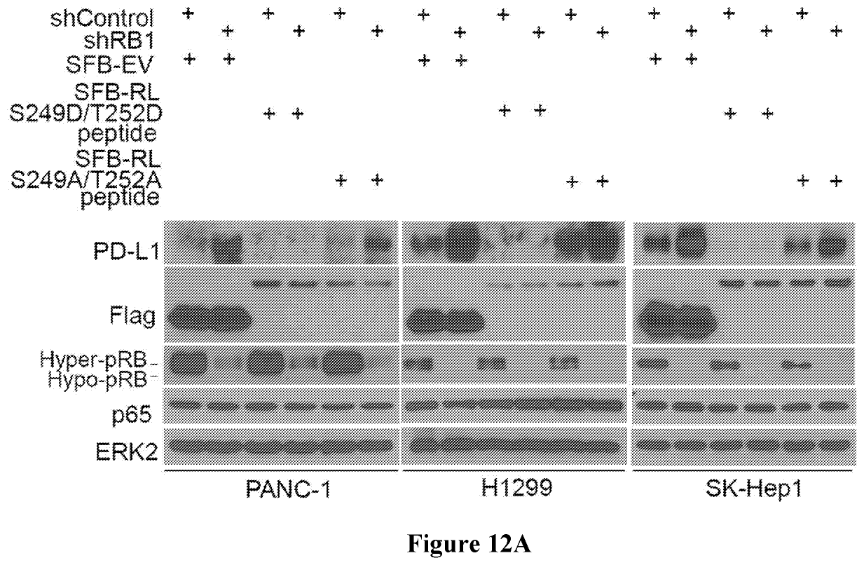

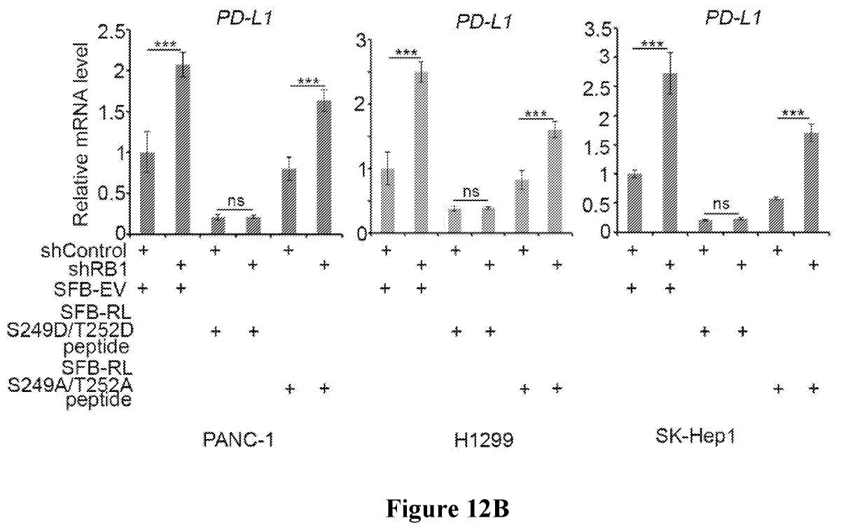

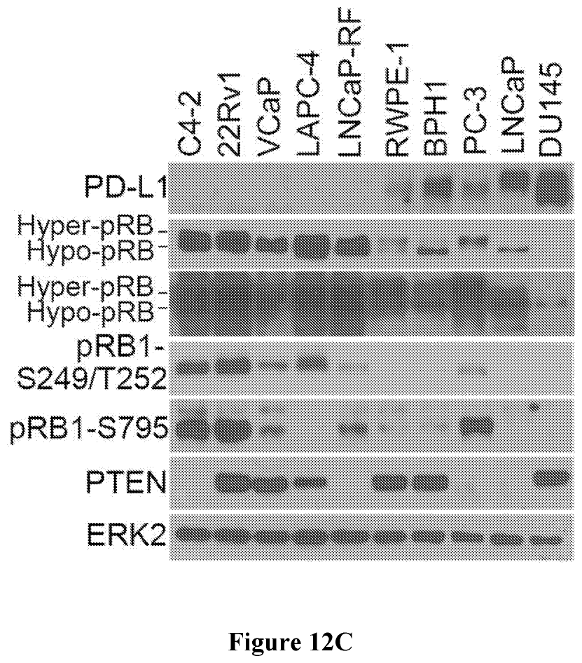

In keeping with the finding that RB knockdown-induced PD-L1 expression was inhibited by the NFκB inhibitor helenalin (FIGS. 8D and 8E), upregulation of PD-L1 mRNA and protein due to RB knockdown was abrogated by co-knockdown of p65 in PC-3 cells (FIGS. 5A and 5B). Ectopic expression of RB WT substantially decreased PD-L1 expression, and this effect was largely attenuated in cells expressing the phosphorylation-resistant mutant RBΔCDK (FIGS. 5C and 5D). It is worth noting that PD-L1 expression level was lower in RBΔCDK-expressing cells compared to control cells (FIGS. 5C and 5D). This was probably due to the presence of the basal/minimal level interaction between the phosphorylation-resistant mutant of RB and p65 (FIG. 2E). Expression of RB-N, the p65-binding region of RB was sufficient to inhibit PD-L1 at both mRNA and protein levels, but this effect was largely diminished by S249A/T252A mutant (FIGS. 5E and 5F). Consistent with the protein binding results, expression of the small phospho-mimicking peptide derived from the p65-binding motif in RB-N(SFB-RL-peptide-S249D/T252D) largely inhibited PD-L1 expression in p65-proficient, but not in p65-depleted PC-3 cells (FIGS. 5G and 5H). Similar results were obtained in cell lines of several other cancer types (FIGS. 12A and 12B), indicating that this peptide is bioactive in a broad spectrum of cancer types. These results demonstrate that a small phospho-mimicking peptide of RB-N can be used to inhibit PD-L1 expression in a manner dependent on p65.

Consistent with the finding in cultured cancer cells that S249/T252 phosphorylation is involved in RB binding of p65 and RB-mediated repression of PD-L1 expression, RB phosphorylation, especially S249/T252, but not total RB protein, was generally corrected with PD-L1 protein expression in an array of prostate cancerous and noncancerous cell lines (FIG. 12C). Experiments were performed to determine whether there was a connection between S249/T252-phosphorylated RB (pRB-S249/T252) and PD-L1 expression in patient samples. Immunohistochemistry (IHC) was performed on a tissue microarray (TMA) of a cohort of metastatic castration-resistant prostate cancer (mCRPC) specimens (n=145 TMA elements) using PD-L1 and RB S249/T252 phosphorylation-specific antibodies. IHC staining was evaluated by measuring both staining intensity and percentage of positive cells. Representative IHC images showing high and low/lost staining of PD-L1 and pRB-S249/T252 were shown in FIG. 51. In this cohort, 65 out of 145 (44.8%) TMA specimens were PD-L1 positive, but only 17 out of 145 (11.7%) TMA specimens expressed moderate to high level of PD-L1 protein in cell surface, suggesting that the overall rate of PD-L1 expression was low in prostate cancer specimens, which was consistent with the provided elsewhere (Gevensleben et al., Clinical Cancer Research, 22:1969-1977 (2016); and Massari et al., Target Oncology, 11:345-351 (2016)). Further analysis revealed that PD-L1 protein expression inversely correlated with pRB-S249/T252 level in this cohort (Pearson's product-moment correlation r=−0.389, P=1.33e−06) (FIGS. 5J and 5K). These data suggested that regulation of PD-L1 expression by S249/T252-phosphorylated RB also may occur in mCRPC patient specimens.

### Small S249/T252 Phosphorylation-Mimicking Peptide of RB Blocks Irradiation-Induced PD-L1 Expression and Inhibits Cancer Immune Evasion

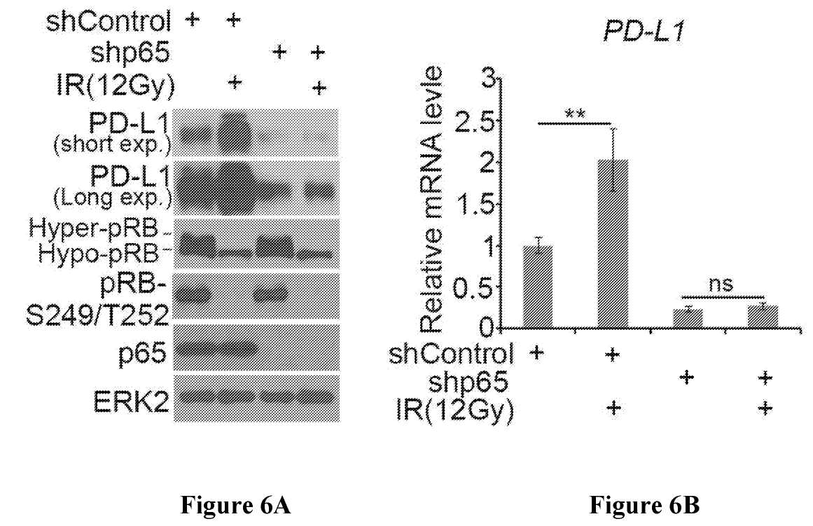

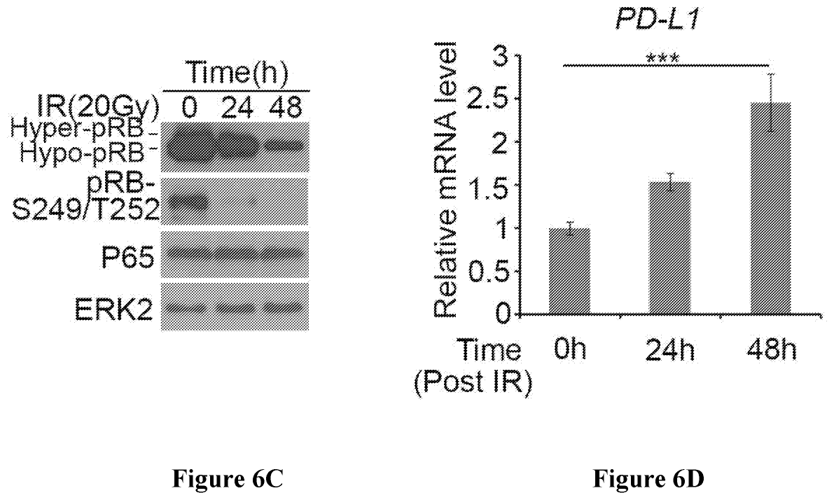

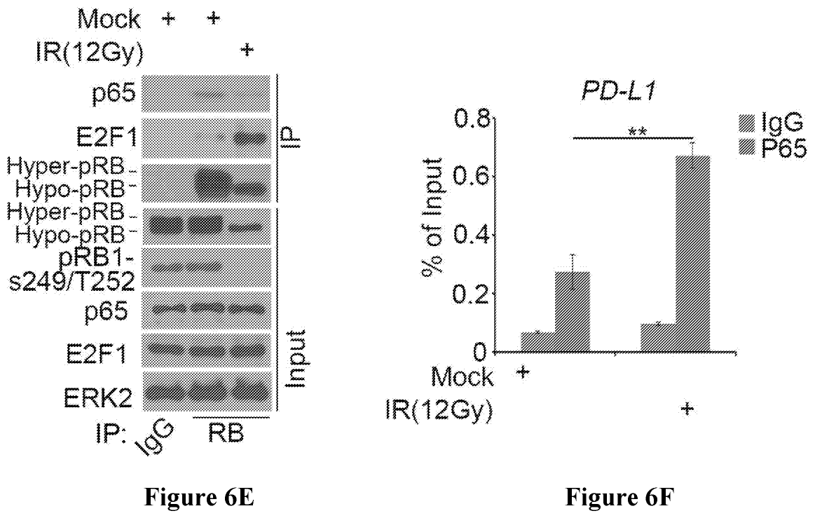

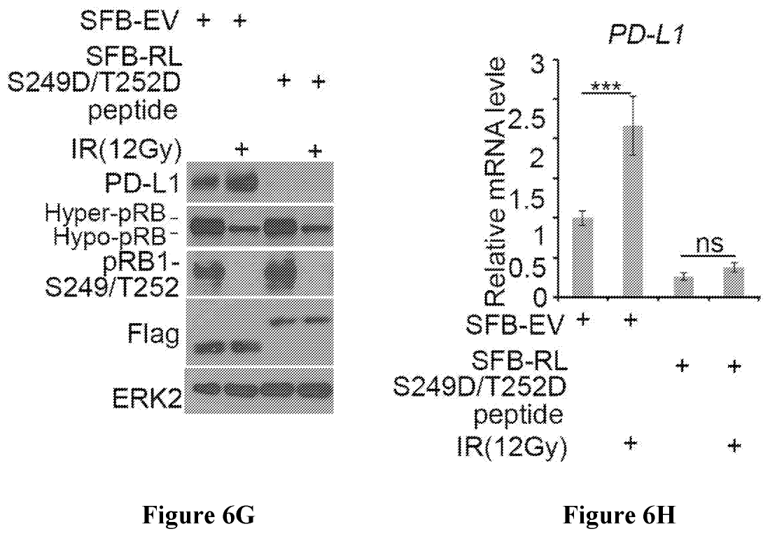

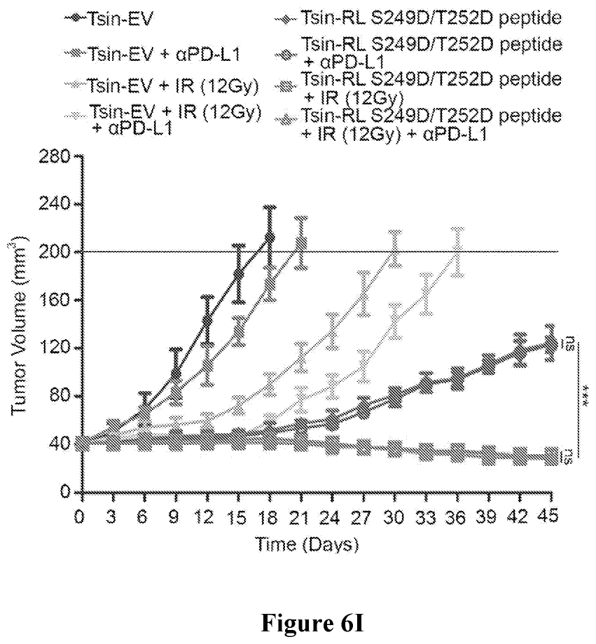

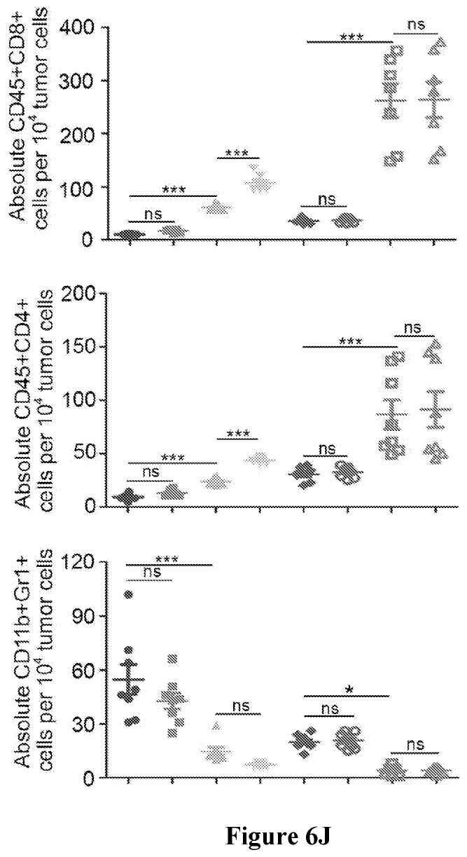

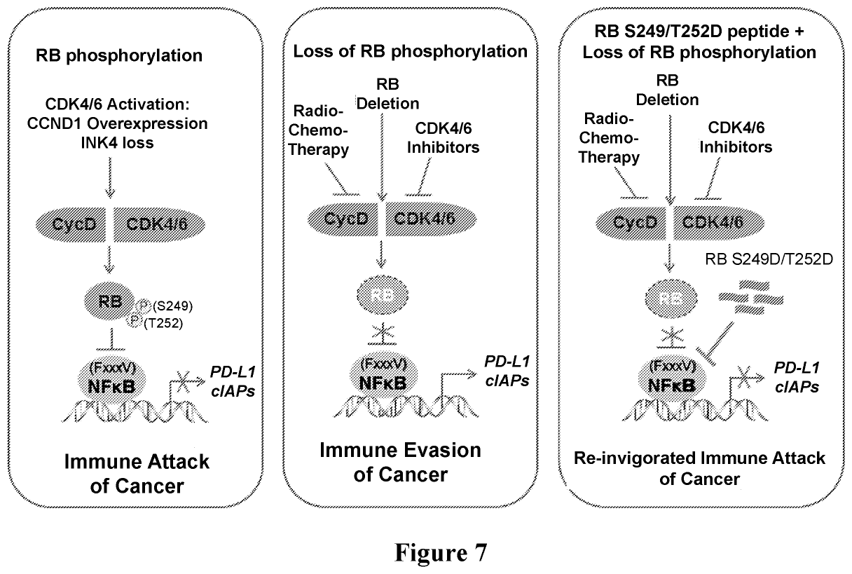

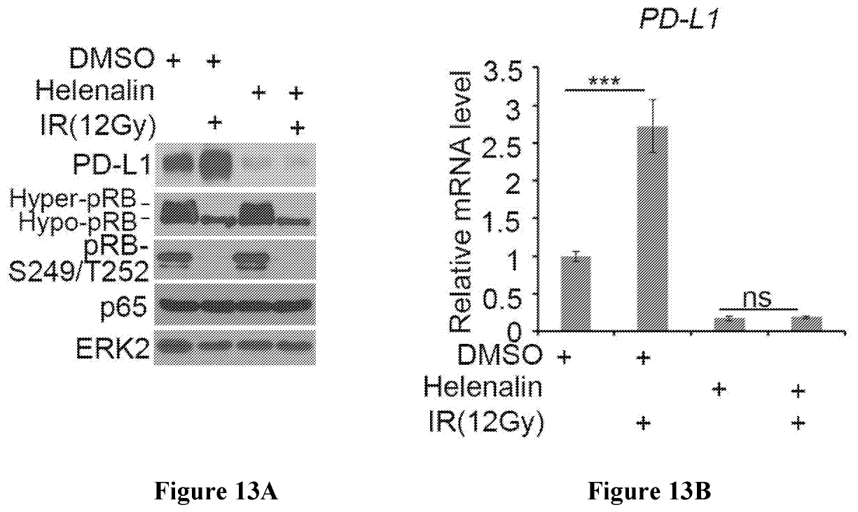

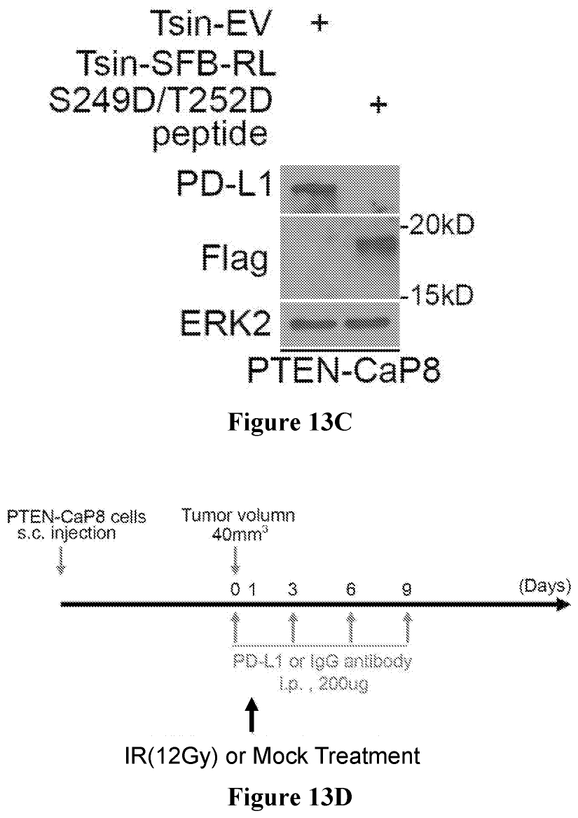

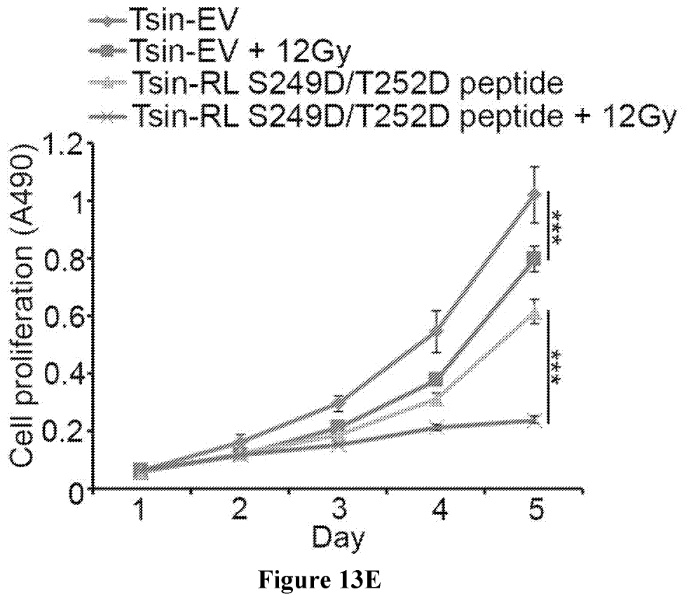

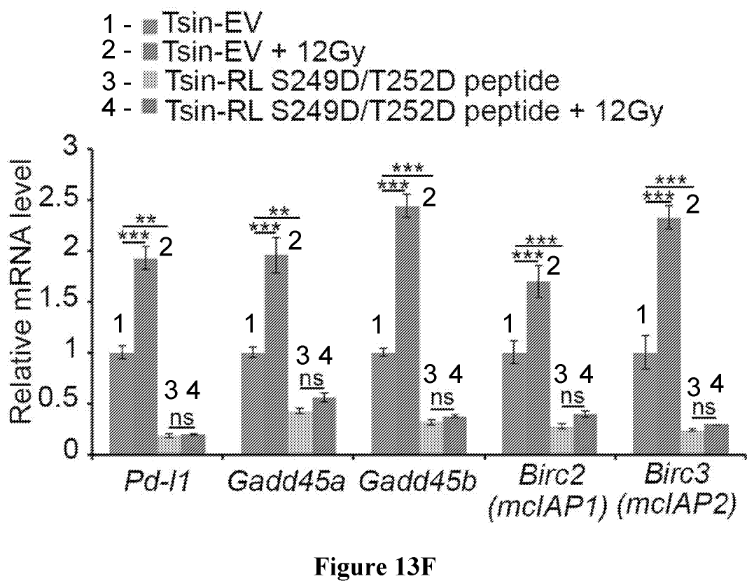

Irradiation can cause cell cycle arrest and downregulation of RB phosphorylation (Abraham, Genes Dev., 15:2177-2196 (2001)). Based upon the data presented herein, it was hypothesize that PD-L1 expression can be induced by irradiation, and this effect is likely reversed by treatment of S249/T252 phosphorylation-mimicking peptide. Gamma irradiation inhibited RB phosphorylation at S249/T252 and markedly increased PD-L1 mRNA and protein expression in a time-dependent manner in PC-3 cells (FIGS. 6A-6D, 13A, and 13B). The effect of gamma irradiation was abrogated by shRNA-mediated knockdown of p65 or treatment of the NFκB inhibitor helenalin (FIGS. 6A, 6B, 13A, and 13B). These results were consistent with the observations that gamma irradiation decreased RB phosphorylation at S249/T252 and RB binding with p65, but increased p65 binding at the PD-L1 gene promoter (FIGS. 6E and 6F). Moreover, ectopic expression of the small RB S249/T252-phosphorylation-mimicking peptide RL-S249D/T252D not only decreased PD-L1 basal level, but also largely, but not completely blocked gamma irradiation-induced upregulation of PD-L1, at least in mRNA level (FIGS. 6G and 6H).

Induction of PD-L1 expression by gamma irradiation suggested that immune checkpoint blockade and the bioactive S249/T252-phosphorylation-mimicking peptide (RL-S249D/T252D peptide) of RB might enhance the anti-tumor efficacy of radiotherapy. PTEN-CaP8 mouse prostate tumor-bearing mice were treated with gamma irradiation (12 Gy) or mock treated in combination with anti-Pd-l1 or a non-specific control IgG (FIGS. 13C and 13D). Pd-l1 antibody treatment alone resulted in slight, but not significant inhibition of tumor growth (FIG. 6I). In contrast, gamma irradiation alone substantially decreased tumor growth, and tumor growth was completely blocked by the combination of irradiation and anti-Pd-l1 in the first 15 days of treatment (FIG. 6I). In agreement with the result that the RL-S249D/T252D peptide almost completely blocked PD-L1 expression (FIG. 6G), treatment with the RL-S249D/T252D peptide markedly inhibited tumor growth regardless of Pd-l1 antibody treatment (FIG. 6I). Most importantly, tumors regressed when treated with irradiation plus the small peptide (FIG. 6I). Notably, while inhibiting PD-L1 expression, RL-S249D/T252D peptide treatment also blocked expression of anti-apoptotic NFκB target genes (FIGS. 13E and 13F), providing an explanation as to how this peptide outperforms anti-PD-L1 antibody in tumor suppression. Moreover, irradiation alone, but not Pd-l1 antibody treatment alone, significantly increased tumor infiltration of immune effectors including CD45⁺CD8⁺ T cells and CD45⁺CD4⁺ T cells, but decreased the infiltration of CD11b⁺Gr1⁺ myeloid cells in tumors (FIG. 6J). The combination of irradiation and Pd-l1 antibody additionally increased CD45⁺CD8⁺ and CD45⁺CD4⁺ T cell infiltration in tumors (FIG. 6J). In agreement with the effect on tumor regression, co-treatment of irradiation and the RL-S249D/T252D peptide induced a dramatic increase in CD45⁺CD8⁺ and CD45⁺CD4⁺ T cell infiltration, but a sharp decrease of CD11b⁺Gr1⁺ myeloid cells in tumors (FIG. 6J).

In summary, the results provided herein demonstrate that CDK4/6 inhibitor-induced upregulation of PD-L1 expression occurs at mRNA level, and this effect is RB-dependent. Mechanistically, RB directly interacts with the NFκB protein p65, and the interaction is largely enhanced by CDK4/6 phosphorylation of S249/T252 sites in the N-terminal portion of RB-N. The results provided herein also demonstrate the development of a small RB-derived S249/T252 phospho-mimicking peptide that not only inhibits the basal level of PD-L1, but almost completely blocks irradiation-induced upregulation of PD-L1. This document also identifies a previously uncharacterized tumor suppressor function of phosphorylated RB that suppresses NFκB transcription activity, PD-L1 expression, and tumor immune evasion. Taken together, these results suggest that this activity of RB can be exploited to overcome immune destruction resistance associated with current therapeutics including radio- and chemo-therapy and CDK4/6 small molecule inhibitors in the clinic.

### Example 2—Phosphorylated RB Promotes Cancer Immunity by Inhibiting NFκB Activation and PD-L1 Expression

This example builds on and includes results from Example 1.

### Identification of RB as a Negative Regulator of NFκB Signaling

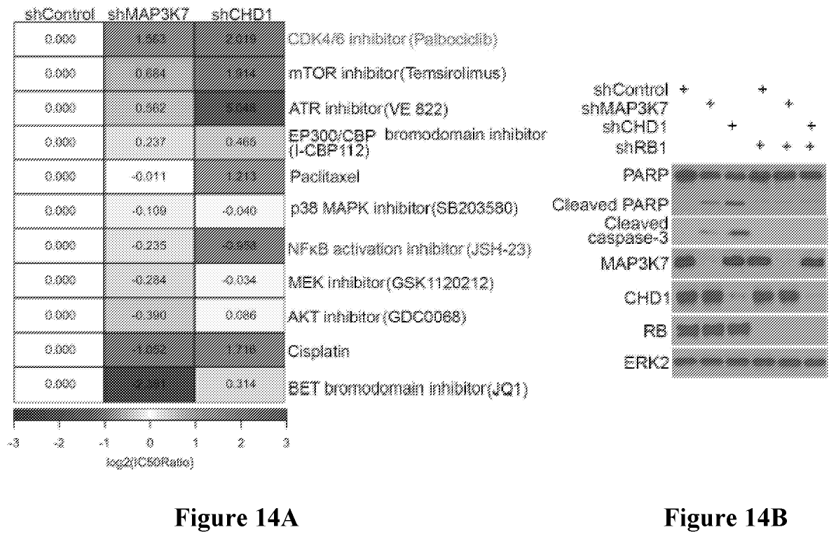

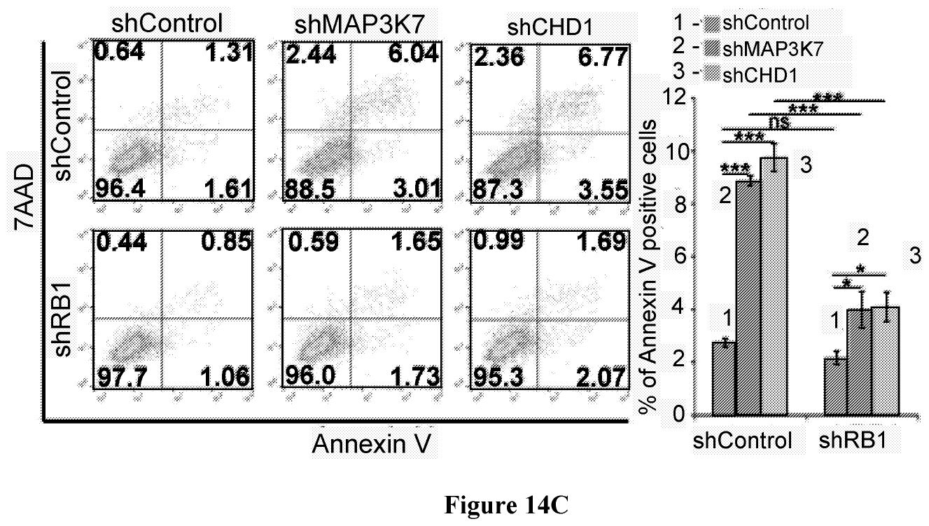

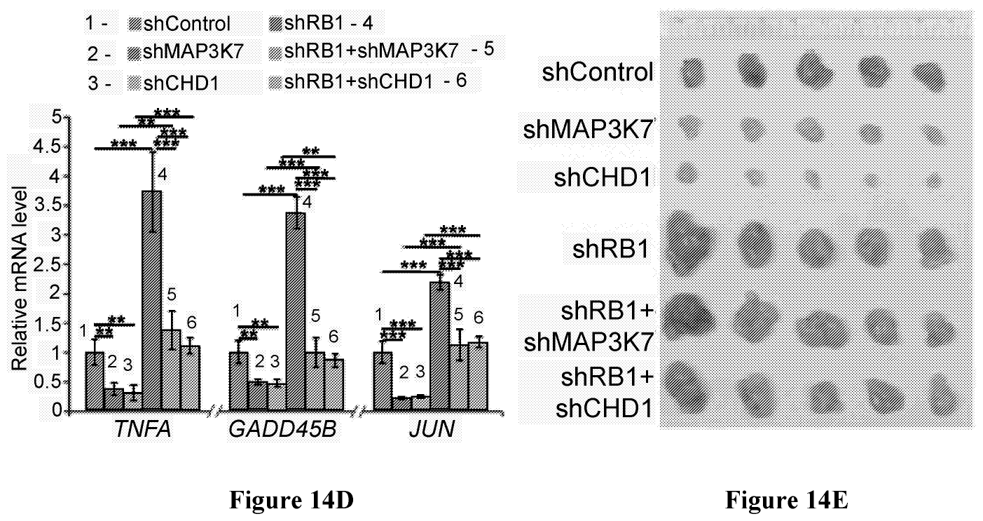

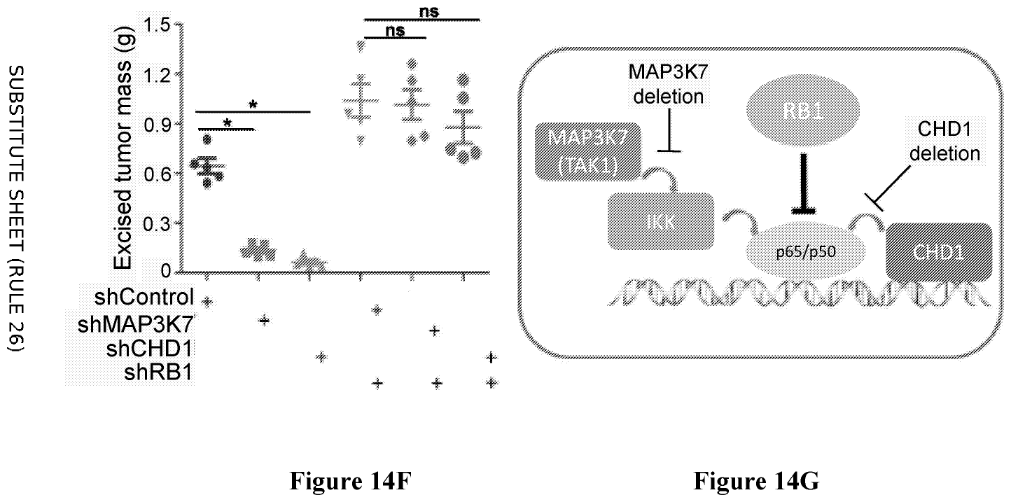

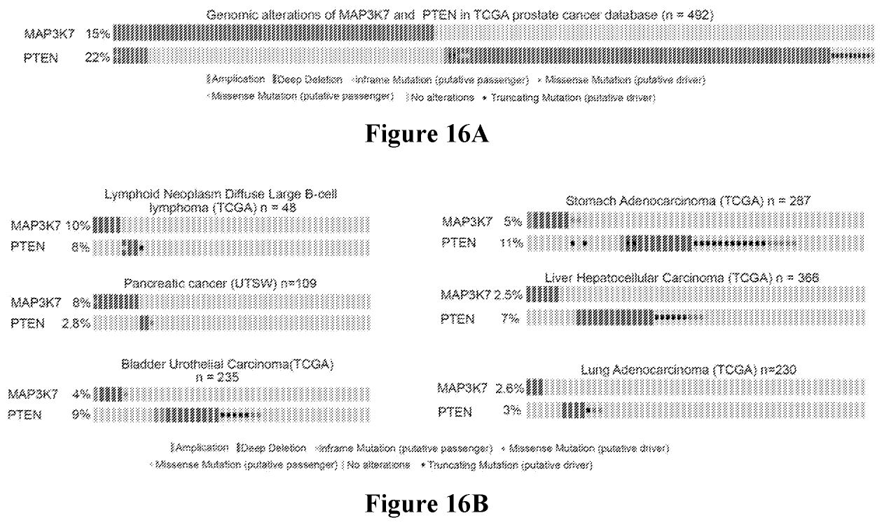

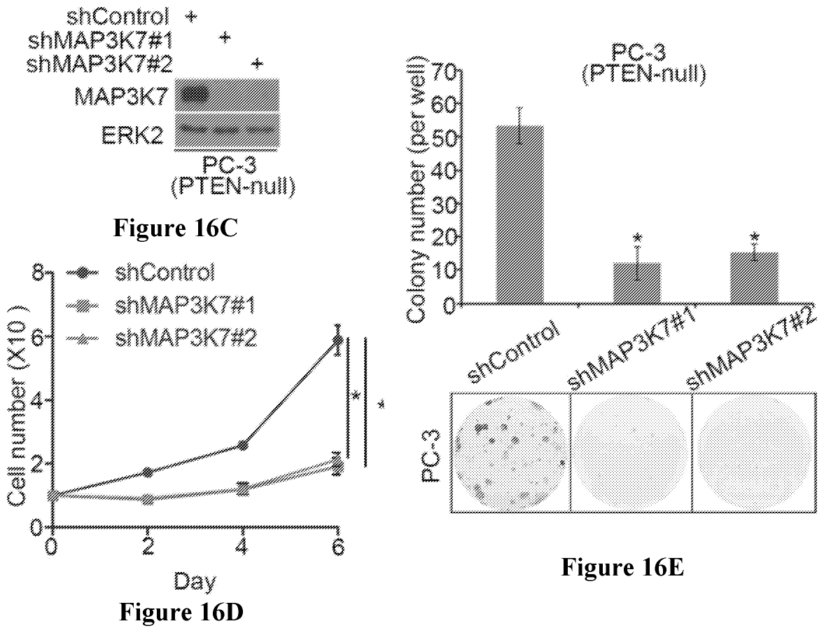

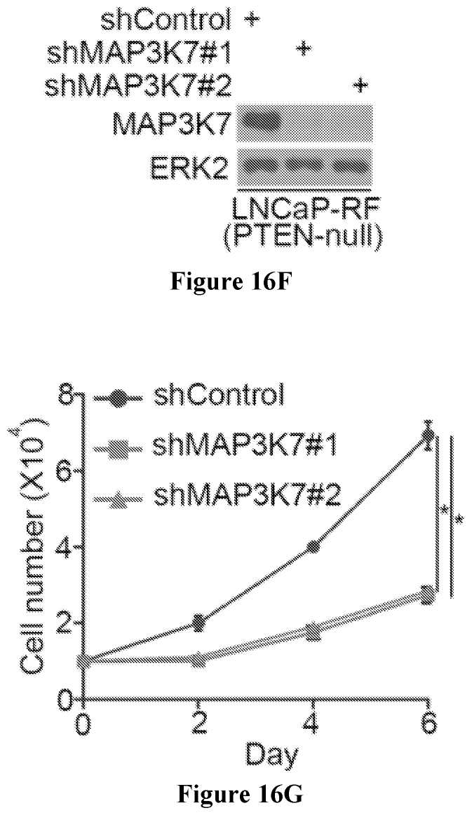

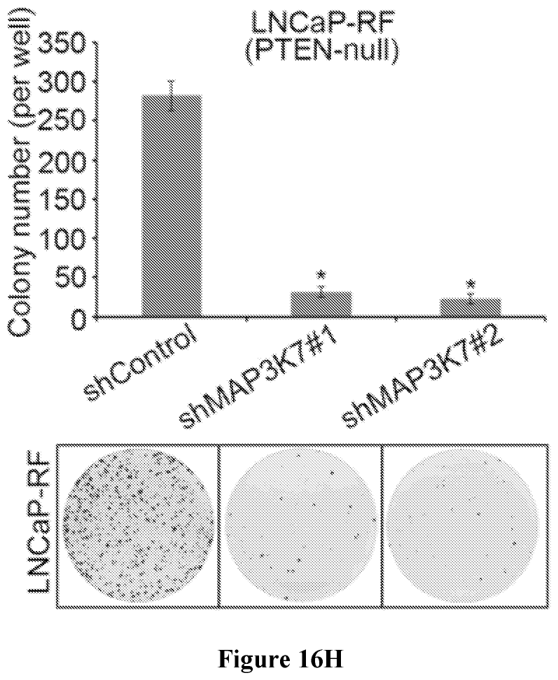

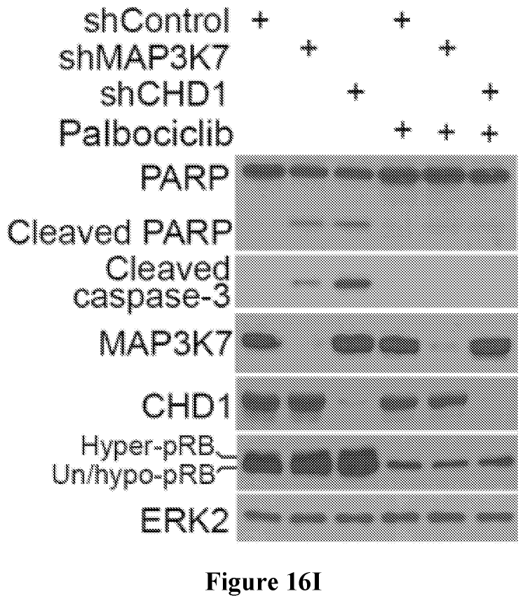

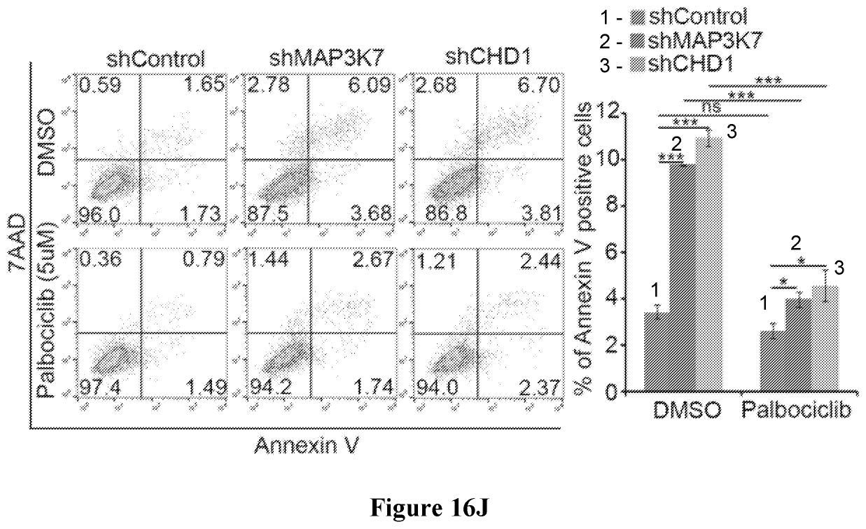

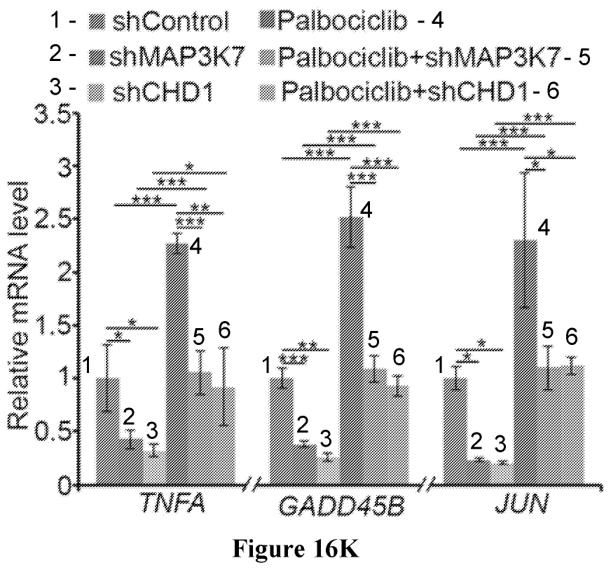

A recent study identifies the chromatin remodeling factor CHD1 as a positive regulator of NFκB and shows CHD1 and PTEN tumor suppressor gene are deleted mutually exclusively in human prostate cancers and their co-deletion is synthetic lethal (Zhao et al., Nature, 542:484-488 ((2017)). The MAP3K7 gene (encoding a kinase also known as TAK1, an upstream activator of NFκB) and PTEN were almost mutually exclusively deleted in multiple cancer types examined (FIGS. 16A and 16B), and their co-depletion also was synthetic lethal (FIGS. 16C-H). Therefore, PTEN/MAP3K7- and PTEN/CHD1-dual-deficient cell lines represent two ideal cell systems to identify bona fide pathways that can rescue synthetic lethality. MAP3K7 or CHD1 was knocked down (KD) in PTEN-null PC-3 cells followed by treatment with an array of small molecule inhibitors for a number of cancer relevant pathways. Drug sensitivity (IC₅₀) analysis revealed that the NFκB inhibitor JSH-23 further increased viability loss in both MAP3K7- and CHD1-KD PC-3 cells (FIG. 14A). Different from other compounds that either exerted opposite responses in MAP3K7- and CHD1-KD cells or had limited protection of cells from being killed, the CDK4/6 inhibitor palbociclib induced a common, large-degree (log 2 scale) increase in the viability in both MAP3K7- and CHD1-KD cells (FIG. 14A). The protective effect was further evident in PARP and caspase-3 protein cleavage and Annexin-V apoptosis assays (FIGS. 161 and 16J). Expression of NFκB target genes including TNFA, GADD45B, and JUN was downregulated by MAP3K7 or CHD1 KD alone, but restored to the steady state level by palbociclib in both cell types (FIG. 16K).

### RB is a Major Downstream Effector of CDK4/6 Signaling

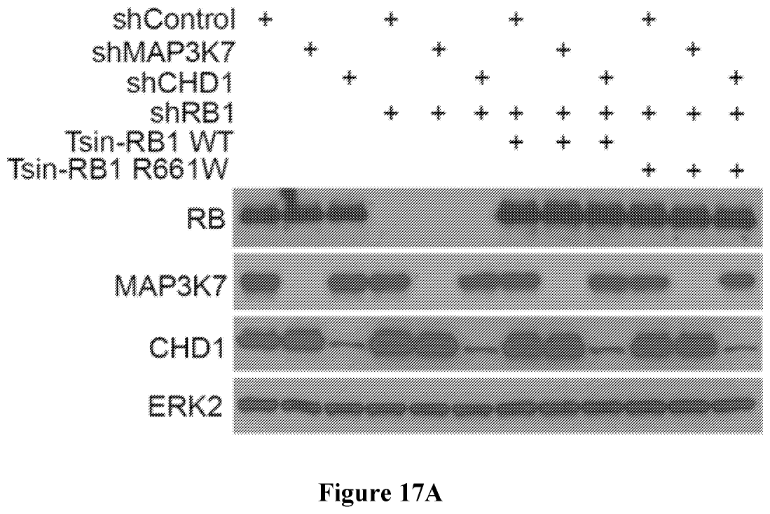

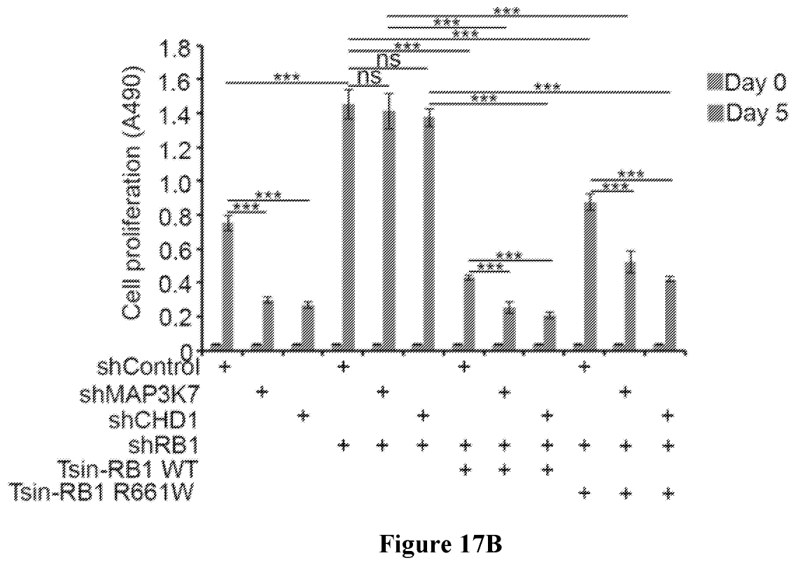

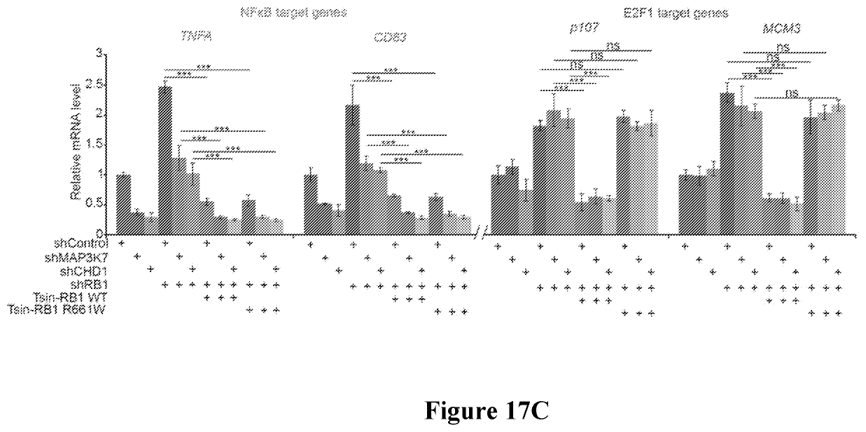

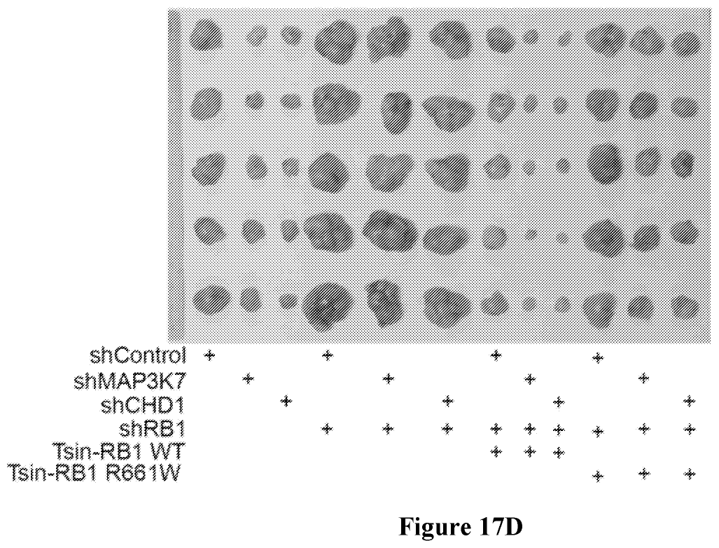

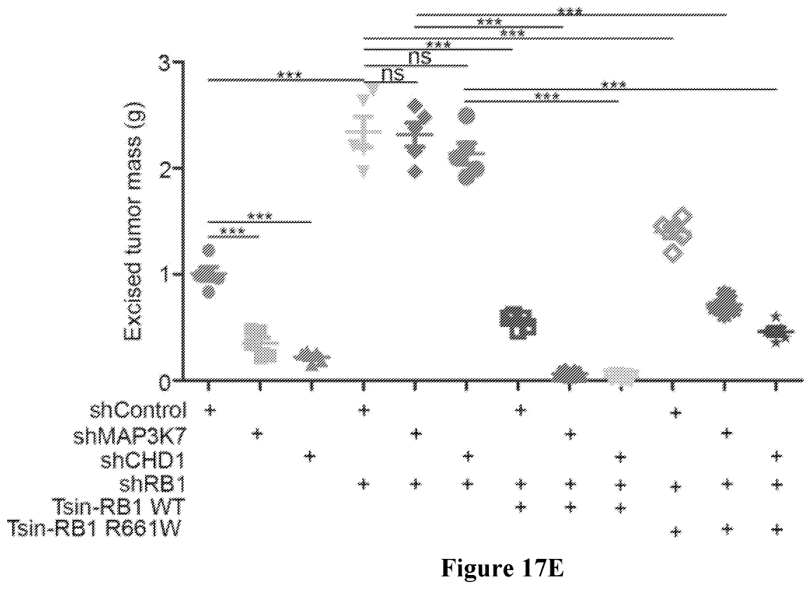

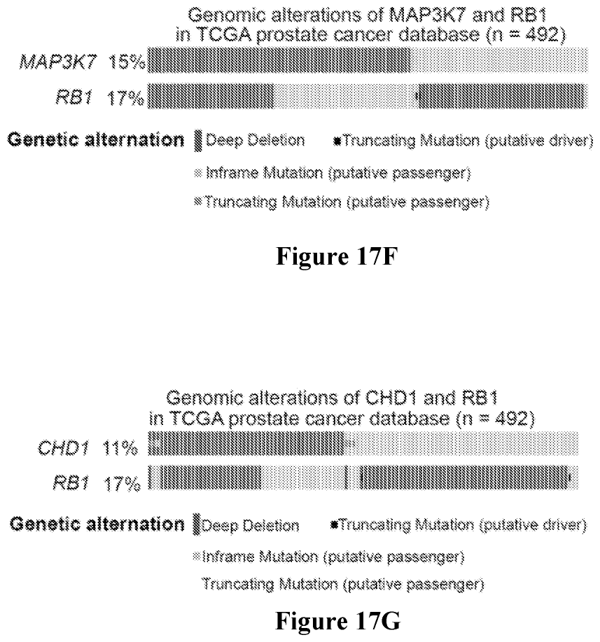

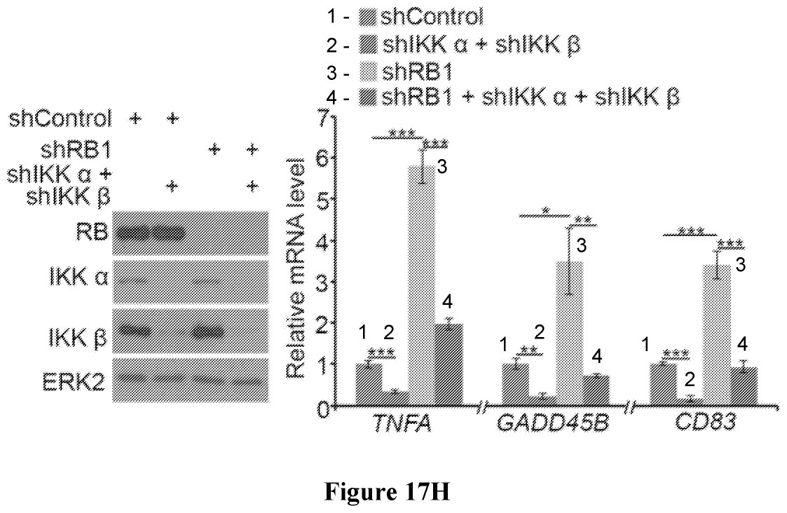

The following was performed to determine whether RB affects the synthetic lethality caused by PTEN/MAP3K7 or PTEN/CHD1 co-deficiency. RB KD blocked MAP3K7 or CHD1 KD-induced PARP and caspase-3 cleavage, apoptotic cell death, and growth inhibition in PTEN-null PC-3 cells (FIGS. 14B, 14C, 17A and 17B), and the same was true for NFκB target gene expression (FIG. 14D). MAP3K7 or CHD1 KD also largely inhibited PTEN-null PC-3 xenograft tumor growth in mice, but such effects were completely abolished by RB KD (FIGS. 14E and 14F).

Rescue experiments revealed that similar to RB-WT, restored expression of R661W (R654W in mouse Rb, an E2F1-binding deficient mutant (Sun et al., Proc. Natl. Acad. Sci. USA, 108:704-709 (2011)) largely blocked MAP3K7 or CHD1 KD-induced inhibition of cell growth and NFκB target gene expression although as expected, the growth inhibitory effect of R661W was not as robust as RB-WT in cell culture and in mice (FIGS. 17A-E), suggesting RB loss-induced protective growth of MAP3K7- or CHD1-deficient tumors is less likely mediated through E2F. These findings were highly disease-relevant since MAP3K7 or CHD1 deletion largely overlapped with RB1 gene deletion in TCGA cohort (FIGS. 17F and 17G). Genetic depletion of IKKα and IKKβ, two NFκB activators downstream of MAP3K7/TAK1 or treatment with IKK inhibitors IKK-16 and ACHP decreased NFκB target gene expression, which was reversed back to the steady state level by RB KD (FIGS. 17H and 17I). These results demonstrated that RB is a major negative regulator of NFκB (FIG. 14G).

RB Interacts with p65 and the Interaction is Enhanced by RB Phosphorylation

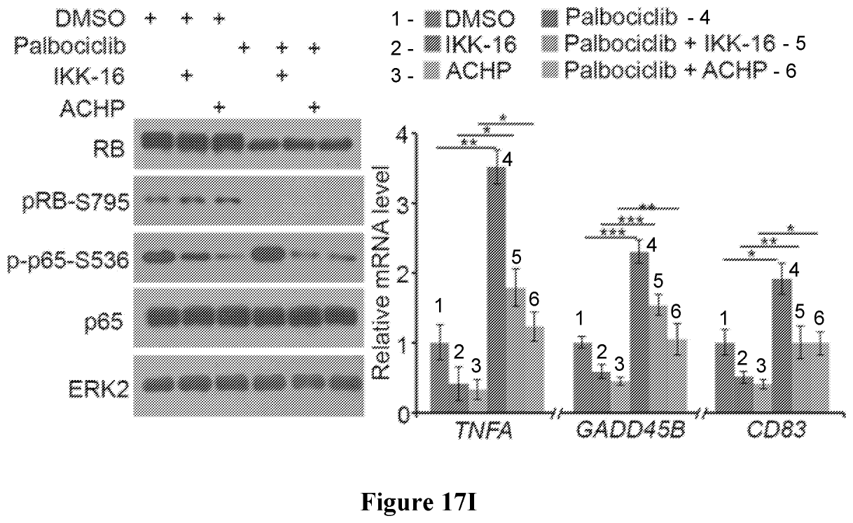

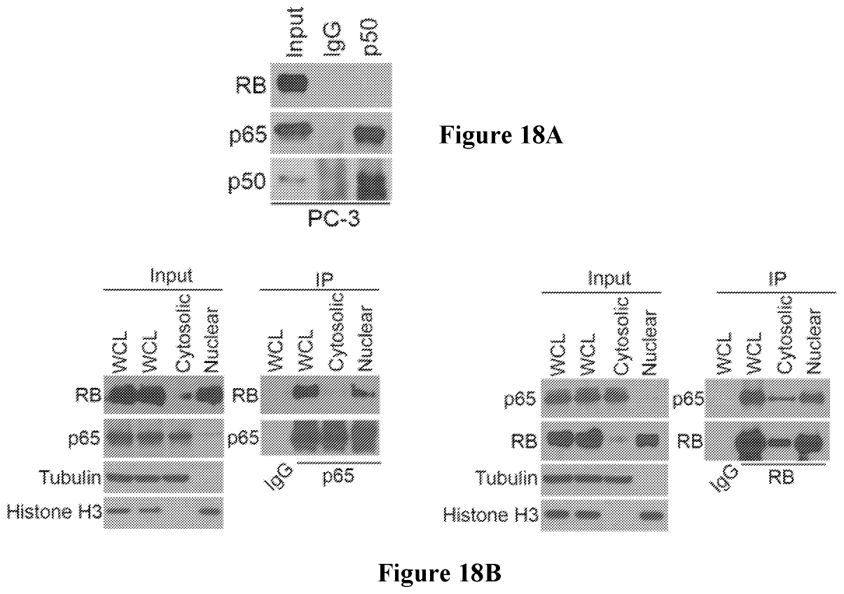

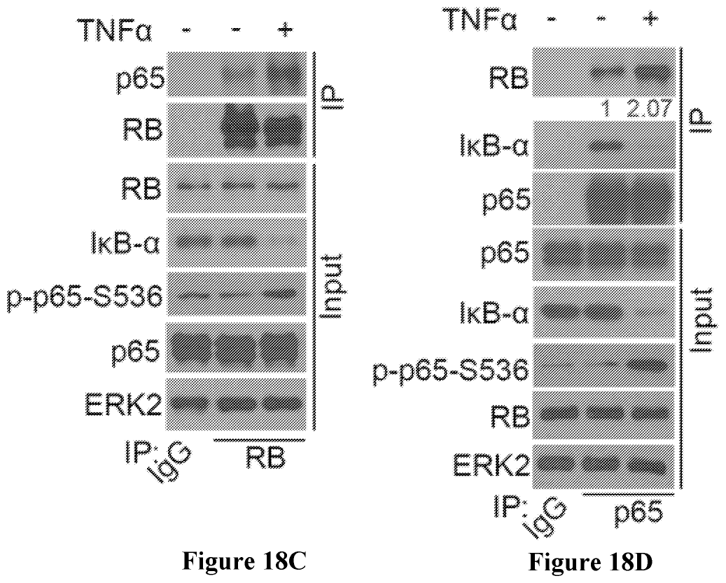

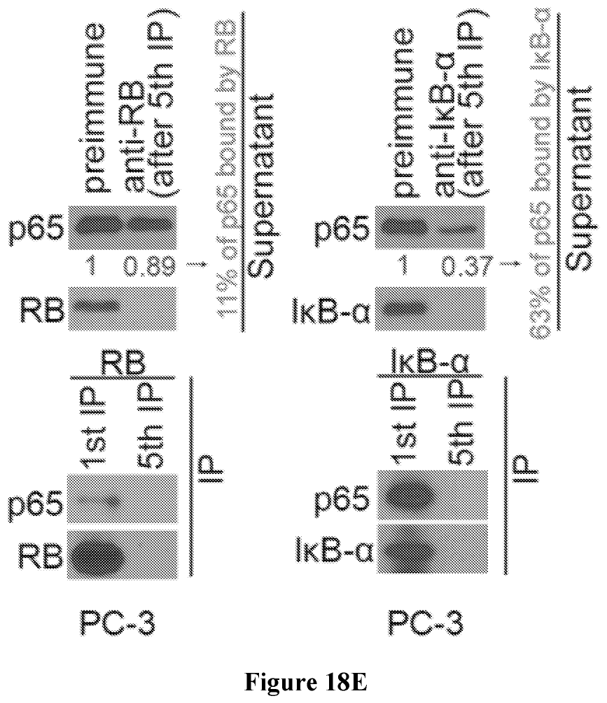

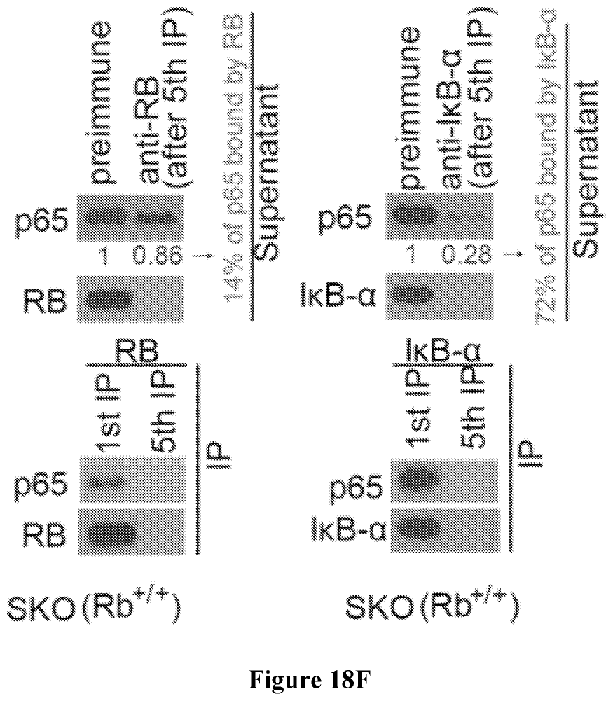

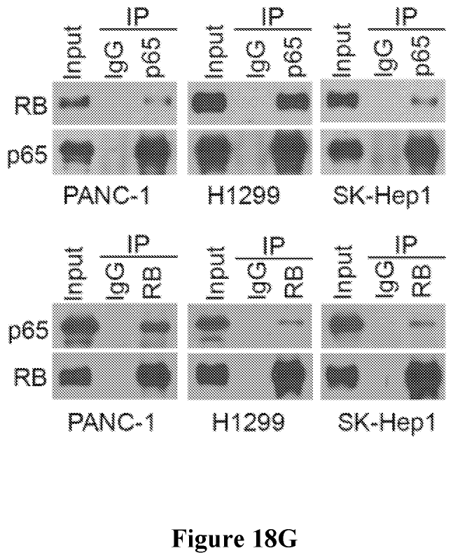

To elucidate the molecular mechanisms by which RB regulates NFκB function, whether RB interacts with NFκB was examined. Co-IP demonstrated that endogenous RB interacted with endogenous p65, but not other NFκB/Rel family proteins RelB, c-Rel, p52, and p50 (FIGS. 2A and 18A). Reciprocal co-IP showed that p65 only interacted with RB, but not other pocket proteins p107 and p130 (FIG. 2B), revealing a specific p65-RB interaction. p65-RB binding primarily occurred in the nucleus (FIG. 18B). In line with the report that TNFα increases nuclear accumulation of p65 (Ariga et al., J. Biol. Chem., 277:24625-24630 (2002)), it increased p65-RB interaction in the nucleus while decreased p65-IκBα interaction (FIGS. 18C and 18D). Immuno-depletion assays revealed that p65 bound to approximately 11% and 14% of RB and 63% and 72% of IkBα in PC-3 and primary Rb-positive Pten single knockout (SKO Rb^{+/+}) cells (Ku et al., Science, 355:78-83 (2017)), respectively (FIGS. 18E and 18F). RB-p65 interaction was also observed in many other cell lines of different cancer types (FIG. 18G).

GST pull down assay revealed that RHD, the DNA binding domain in the N-terminal, but not the C-terminal of p65 specifically bound to RB protein (FIGS. 2C and 2D). RB is a highly phosphorylated protein. Phosphatase treatment decreased RB interaction with GST-p65-N or endogenous p65 (FIGS. 2E and 2F). Nonphosphorylatable RB mutant RBΔCDK, in which fifteen major CDK phosphorylation sites were mutated to alanine (Narasimha et al., Elife 3 (2014)), only had a minimal interaction with p65 compared to RB-WT, while its interaction with E2F1 was increased as expected (FIG. 2G). Thus, RB phosphorylation largely enhanced RB-p65 interaction.

While palbociclib treatment substantially reduced the level of hyperphosphorylated RB, it largely decreased RB-p65 interaction but substantially increased RB-E2F1 interaction (FIG. 2H). Similarly, CDK4 and CDK6 co-knockdown decreased RB-p65 interaction, but increased RB-E2F1 interaction as expected (FIG. 2I). However, RB-p65 interaction was not affected by ectopic expression of E2F binding deficient mutant RB-R661W (FIGS. 9 and 10), suggesting RB-p65 interaction is independent of RB-E2F complex. p65 binds to hyperphosphorylated RB (FIGS. 2F-2I), whereas E2F1 is primarily bound by un- or -hypo-phosphorylated RB.

Although RB-p65 interaction was largely enhanced by RB phosphorylation, certain basal level interaction between nonphosphorylatable RB and p65 was detectable (FIG. 2G). In support of this result, GST pull down assay showed that GST-RB-N, but not GST-RB-M and GST-RB-C retains the ability to bind to p65, although the detected interaction was much weaker compared to 1% input (FIGS. 2J and 2K). Nevertheless, these data indicated RB interacts with p65 via its N-terminal portion. It is worth noting that the amino acid sequences in the N-terminal of RB, p107, and p130 were not conserved, and this region was outside of the E2F-bound pocket domain (FIGS. 9 and 10). These results were consistent with the observations that p65 did not interact with p107, and p130 and p65-RB interaction was independent of RB-E2F1 interaction (FIGS. 2B, 9, and 10).

RB S249/T252 Phosphorylation and ¹⁶¹FQVTV¹⁶⁵ Motif (SEQ ID NO: 258) in p65 are Important for their Interaction

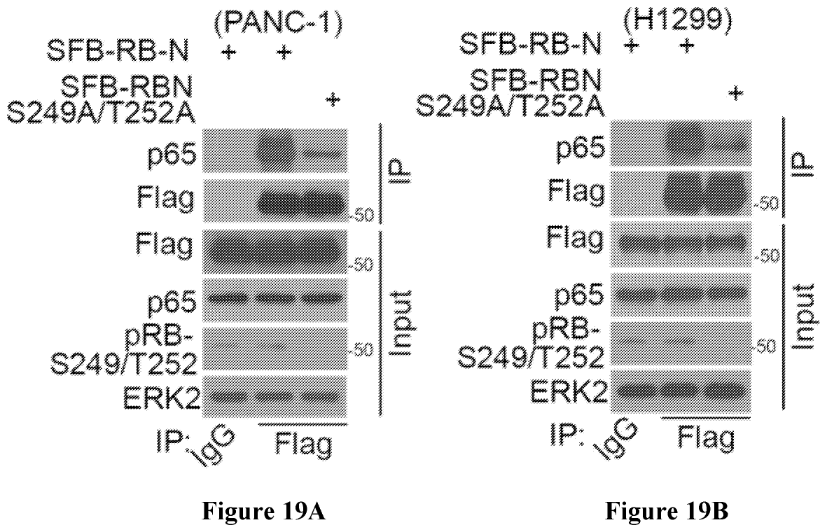

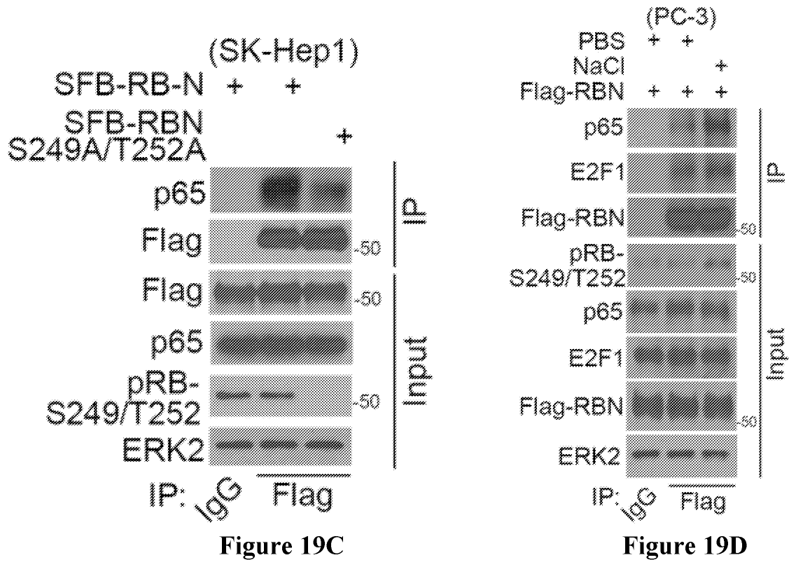

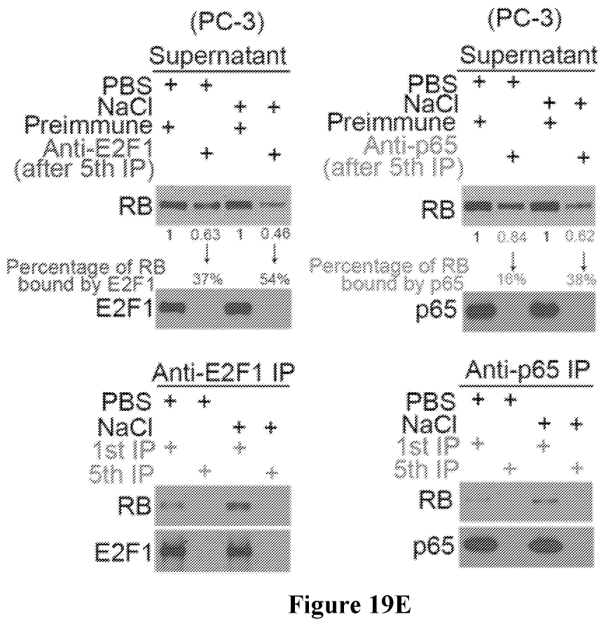

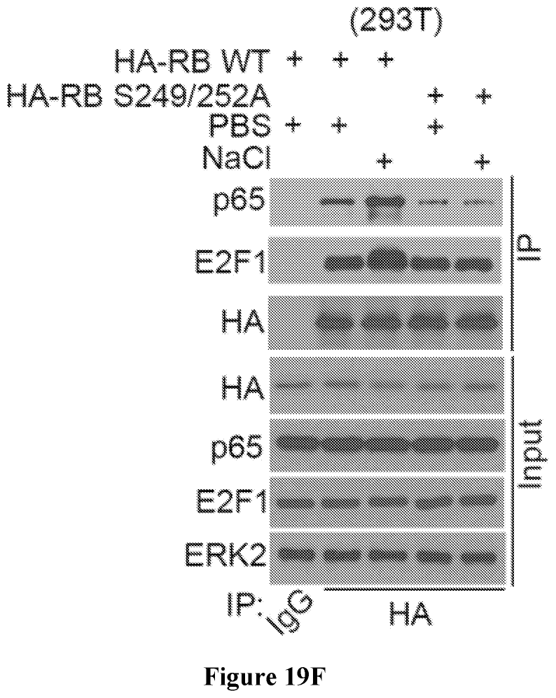

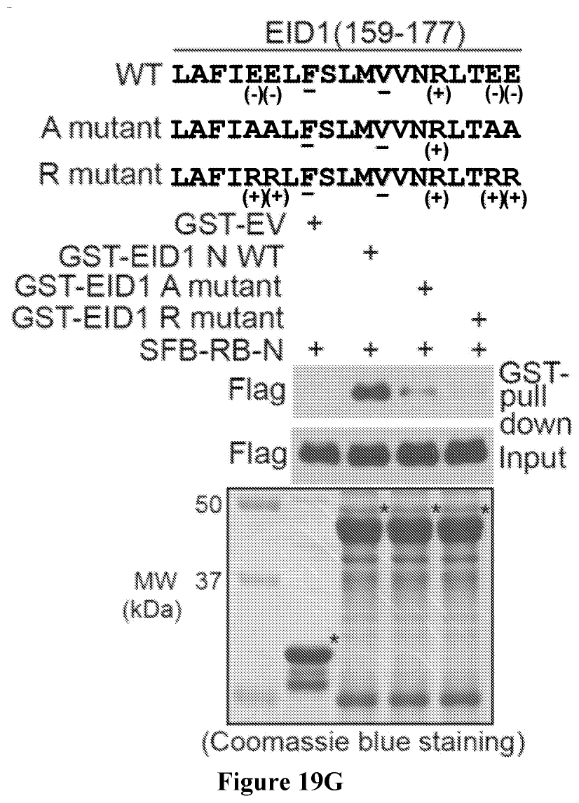

RB-p65 interaction was dependent on RB phosphorylation, and the interaction was largely diminished by CDK4/6 inhibitors or knockdown (FIGS. 2G-2I). The following was performed to determine whether CDK4/6 phosphorylation of RB is involved in RB-p65 interaction. There were four reported CDK4/6 phosphorylation sites in RB-N, two (serine-249 and threonine-252 (S249/T252)) in the arginine-rich linker (R linker) region and the rest (threonine-356 (T356) and threonine-373 (T373)) in the C-terminus (Zarkowska and Mittnacht, J. Biol. Chem., 272:12738-12746 (1997)) (FIG. 3A). These phosphorylation sites were mutated individually into aspartic acid (D), an acidic amino acid closely mimicking phosphorylation. GST pull down assay showed that S249D/T252D, but not other two mutations increased RB-N binding with p65 (FIG. 3A). In agreement with this observation, in vitro protein binding assay using in vitro transcribed and translated p65 protein showed that the N-terminal portion of RB-N(RB-NΔ1 containing S249/T252), but not the C-terminal part (RB-NΔ2 containing T356 and T373) specifically bound to p65 (FIGS. 9 and 10). Moreover, an in vitro kinase assay was performed by inoculating unmutated GST-RB-N or S249/T252 phosphorylation-resistant mutant (S249A/T252A) with the reconstituted CYCLIN D3/CDK4 complex followed by in vitro protein binding assay. S249/T252 phosphorylation on RB-N by CYCLIN D3/CDK4 substantially increased p65-RB-N interaction, but no such effect was observed for S249A/T252A mutant (FIG. 3B). Similar results were obtained with the deletion mutant RB-NΔ²⁴⁹SPRT²⁵², in which S249/T252 residues were deleted (FIG. 10). A RB-N mammalian expression vector containing S, Flag and Biotin-binding-protein (streptavidin)-binding-peptide (SFB) tags was generated. RB-N strongly bound to p65 in various cell lines, but the binding was substantially diminished by S249A/T252A mutation (FIGS. 10, 19A, 19B, and 19C). These data indicated that CDK4/6 phosphorylation of S249/T252 in the R linker region largely enhanced RB-N interaction with p65. It is worth noting that the R linker and S249/T252 phosphorylation sites were not conserved in RB homologs p107 and p130 (FIGS. 9 and 10), consistent with the finding that p65 does not bind to p107 and p130 (FIG. 2B).

The amino acids surrounding S249/T252 in the R linker region are evolutionally conserved from human to mouse (FIG. 10), further highlighting the functional importance of the p65-interaction region in RB-N. To test this notion, an SFB-tagged expression vector was constructed for a S249/T252-containing peptide constituted of twenty-one amino acids in the R linker region (termed as R-linker (RL) peptide) (FIG. 10). p65 was effectively immunoprecipitated by SFB-tagged RL-S249D/T252D, but not SFB alone, and such effect was largely diminished by nonphosphorylatable mutant RL-S249A/T252A (FIG. 10). In vitro protein binding assays demonstrated that the binding between in vitro transcribed and translated RL-S249D/T252D peptide, and GST-p65-N was much stronger compared to phosphorylation-resistant mutant RL-S249/T252A (FIG. 10). Similar to a previous report that osmotic shock induces p38-dependent S249/T252 phosphorylation of RB and enhances RB-N interaction with E2F1 (Gubern et al., Mol. Cell, 64:25-36 (2016)), sodium chloride treatment also increased p65-RB interaction in a S249/T252-phosphorylation-dependent manner (FIGS. 19D-F). A small RB-N-derived phospho-mimetic peptide that can strongly bind to p65 was identified.

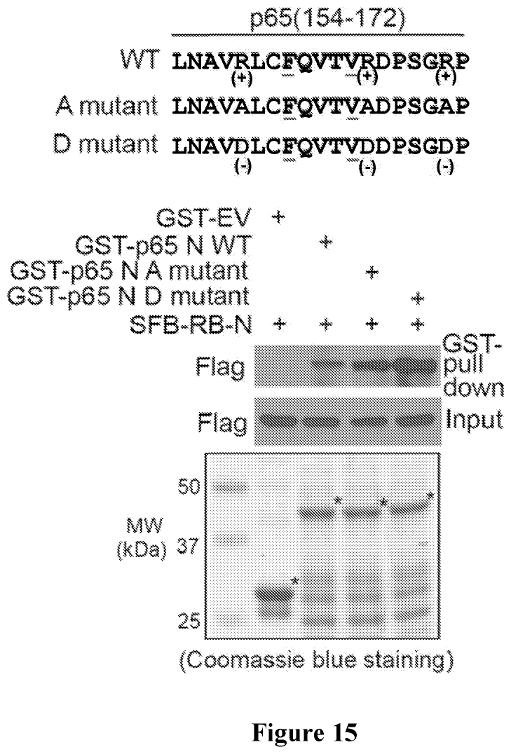

A previous study identified an FxxxV (¹⁶⁶FSLMV¹⁷⁰ (SEQ ID NO: 286)) motif-centered region in a protein termed E1A-like inhibitor of differentiation-1 (EID1) that is important for its interaction with RB-N(Hassler et al., Mol. Cell, 28:371-385 (2007)). p65 was found to also harbor an evolutionally conserved FxxxV (¹⁶¹FQVTV¹⁶⁵ (SEQ ID NO: 258)) motif within the RB-N-binding region (FIG. 3C). Like EID1, deletion of ¹⁶¹FQVTV¹⁶⁵ (SEQ ID NO: 258) in p65-N (p65-NΔFV) largely diminished RB-p65 interaction (FIG. 3D). However, different from the previous report that the EID1-RB-N interaction was abrogated by CDK-mediated S249/T252 phosphorylation (Hassler et al., Mol. Cell, 28:371-385 (2007)), p65-RB-N interaction was largely enhanced by S249/T252 phosphorylation (FIG. 3B). After carefully examining the amino acid sequences in the interaction motifs in these three proteins, it was noticed that the R linker in RB-N is an arginine (positive charge)-rich region, and that there are several positively-charged arginine residues surrounding the ¹⁶¹FQVTV¹⁶⁵ motif (SEQ ID NO: 258) in p65 whereas more negatively-charged (e.g. glutamic acid) than positively-charged amino acids were present adjacent to the ¹⁶⁶FSLMV¹⁷⁰ motif (SEQ ID NO: 286) in EID1 (FIGS. 3E and 3F). Based upon these observations, a model was envisioned wherein introduction of negative charge by S249/T252 phosphorylation may allow otherwise fully positively-charged RB-N to bind to positively-charged ¹⁶¹FQVTV¹⁶⁵ (SEQ ID NO: 258)-containing region in p65. In contrast, the same phosphorylation in the R linker impedes RB-N interaction with the negatively-charged ¹⁶⁶FSLMV¹⁷⁰ (SEQ ID NO: 286)-centered region in EID1 (FIGS. 3E-3G). To experimentally test this hypothesis, positively-charged arginine residues surrounding the ¹⁶¹FQVTV¹⁶⁵ motif (SEQ ID NO: 258) in p65-N were converted to alanine (A mutant) or negatively-charged aspartic acid (D mutant), but mutated negatively-charged glutamic acid residues next to the ¹⁶⁶FSLMV¹⁷⁰ motif (SEQ ID NO: 286) in EID1-N were converted to alanine (A mutant) or positively-charged arginine (R mutant) (FIGS. 15 and 19G, top panels). GST recombinant proteins of these mutants and the WT control were purified for pull-down assay. The interaction between p65-N D mutant and RB-N was much stronger than that of WT or A mutant (FIG. 15, lower panel). In striking contrast, the interaction of EID1-N D mutant with RB-N was much weaker than that of WT or A mutant (FIG. 19G, lower panel). These data indicate that charge composition of amino acids in RB interacting region in client proteins such as p65 and EID1 is the determinants for RB binding.

### RB Regulates Expression of a Subset of NFκB Target Genes

Specific interaction of RB with p65, but not other NFκB proteins suggests that RB may partially regulate NFκB transcription program. RNA-seq analysis was performed in RB-knockdown and palbociclib-treated PC-3 cells. A subset of genes whose expression was commonly down- or up-regulated by RB knockdown and palbociclib in three replicates was identified (one inconsistent replicate in shControl cells was excluded for analysis) (FIGS. 4A and 11). Ingenuity pathway analysis (IPA) of 897 commonly upregulated genes showed that among the signaling pathways significantly affected by both RB knockdown and palbociclib were T-cell regulatory pathway genes including CD274 (gene symbol for PD-L1 or B7-H1), expression of which is known to be regulated by NFκB (Bouillez et al., Oncogene, 36:4037-4046 (2017); and Peng et al., Cancer Res., 75:5034-5045 (2015)) (FIGS. 4A-4C). Gene set enrichment analysis (GSEA) revealed that the upregulated genes also significantly overlapped with TNFα-NFκB pathway, epithelial-to-mesenchymal transition (EMT), hypoxia, and ultraviolet (UV) response genes (FIGS. 4C and 4D). RNA-seq results for the genes commonly upregulated by RB knockdown and palbociclib were exemplified by the NFκB targets CD274 (PD-L1), GADD45B, NR4A2, and CD83 (FIG. 4E). Upregulation of these genes was further verified by reverse transcription-quantitative polymerase chain reaction (RT-qPCR) (FIG. 4F). These data indicated that expression of a subset of NFκB target genes including PD-L1 was selectively regulated by RB.

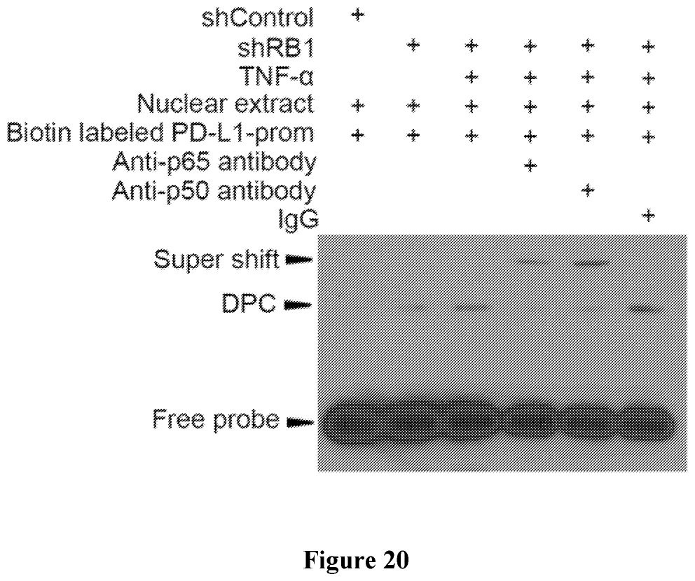

Chromatin immunoprecipitation-coupled quantitative PCR (ChIP-qPCR) analysis revealed that p65 protein readily bound to the genomic loci of the RB-affected NFκB target genes including PD-L1, and p65 occupancy at these loci was substantially increased by RB knockdown or palbociclib in PC-3 cells (FIG. 4G). Transcription factor DNA-binding sequence analysis revealed that there is a putative NFκB binding sequence (NBS) in the promoter of PD-L1 and other NFκB target genes examined (FIG. 11). Electrophoretic mobility shift assay (EMSA) was performed using biotin-labeled NBS from the PD-L1 promoter (PD-L1-Prom) as probe (FIGS. 11 and 20). A DNA-protein complex (DPC) was detected in nuclear extract of PC-3 cells treated with vehicle or control shRNA (FIGS. 11 and 20). The DPC level was largely augmented by RB knockdown or palbociclib treatment alone and further enhanced by TNFα (FIGS. 11 and 20). A super-shifted DPC was detected in reactions inoculated with anti-p65 or anti-p50 antibodies, but not non-specific IgG (FIG. 20). A functional NFκB binding site was identified in the PD-L1 promoter at which p65 binding is regulated by the CDK4/6-RB signaling axis.

### Involvement of RB S249/T252 Phosphorylation in Regulation of PD-L1 Expression

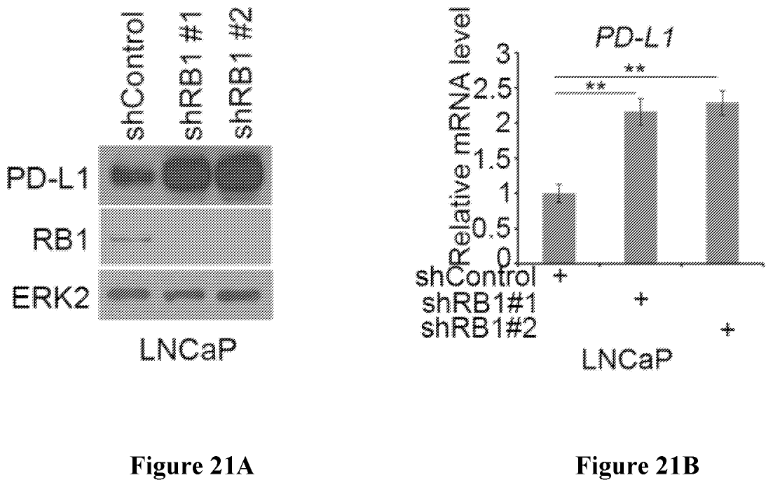

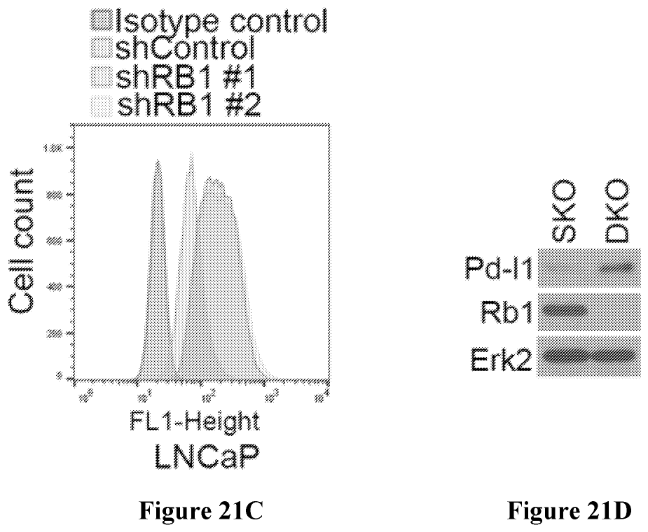

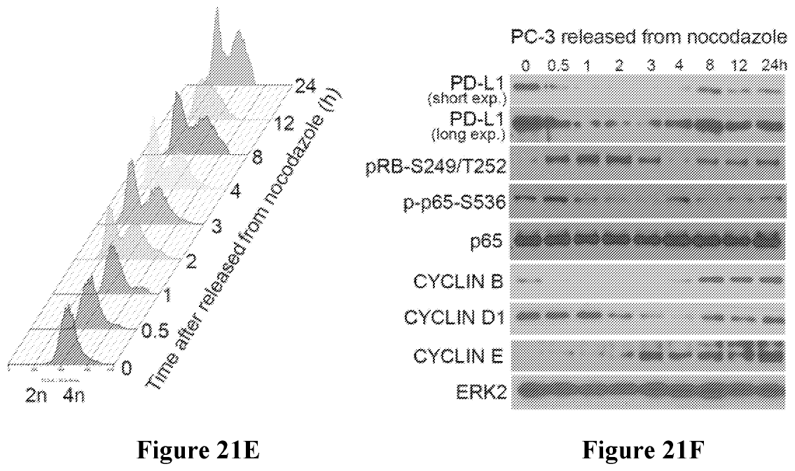

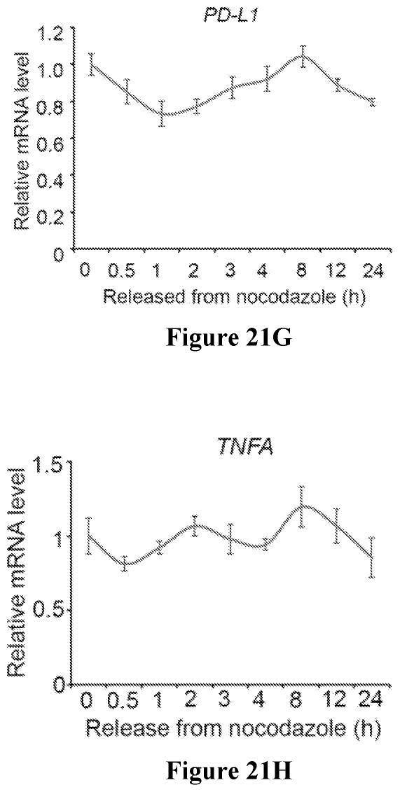

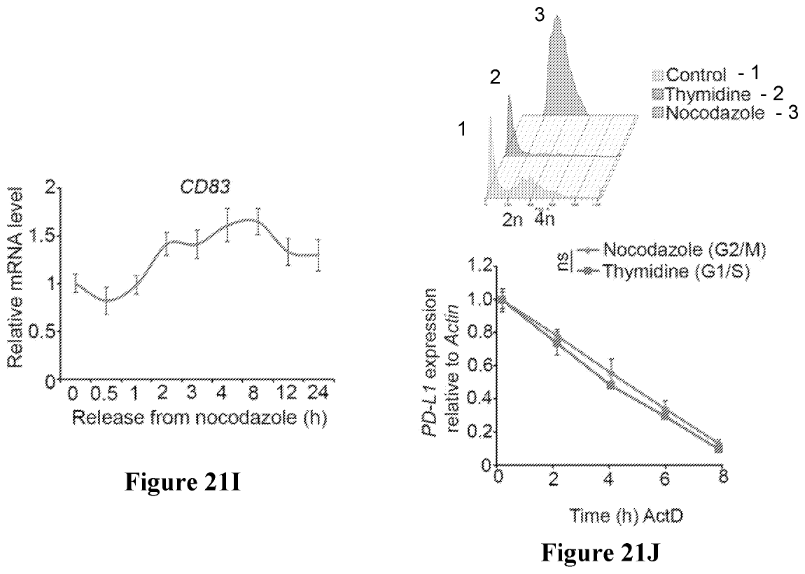

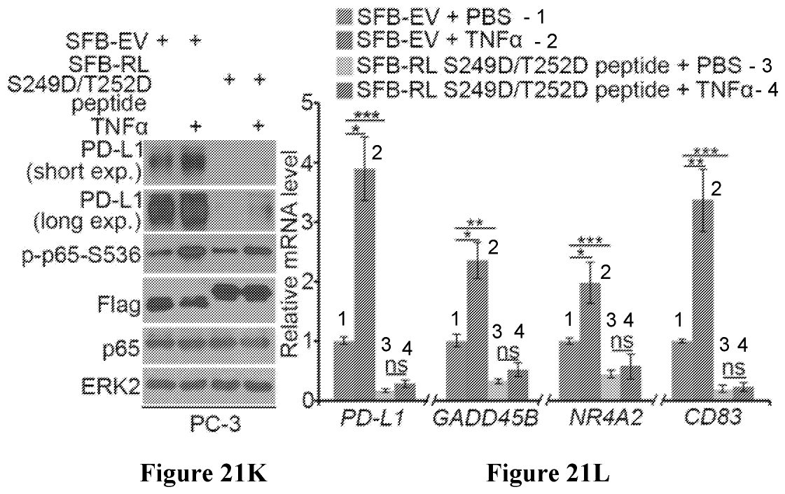

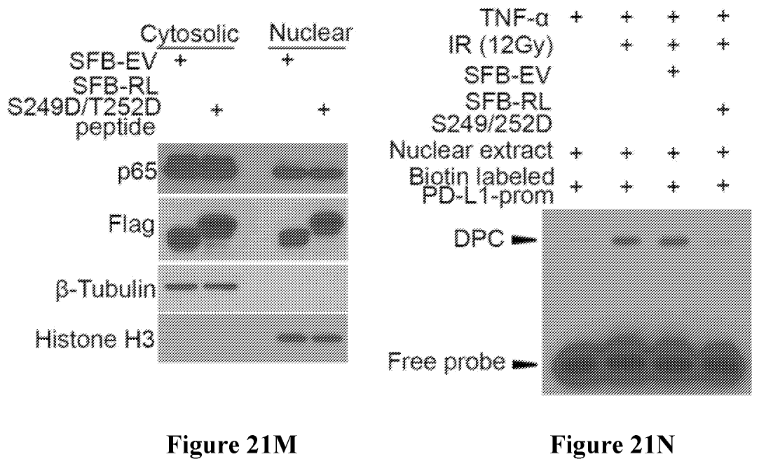

T-cell responses can be reactivated by blockade of PD-1/PD-L1 interaction with agents such as PD-1 and PD-L1 antibodies and utilized for cancer treatment (Iwai et al., Proc. Natl. Acad. Sci. USA, 99:12293-12297 (2002); Topalian et al., Curr. Opin. Immunol., 24:207-212 (2012); Zhu and Chen, Curr. Opin. Investig. Drugs., 4:691-695 (2003)). The following was performed to determine whether RB regulation of PD-L1 expression is affected by RB phosphorylation and whether such regulatory mechanism can be harnessed for cancer therapy. Consistent with the finding that RB depletion upregulated PD-L1 mRNA expression (FIGS. 4E and 4F), RB knockdown by two independent shRNAs in PC-3 cells invariably increased PD-L1 protein expression as demonstrated by western blot, FACS, and immunofluorescent cytochemistry (FIGS. 1 and 5), and similar results were obtained in another prostate cancer cell line LNCaP (FIGS. 21A-21C). Pd-l1 expression was further examined in murine prostate cancer cell lines established from prostate-specific Rb1 and Pten double knockout (DKO) and Pten single knockout (SKO) mice (Ku et al., Science, 355:78-83 (2017)). Pd-l1 expression at both mRNA and protein level was much higher in Rb-deficient DKO cells than that in Rb-proficient SKO cells (FIGS. 21D and 1). The findings from cell lines are fully supported by the data in a cohort of metastatic castration-resistant prostate cancer (mCRPC) patients that PD-L1 mRNA levels were significantly higher in RB1 homozygously-deleted prostate tumors compared with tumors without RB1 deletion (FIG. 1). Notably, RB knockdown-induced upregulation of PD-L1 was abrogated by p65 knockdown in PC-3 cells (FIGS. 1 and 5). These results demonstrated that RB represses PD-L1 expression in an NFκB-dependent manner.

The following was performed to examine whether PD-L1 expression is regulated by RB phosphorylation. Ectopic expression of RB WT substantially decreased PD-L1 expression, and this effect was largely attenuated in cells expressing the phosphorylation-resistant mutant RBΔCDK (FIGS. 1 and 5). Notably, PD-L1 expression level was lower in RBΔCDK-expressing cells than control cells (FIGS. 1 and 5), which was consistent with the presence of the certain basal level interaction between RBΔCDK and p65 (FIG. 2G). Expression of RB-N, the p65 interacting region, was also sufficient to inhibit PD-L1 expression at both mRNA and protein levels, but this effect was largely diminished by S249A/T252A mutation (FIGS. 1 and 5), highlighting the importance of CDK4/6 phosphorylation of RB in repression of PD-L1 expression. PD-L1 protein expression was much lower in cells at G1/S phase compared to that in cells at G2/M phase (FIG. 21). Similar to the previous finding in B cells (Inoue et al., NPJ Syst. Biol. Appl., 2:16024 (2016)), expression of NFκB target genes including PD-L1 was cell cycle regulated and occurred at mRNA level (FIGS. S6I-K), but the alteration was not mediated by RNA stability, at least in G1/S and G2/M phases examined (FIG. 21). In support of the protein binding results (FIG. 10), expression of the small phospho-mimetic peptide derived from the p65-binding motif in RB-N(RL-S249/T252D peptide) largely inhibited PD-L1 expression in p65-proficient cells, but little or no effect was observed in p65-depleted cells (FIGS. 1 and 5). Expression of this phospho-mimetic peptide also blocked TNFα-induced expression of NFκB target genes (FIG. 21), and such effect was not due to its sequestration of p65 proteins in the cytoplasm, but rather mediated by its inhibition of p65 binding to the cognate DNA in the PD-L1 promoter (FIG. 21). This peptide also inhibited PD-L1 expression in several cell lines of different cancer types (FIGS. 12 and 13), indicating that this peptide was bioactive in a broad spectrum of cancer types. A small phospho-mimetic peptide of RB-N was identify that can inhibit PD-L1 expression in a p65-dependent manner.

RB phosphorylation at S249/T252, but not total RB protein was generally corrected with PD-L1 protein expression in an array of prostatic cell lines (FIGS. 12 and 13). S249/T252-phosphorylated RB (pRB-S249/T252) and PD-L1 expression was further examined in patient samples by performing immunohistochemistry (IHC) on a tissue microarray (TMA) containing a cohort of metastatic castration-resistant prostate cancer (mCRPC) specimens (n=145 TMA elements). IHC staining was evaluated by measuring both staining intensity and percentage of positive cells (Table 6). Representative IHC images displaying high and low/lost staining of PD-L1 and pRB-S249/T252 are shown in FIGS. 1 and 5. In this cohort, 65 out of 145 (44.8%) TMA specimens were PD-L1 positive, but only 17 out of 145 (11.7%) TMA specimens expressed moderate to high levels of PD-L1 protein on cell surface. Detection of low rate expression of PD-L1 in this cohort is consistent with previous reports (Gevensleben et al., Clin. Cancer Res., 22:1969-1977 (2016); and Massari et al., Target Oncol., 11:345-351 (2016)). Further analysis showed that PD-L1 protein expression inversely correlated with pRB-S249/T252 level among these patients (Pearson's product-moment correlation r=−0.389, P=1.33e−06) (FIG. 5M). These data suggested regulation of PD-L1 expression by S249/T252-phosphorylated RB may also occur in mCRPC patients.

TABLE 6
Raw staining index scores for PD-L1 and pRB-S249/T252 IHC of mCRPC
human tissue microarray.

| | PD-L1 | | | pRB-S249/T252 | | |
|---|---|---|---|---|---|---|
| | | | Staining | | | Staining |
| | Intensity | Percentage | Index | Intensity | Percentage | Index |
| 1 | 1 | 0.05 | 0.05 | 2 | 0.6 | 1.2 |
| 2 | 2 | 0.15 | 0.3 | 3 | 0.4 | 1.2 |
| 3 | 0 | 0 | 0 | 1 | 0.5 | 0.5 |
| 4 | 0 | 0 | 0 | 2 | 0.4 | 0.8 |
| 5 | 0 | 0 | 0 | 1 | 0.6 | 0.6 |
| 6 | 1 | 0.1 | 0.1 | 3 | 0.6 | 1.8 |
| 7 | 0 | 0 | 0 | 2 | 0.7 | 1.4 |
| 8 | 0 | 0 | 0 | 2 | 0.7 | 1.4 |
| 9 | 0 | 0 | 0 | 2 | 0.9 | 1.8 |
| 10 | 0 | 0 | 0 | 3 | 0.2 | 0.6 |
| 11 | 0 | 0 | 0 | 2 | 0.9 | 1.8 |
| 12 | 3 | 0.2 | 0.6 | 2 | 0.7 | 1.4 |
| 13 | 0 | 0 | 0 | 2 | 0.8 | 1.6 |
| 14 | 0 | 0 | 0 | 2 | 0.7 | 1.4 |
| 15 | 0 | 0 | 0 | 2 | 0.05 | 0.1 |
| 16 | 3 | 0.1 | 0.3 | 3 | 0.5 | 1.5 |
| 17 | 3 | 0.7 | 2.1 | 0 | 0 | 0 |
| 18 | 3 | 0.7 | 2.1 | 0 | 0 | 0 |
| 19 | 2 | 0.2 | 0.4 | 2 | 0.9 | 1.8 |
| 20 | 0 | 0 | 0 | 2 | 0.6 | 1.2 |
| 21 | 0 | 0 | 0 | 3 | 0.6 | 1.8 |
| 22 | 0 | 0 | 0 | 2 | 0.3 | 0.6 |
| 23 | 2 | 0.4 | 0.8 | 2 | 0.1 | 0.2 |
| 24 | 0 | 0 | 0 | 2 | 0.3 | 0.6 |
| 25 | 0 | 0 | 0 | 2 | 0.1 | 0.2 |
| 26 | 0 | 0 | 0 | 2 | 0.2 | 0.4 |
| 27 | 0 | 0 | 0 | 1 | 0.2 | 0.2 |
| 28 | 3 | 0.1 | 0.3 | 2 | 0.6 | 1.2 |
| 29 | 0 | 0 | 0 | 0 | 0 | 0 |
| 30 | 0 | 0 | 0 | 1 | 0.1 | 0.1 |
| 31 | 0 | 0 | 0 | 0 | 0 | 0 |
| 32 | 2 | 0.4 | 0.8 | 2 | 0.2 | 0.4 |
| 33 | 1 | 0.1 | 0.1 | 2 | 0.5 | 1 |
| 34 | 0 | 0 | 0 | 2 | 0.5 | 1 |
| 35 | 0 | 0 | 0 | 2 | 0.5 | 1 |
| 36 | 0 | 0 | 0 | 3 | 0.4 | 1.2 |
| 37 | 0 | 0 | 0 | 2 | 0.5 | 1 |
| 38 | 0 | 0 | 0 | 2 | 0.3 | 0.6 |
| 39 | 0 | 0 | 0 | 0 | 0 | 0 |
| 40 | 3 | 0.1 | 0.3 | 3 | 0.7 | 2.1 |
| 41 | 2 | 0.05 | 0.1 | 3 | 0.6 | 1.8 |
| 42 | 0 | 0 | 0 | 2 | 0.3 | 0.6 |
| 43 | 0 | 0 | 0 | 2 | 0.9 | 1.8 |
| 44 | 0 | 0 | 0 | 0 | 0 | 0 |
| 45 | 3 | 0.15 | 0.45 | 3 | 0.5 | 1.5 |
| 46 | 0 | 0 | 0 | 3 | 0.4 | 1.2 |
| 47 | 3 | 0.6 | 1.8 | 0 | 0 | 0 |
| 48 | 0 | 0 | 0 | 2 | 0.7 | 1.4 |
| 49 | 3 | 0.3 | 0.9 | 0 | 0 | 0 |
| 50 | 0 | 0 | 0 | 3 | 0.4 | 1.2 |
| 51 | 3 | 0.4 | 1.2 | 1 | 0.1 | 0.1 |
| 52 | 2 | 0.3 | 0.6 | 0 | 0 | 0 |
| 53 | 2 | 0.3 | 0.6 | 1 | 0.6 | 0.6 |
| 54 | 0 | 0 | 0 | 1 | 0.9 | 0.9 |
| 55 | 0 | 0 | 0 | 2 | 0.5 | 1 |
| 56 | 0 | 0 | 0 | 2 | 0.9 | 1.8 |
| 57 | 0 | 0 | 0 | 2 | 0.9 | 1.8 |
| 58 | 1 | 0.1 | 0.1 | 1 | 0.7 | 0.7 |
| 59 | 2 | 0.2 | 0.4 | 1 | 0.6 | 0.6 |
| 60 | 2 | 0.3 | 0.6 | 2 | 0.6 | 1.2 |
| 61 | 2 | 0.3 | 0.6 | 0 | 0 | 0 |
| 62 | 3 | 0.4 | 1.2 | 1 | 0.3 | 0.3 |
| 63 | 3 | 0.2 | 0.6 | 1 | 0.3 | 0.3 |
| 64 | 0 | 0 | 0 | 3 | 0.7 | 2.1 |
| 65 | 1 | 0.1 | 0.1 | 3 | 0.7 | 2.1 |
| 66 | 1 | 0.2 | 0.2 | 3 | 0.7 | 2.1 |
| 67 | 0 | 0 | 0 | 3 | 0.4 | 1.2 |
| 68 | 0 | 0 | 0 | 2 | 0.4 | 0.8 |
| 69 | 1 | 0.9 | 0.9 | 2 | 0.4 | 0.8 |
| 70 | 2 | 0.1 | 0.2 | 2 | 0.6 | 1.2 |
| 71 | 2 | 0.1 | 0.2 | 3 | 0.5 | 1.5 |
| 72 | 0 | 0 | 0 | 3 | 0.5 | 1.5 |
| 73 | 3 | 0.7 | 2.1 | 1 | 0.4 | 0.4 |
| 74 | 1 | 0.2 | 0.2 | 3 | 0.8 | 2.4 |
| 75 | 2 | 0.1 | 0.2 | 3 | 0.9 | 2.7 |
| 76 | 3 | 0.6 | 1.8 | 1 | 0.4 | 0.4 |
| 77 | 2 | 0.5 | 1 | 0 | 0 | 0 |
| 78 | 0 | 0 | 0 | 1 | 0.9 | 0.9 |
| 79 | 1 | 0.2 | 0.2 | 1 | 0.1 | 0.1 |
| 80 | 2 | 0.1 | 0.2 | 2 | 0.9 | 1.8 |
| 81 | 1 | 0.05 | 0.05 | 2 | 0.8 | 1.6 |
| 82 | 0 | 0 | 0 | 2 | 0.9 | 1.8 |
| 83 | 0 | 0 | 0 | 2 | 0.8 | 1.6 |
| 84 | 1 | 0.1 | 0.1 | 1 | 0.6 | 0.6 |
| 85 | 0 | 0 | 0 | 2 | 0.3 | 0.6 |
| 86 | 0 | 0 | 0 | 0 | 0 | 0 |
| 87 | 0 | 0 | 0 | 0 | 0 | 0 |
| 88 | 3 | 0.3 | 0.9 | 0 | 0 | 0 |
| 89 | 0 | 0 | 0 | 3 | 0.7 | 2.1 |
| 90 | 0 | 0 | 0 | 2 | 0.5 | 1 |
| 91 | 1 | 0.05 | 0.05 | 2 | 0.8 | 1.6 |
| 92 | 0 | 0 | 0 | 0 | 0 | 0 |
| 93 | 1 | 0.3 | 0.3 | 3 | 0.7 | 2.1 |
| 94 | 1 | 0.2 | 0.2 | 3 | 0.8 | 2.4 |
| 95 | 1 | 0.2 | 0.2 | 2 | 0.7 | 1.4 |
| 96 | 3 | 0.3 | 0.9 | 1 | 0.3 | 0.3 |
| 97 | 3 | 0.3 | 0.9 | 2 | 0.8 | 1.6 |
| 98 | 1 | 0.1 | 0.1 | 1 | 0.8 | 0.8 |
| 99 | 0 | 0 | 0 | 2 | 0.6 | 1.2 |
| 100 | 0 | 0 | 0 | 3 | 0.4 | 1.2 |
| 101 | 2 | 0.2 | 0.4 | 0 | 0 | 0 |
| 102 | 2 | 0.2 | 0.4 | 0 | 0 | 0 |
| 103 | 0 | 0 | 0 | 3 | 0.4 | 1.2 |
| 104 | 1 | 0.3 | 0.3 | 2 | 0.9 | 1.8 |
| 105 | 3 | 0.1 | 0.3 | 2 | 0.7 | 1.4 |
| 106 | 0 | 0 | 0 | 2 | 0.9 | 1.8 |
| 107 | 0 | 0 | 0 | 3 | 0.4 | 1.2 |
| 108 | 0 | 0 | 0 | 3 | 0.9 | 2.7 |
| 109 | 0 | 0 | 0 | 3 | 0.8 | 2.4 |
| 110 | 0 | 0 | 0 | 3 | 0.9 | 2.7 |
| 111 | 0 | 0 | 0 | 3 | 0.8 | 2.4 |
| 112 | 0 | 0 | 0 | 2 | 0.9 | 1.8 |
| 113 | 0 | 0 | 0 | 2 | 0.6 | 1.2 |
| 114 | 0 | 0 | 0 | 2 | 0.3 | 0.6 |
| 115 | 2 | 0.65 | 1.3 | 0 | 0 | 0 |
| 116 | 2 | 0.55 | 1.1 | 0 | 0 | 0 |
| 117 | 0 | 0 | 0 | 2 | 0.8 | 1.6 |
| 118 | 1 | 0.2 | 0.2 | 3 | 0.8 | 2.4 |
| 119 | 2 | 0.3 | 0.6 | 3 | 0.7 | 2.1 |
| 120 | 2 | 0.3 | 0.6 | 2 | 0.9 | 1.8 |
| 121 | 3 | 0.1 | 0.3 | 3 | 0.2 | 0.6 |
| 122 | 0 | 0 | 0 | 1 | 0.2 | 0.2 |
| 123 | 2 | 0.6 | 1.2 | 1 | 0.05 | 0.05 |
| 124 | 2 | 0.4 | 0.8 | 1 | 0.05 | 0.05 |
| 125 | 0 | 0 | 0 | 3 | 0.5 | 1.5 |
| 126 | 0 | 0 | 0 | 3 | 0.9 | 2.7 |
| 127 | 0 | 0 | 0 | 3 | 0.6 | 1.8 |
| 128 | 0 | 0 | 0 | 1 | 0.4 | 0.4 |
| 129 | 0 | 0 | 0 | 2 | 0.4 | 0.8 |
| 130 | 1 | 0.2 | 0.2 | 3 | 0.9 | 2.7 |
| 131 | 2 | 0.2 | 0.4 | 3 | 0.9 | 2.7 |
| 132 | 2 | 0.1 | 0.2 | 3 | 0.8 | 2.4 |
| 133 | 0 | 0 | 0 | 3 | 0.6 | 1.8 |
| 134 | 0 | 0 | 0 | 2 | 0.7 | 1.4 |
| 135 | 0 | 0 | 0 | 2 | 0.5 | 1 |
| 136 | 0 | 0 | 0 | 2 | 0.7 | 1.4 |
| 137 | 1 | 0.1 | 0.1 | 2 | 0.4 | 0.8 |
| 138 | 1 | 0.1 | 0.1 | 3 | 0.3 | 0.9 |
| 139 | 0 | 0 | 0 | 2 | 0.8 | 1.6 |
| 140 | 0 | 0 | 0 | 2 | 0.8 | 1.6 |
| 141 | 0 | 0 | 0 | 2 | 0.7 | 1.4 |
| 142 | 0 | 0 | 0 | 2 | 0.6 | 1.2 |
| 143 | 2 | 0.1 | 0.2 | 3 | 0.6 | 1.8 |
| 144 | 3 | 0.25 | 0.75 | 1 | 0.2 | 0.2 |
| 145 | 3 | 0.3 | 0.9 | 1 | 0.3 | 0.3 |

### RL-S249/T252D Blocks Radiation-Induced PD-L1 Expression and Cancer Immunity

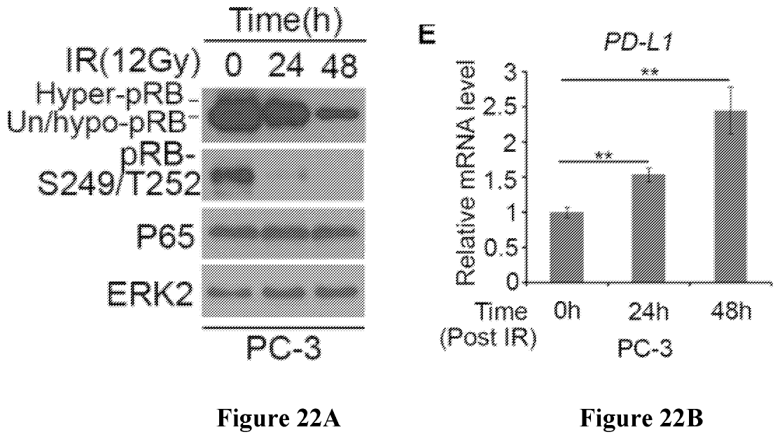

Given that radiation inhibits RB phosphorylation by inducing cell cycle arrest, it was examined if radiation increased PD-L1 expression and if this effect can be reversed by RL-S249/T252D treatment. Gamma radiation inhibited RB phosphorylation at S249/T252 and markedly increased PD-L1 expression in a time-dependent manner (FIGS. 22A and 22B), and such effect was abrogated by p65 knockdown (FIGS. 6A and 6B). Gamma radiation not only decreased S249/T252 phosphorylation and p65 binding of RB, but also increased p65 binding at the PD-L1 gene promoter (FIGS. 6C and 6D). Ectopic expression of RL-S249/T252D decreased PD-L1 basal level expression and largely diminished gamma radiation-induced upregulation of PD-L1 mRNA and protein (FIGS. 6E and 6F). These results demonstrated that gamma radiation-induced upregulation of PD-L1 was primarily mediated through the RB-NFκB signaling pathway.

The following was performed to examine whether administration of the bioactive RL-S249/T252D peptide of RB could enhance the anti-tumor efficacy of radiotherapy. PTEN-CaP8 murine prostate cancer cells infected with lentivirus of Tsin empty vector (EV) or Tsin-RL-S249D/T252D peptide were injected subcutaneously into immune-proficient mice. PTEN-CaP8 tumor-bearing mice were treated with gamma radiation (12 Gy) in combination with anti-PD-L1 antibody or non-specific control IgG (FIGS. S7F and S7G). While anti-PD-L1 antibody alone did slightly, but not dramatically inhibit tumor growth, gamma radiation alone substantially decreased tumor growth and tumor growth was completely blocked by radiation and anti-PD-L1 co-administration in the first 15 days, although tumor re-growth was observed afterwards (FIG. 6G). Consistent with the result that RL-S249/T252D peptide completely blocked Pd-l1 expression in PTEN-CaP8 cells (FIGS. 12 and 13), RL-S249/T252D treatment markedly inhibited tumor growth regardless of anti-PD-L1 treatment (FIG. 6G). In addition, tumors regressed when the small peptide was co-administrated with gamma radiation (FIG. 6G). Notably, while inhibiting Pd-l1 expression, RL-S249D/T252D peptide treatment alone also blocked expression of other NFκB targets including a few pro-survival genes (FIGS. 12 and 13), providing a plausible explanation as to how this peptide outperforms anti-PD-L1 antibody in tumor suppression. Moreover, radiation alone, but not anti-PD-L1 antibody alone, significantly increased tumor infiltration of immune effectors including CD45⁺CD8⁺ T cells and CD45⁺CD4⁺ T cells, but decreased the infiltration of CD11b⁺Gr1⁺ myeloid cells in tumors (FIG. 6H). The combination of radiation and anti-PD-L1 antibody additively increased CD45⁺CD8⁺ and CD45⁺CD4⁺ T cell infiltration in tumors (FIG. 6H). In agreement with the effect on tumor regression, co-treatment of radiation and the RL-S249/T252D peptide induced a dramatic increase in CD45⁺CD8⁺ and CD45⁺CD4⁺ T cell infiltration, but a sharp decrease of CD11b⁺Gr1⁺ myeloid cells in tumors (FIG. 6H).

### Example 3—Treating Cancer

A human identified as having cancer (e.g., PD-L1⁺ cancer such as PD-L1⁺ pancreatic cancer, PD-L1⁺ prostate cancer, PD-L1⁺ lung cancer, or PD-L1⁺ liver cancer) is administered a polypeptide that includes an amino acid sequence as set forth in any one of SEQ ID NOs: 1-179 (e.g., SEQ ID NO:2, SEQ ID NO:3, SEQ ID NO:4, or SEQ ID NO:5) at least one time a week for one to six months. After this administration is initiated, a reduction in the number of cancer cells within the human is confirmed.

### Example 4—Treating Cancer

A human identified as having cancer (e.g., PD-L1⁺ cancer such as PD-L1⁺ pancreatic cancer, PD-L1⁺ prostate cancer, PD-L1⁺ lung cancer, or PD-L1⁺ liver cancer) is administered a polypeptide that includes an amino acid sequence as set forth in any one of SEQ ID NOs:1-179 (e.g., SEQ ID NO:2, SEQ ID NO:3, SEQ ID NO:4, or SEQ ID NO:5) at least three times a week for one to six months. Radiation therapy to treat the cancer also is administered to the human. After this administration is initiated and after the radiation therapy is initiated, a reduction in the number of cancer cells within the human is confirmed.

### Example 5—Treating Cancer

A human identified as having cancer (e.g., PD-L1⁺ cancer such as PD-L1⁺ pancreatic cancer, PD-L1⁺ prostate cancer, PD-L1⁺ lung cancer, or PD-L1⁺ liver cancer) is administered a polypeptide that includes an amino acid sequence as set forth in any one of SEQ ID NOs:1-179 (e.g., SEQ ID NO:2, SEQ ID NO:3, SEQ ID NO:4, or SEQ ID NO:5) at least seven times a week for one to six months. Chemotherapy to treat the cancer (e.g., treatment with camptothecin, taxane, gemcitabine, or a combination thereof) also is administered to the human. After this administration is initiated and after the chemotherapy is initiated, a reduction in the number of cancer cells within the human is confirmed.

### Example 6—Treating Cancer

A human identified as having cancer (e.g., PD-L1⁻ cancer such as PD-L1⁻ pancreatic cancer, PD-L1⁻ prostate cancer, PD-L1⁻ lung cancer, or PD-L1⁻ liver cancer) is administered radiotherapy and/or chemotherapy, after which cancer cells within the human express an increased level of PD-L1. After this occurs, a polypeptide that includes an amino acid sequence as set forth in any one of SEQ ID NOs:1-179 (e.g., SEQ ID NO:2, SEQ ID NO:3, SEQ ID NO:4, or SEQ ID NO:5) is administered to the human at least three times a week for one to six months. After this administration is initiated and after the radiotherapy and/or chemotherapy is initiated, a reduction in the number of cancer cells within the human is confirmed.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method for treating a mammal having cancer, wherein said method comprises administering a polypeptide comprising an amino acid sequence as set forth in SEQ ID NO:1 to said mammal, wherein the level of PD-L1 expression of said cancer is reduced.

2. The method of claim 1, wherein said mammal is a human.

3. The method of claim 1, wherein said cancer is prostate cancer.

4-19. (canceled)

20. The method of claim 1, wherein said polypeptide is administered to said mammal as the sole active ingredient.

21. The method of claim 1, wherein said level of PD-L1 expression of said cancer is reduced by at least about 5 percent.

22-25. (canceled)

26. The method of claim 1, wherein the number of cancer cells present within said mammal is reduced.

27. The method of claim 1, wherein the number of cancer cells present within said mammal is reduced by at least about 10 percent.

28-29. (canceled)

30. The method of claim 1, wherein said method further comprises administering radiation to said mammal.

31. The method of claim 30, wherein said number of cancer cells within said mammal is reduced as compared to the number of cancer cells present in a comparable mammal having cancer administered said radiation and not administered said polypeptide.

32. The method of claim 30, wherein the cancer-free survival of said mammal is increased as compared to the cancer-free survival of a comparable mammal having cancer administered said radiation and not administered said polypeptide.

33. The method of claim 1, wherein said method further comprises administering a chemotherapeutic agent to said mammal.

34. The method of claim 33, wherein said chemotherapeutic agent is camptothecin, taxane, a kinase inhibitor, gemcitabine, or a combination thereof.

35. The method of claim 33, wherein said number of cancer cells within said mammal is reduced as compared to the number of cancer cells present in a comparable mammal having cancer administered said chemotherapeutic agent and not administered said polypeptide.

36. The method of claim 33, wherein the cancer-free survival of said mammal is increased as compared to the cancer-free survival of a comparable mammal having cancer administered said chemotherapeutic agent and not administered said polypeptide.

37. A polypeptide comprising an amino acid sequence as set forth in SEQ ID NO:1.

38-55. (canceled)

56. A nucleic acid molecule comprising a nucleic acid sequence encoding a polypeptide comprising an amino acid sequence as set forth in SEQ ID NO:1.

57. The nucleic acid molecule of claim 56, wherein said molecule is an expression vector.

58. The nucleic acid molecule of claim 57, wherein said expression vector is a plasmid.

59. The nucleic acid molecule of claim 56, wherein said molecule is a viral vector.

60. The nucleic acid molecule of claim 56, wherein said viral vector is a pTsin lentiviral vector.
